(19) United States
(12) Patent Application Publication
(10) Pub. No.: US 2023/0174988 A1
(43) Pub. Date: Jun. 8, 2023

(54) METHODS FOR INDUCING BILE ACID SULFOTRANSFERASE SULT2A FOR TREATING METABOLIC DISORDERS

(71) Applicants: President and Fellows of Harvard College, Cambridge, MA (US); The Brigham and Women's Hospital, Inc, Boston, MA (US)

(72) Inventors: Abigail Sloan Devlin, Cambridge, MA (US); Snehal N. Chaudhari, Cambridge, MA (US); Eric Garland Sheu, Brookline, MA (US); David A. Harris, Middleton, WI (US)

(73) Assignees: President and Fellows of Harvard College, Cambridge, MA (US); The Brigham and Women's Hospital, Inc, Boston, MA (US)

(21) Appl. No.: 17/923,891

(22) PCT Filed: May 7, 2021
(86) PCT No.: PCT/US2021/031277

§ 371 Date: Nov. 7, 2022

Related U.S. Application Data
(60) Provisional application No. 63/022,066, filed on May 8, 2020.

(51) Int. Cl. C12N 15/113; A61K 31/575; C07J 9/00

(52) U.S. Cl. CPC C12N 15/1138; A61K 31/575; C07J 9/00; C12N 2310/14

(57) ABSTRACT

The compositions and methods provided herein are related, in part, to the discovery of cholic acid 7-sulfate as a treatment for diabetes. Selective transport of the microbial metabolite lithocholic acid (LCA) from the gut to the liver after bariatric surgery activates hepatic vitamin D receptor (VDR), thereby inducing expression of bile acid sulfotransferase SULT2A, which produces the antidiabetic molecule CA7S. Provided herein is a method for treating diabetes, obesity, or an inflammatory disease in a subject, the method comprises administering to a subject in need thereof a compound of Formula I-XV or Formula F-XV′ or an agent that increases levels or activity or cholic acid 7-sulfate or upstream targets in a subject.

### RELATED APPLICATIONS

This application is a national stage filing under 35 U.S.C. § 371 of International PCT Application PCT/US2021/031277, filed May 7, 2021, which claims priority under 35 U.S.C. § 119(e) to U.S. provisional application, U.S. Ser. No. 63/022,066, filed May 8, 2020, each of which is incorporated herein by reference.

### GOVERNMENT SUPPORT

This invention was made with government support under GM128618 and DK057521 awarded by the National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Nov. 7, 2022, is named H082470362US01-SEQ-DMF and is 21,706 bytes in size.

### FIELD OF THE INVENTION

The present disclosure relates to the treatment of metabolic disorders (e.g., diabetes, obesity), or an inflammatory disease.

### BACKGROUND

Obesity and diabetes have become two of the most pressing health concerns in the United States. As a chronic condition, obesity increases the risk of developing serious diseases and disorders, including type II diabetes, cardiovascular disease, hypertension, inflammatory diseases, and some forms of cancer. Diabetes mellitus is a disease that is characterized by the lack of insulin production (e.g. type-I diabetes) by the pancreas or a lack of insulin sensitivity (e.g. type-II diabetes). Diabetes can result in a number of long term complications including diabetic ketoacidosis, hyperglycemia, or death. Serious long-term complications include cardiovascular disease, stroke, chronic kidney disease, foot ulcers, and damage to the eyes. Current treatments such as insulin injections manage the symptoms but do not prevent the long term complications of the disease and require constant monitoring of blood glucose levels. It has recently been shown that bariatric surgery results in an almost immediate resolution of diabetic symptoms. However, the mechanism remains unknown and surgery is highly invasive and costly. Thus, new treatments for diabetes, obesity, and inflammatory diseases are needed to improve the quality of life and prevent future complications of the disease.

### SUMMARY OF THE INVENTION

The compositions and methods provided herein are related, in part, to the discovery of cholic acid 7-sulfate as a treatment for metabolic disorders (e.g., diabetes, obesity) and inflammatory diseases. See, e.g., PCT publication No. WO 2020/041673, filed Aug. 23, 2019, and PCT Publication No. WO 2020/117945, filed Dec. 4, 2019. Selective transport of the microbial metabolite lithocholic acid (LCA) from the gut to the liver after bariatric surgery activates hepatic vitamin D receptor (VDR), thereby inducing expression of bile acid sulfotransferase SULT2A, which produces the antidiabetic molecule cholic acid 7-sulfate (CA7S).

In one aspect of any of the embodiments, provided herein is a method for treating metabolic disorders (e.g., diabetes, obesity), or an inflammatory disease in a subject, the method comprising administering to a subject in need thereof a compound of Formula (I):

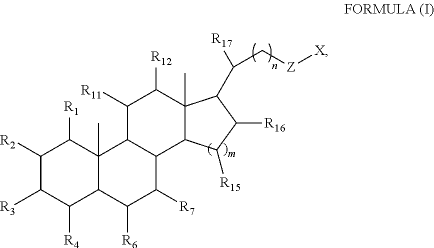

wherein:

n is 1, 2, 3, 4, 5, 6, 7, 8, 9, or 10;

m is 1, 2, 3, or 4;

Z is —C(O)—, —C(O)O—, —C(O)NR₁₈—, or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid (e.g., taurine);

each R₁, R₂, R₃, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃⁻², —OPO₃⁻², —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂,

each R₃, R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

or a pharmaceutically acceptable salt thereof.

In another aspect of any of the embodiments, provided herein is a compound of Formula (I):

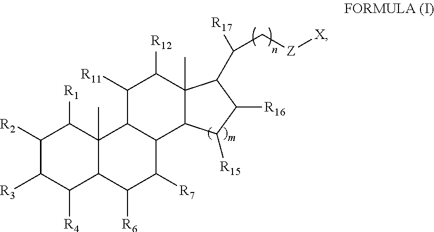

wherein:

n is 1, 2, 3, 4, 5, 6, 7, 8, 9, or 10;

m is 1, 2, 3, or 4;

Z is —C(O)—, —C(O)O—, —C(O)NR₁₈—, or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid (e.g., taurine);

each R₁, R₂, R₃, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂,

each R₃, R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

or a pharmaceutically acceptable salt thereof.

In another aspect of any of the embodiments, provided herein is a pharmaceutical composition comprising a compound of Formula (I) and a pharmaceutically acceptable carrier or excipient, wherein compound of Formula (I) has the structure:

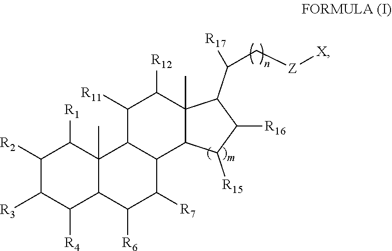

wherein:

n is 1, 2, 3, 4, 5, 6, 7, 8, 9, or 10;

m is 1, 2, 3, or 4;

Z is —C(O)—, —C(O)O—, —C(O)NR₁₈—, or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid (e.g., taurine);

each R₁, R₂, R₃, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂,

each R₃, R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

or a pharmaceutically acceptable salt thereof.

Also provided herein are compounds of Formula (I′):

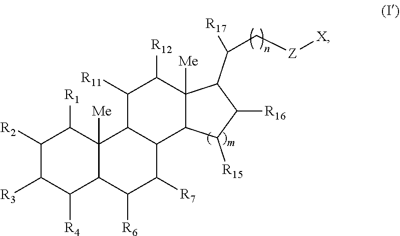

wherein:

n is 1, 2, 3, 4, 5, 6, 7, 8, 9 or 10;

m is 1, 2, 3 or 4;

Z is —C(O)—, —C(O)O—, —C(O)NR₁₈— or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid;

each R₁, R₂, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

R₃ is —OR₁₉;

each R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an oxygen protecting group, a nitrogen protecting group, or a sulfur protecting group;

R₁₉ is an oxygen protecting group;

or a pharmaceutically acceptable salt thereof.

In certain embodiments, the compounds of Formula (I′) are of the formula:

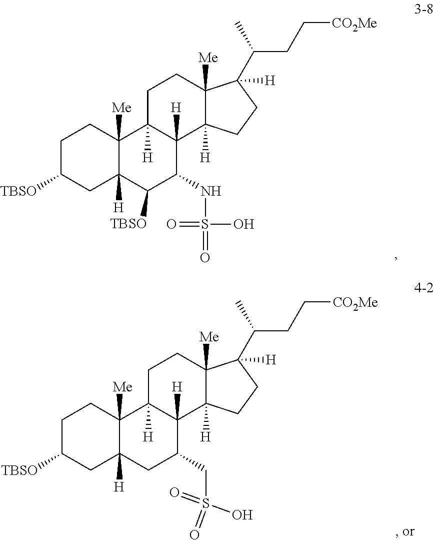

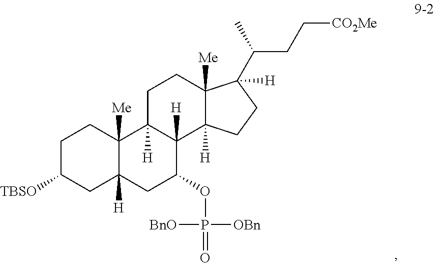

or a pharmaceutically acceptable salt thereof.

In another aspect of any of the embodiments, provided herein is a method for treating diabetes, obesity, or an inflammatory disease, the method comprises: administering to a subject in need thereof an agent that increases levels or activity of cholic acid 7-sulfate in the subject.

In another aspect of any of the embodiments, provided herein is a composition comprising an agent that increases levels or activity of cholic acid 7-sulfate in a subject.

In another aspect of any of the embodiments, provided herein is a method for treating diabetes, obesity, or an inflammatory disease, the method comprises: administering to a subject in need thereof a genetically engineered microorganism or population thereof, that expresses an agent that increases levels or activity of cholic acid 7-sulfate.

In another aspect of any of the embodiments, provided herein is a method for treating diabetes, obesity, or an inflammatory disease the method comprising: administering to a subject in need thereof a genetically engineered microorganism or population thereof, that secretes cholic acid 7-sulfate.

In another aspect of any of the embodiments, provided herein is a method for treating diabetes, obesity, or an inflammatory disease in a subject, the method comprises: administering to a subject in need thereof an agent that increases levels or activity of sulfotransferase in the subject.

In another aspect of any of the embodiments, provided herein is a method for treating diabetes, obesity, or an inflammatory disease in a subject, the method comprises: administering to a subject in need thereof an agent that increases levels or activity of lithocholic acid (LCA) in the subject.

In another aspect of any of the embodiments, provided herein is a method for treating diabetes, obesity, or an inflammatory disease in a subject, the method comprises: administering to a subject in need thereof an agent that increases levels or activity of vitamin D receptor in the subject.

In another aspect of any of the embodiments, provided herein is a method of increasing sulfotransferase levels in a cell, the method comprises: increasing levels or activity of VDR in said cell.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1A-B shows that mice are a suitable model for bariatric surgery-induced amelioration of diabetic phenotypes. FIG. 1A shows the glucose levels from sham and SG mice before surgery. FIG. 1B shows sham and SG mice glucose levels following bariatric surgery. High fat diet (HFD) mice post-sleeve show improved glucose tolerance and insulin sensitivity.

FIG. 2A-B shows that bile acid profiling reveals significant changes in individual bile acids including cholic acid 7-sulfate in mice post-sleeve. FIG. 2A shows that mice 6 weeks post-sleeve have higher levels of cholic acid 7-sulfate in their cecum compared to sham-operated mice. FIG. 2B shows that sleeve mice livers also showed increased cholic acid 7-sulfate, and reduced levels of CDCA, and TCDCA.

FIG. 3A-D shows that cholic acid 7-sulfate is a TGR5 agonist and induces GLP-1 secretion in vitro. FIG. 3A shows that sleeve mice show an increase in GLP-1 in systemic circulation. FIG. 3B shows that cholic acid 7-sulfate induces GLP-1 secretion in vitro better than the known GLP-1 inducer TDCA, while cholic acid had no effect. FIG. 3C shows that cholic acid 7-sulfate extracted from cecum of mice also has activity in inducing GLP-1 secretion in vitro. FIG. 3D shows that cholic acid 7-sulfate activates TGR5 in L-cells. The dose response curve shows an EC₅₀ of 0.013 micromolar (μM).

FIG. 4A-H shows that acute cholic acid 7-sulfate treatment induces GLP-1 and reduces serum glucose levels in vivo. FIG. 4A shows that cholic acid 7-sulfate is stable in a wide range of pHs. FIG. 4B shows that cholic acid 7-sulfate is not toxic to intestinal Caco cells in vitro. FIG. 4C-D shows that treatment of HFD-fed mice with cholic acid 7-sulfate in vivo reduced blood glucose levels and induced GLP-1 levels within 15 minute of treatment. FIG. 4E shows that dosing with 1 mg cholic acid 7-sulfate resulted in ˜2500 μM cholic acid 7-sulfate in the cecum, similar to the amounts were observed in sleeve-operated mice. FIG. 4F-G shows that ectopic introduction of cholic acid 7-sulfate allowed only minor amounts to leak into systemic circulation and into the portal vein. This did not significantly affect other bile acids in the cecum, blood, or the portal vein. FIG. 4H shows that feces from human patients pre- and post-sleeve gastrectomy also have an increase in cholic acid 7-sulfate.

FIG. 5A-H shows that portal vein bile acids induce synthesis of cholic acid 7-sulfate via SULT2A1 enzyme. FIG. 5A shows that livers from mice exhibit an increase in SULT2A enzyme isoform 1, previously shown to sulfate bile acids. FIG. 5B shows that the portal vein has a different repertoire of bile acids compared to circulating blood. FIG. 5C shows that the bile acid pool in the portal vein of sleeve-operated mice significantly induced SULT2A1 compared to the portal vein bile acid pool in sham-operated mice. FIG. 5D-E show that there was no difference in induction of SULT2A1 between the pools of bile acids mimicking those observed in the antibiotic-treated sleeve- and sham-operated mouse portal veins. FIG. 5D also shows that LCA, TDCA, CA, and CDCA were absent in the antibiotic-treated mouse portal veins. FIG. 5F shows that LCA induced SULT2A1 in HepG2, while others did not in all concentrations tested. FIG. 5G shows the relative expression of SULT2A of siRNA treated groups. FIG. 5H shows the relative expression of PXR in the liver of Sham and SG mice.

FIG. 6 shows that total bile acids and other bile acids did not differ significantly in the cecum of mice with sleeve or sham surgery.

FIG. 7 shows that total bile acids and other bile acids did not differ significantly in the liver of mice operated with sleeve or sham surgery.

FIG. 8A-C shows that cholic acid 7-sulfate-mediated induction of GLP-1 requires TGR5. FIG. 8A shows that knockdown of TGR5 abolished GLP-1 secretion. FIG. 8B shows that cholic acid 7-sulfate increases calcium levels in L-cells in vitro. FIG. 8C shows that cholic acid 7-sulfate induces TGR5 activation in HEK293T cells.

FIG. 9 shows that ectopic introduction of cholic acid 7-sulfate allowed only minor amounts to leak into systemic circulation and into the portal vein. This did not significantly affect other bile acids in the cecum, blood, or the portal vein.

FIG. 10 shows that ectopic introduction of cholic acid 7-sulfate allowed only minor amounts to leak into systemic circulation and into the portal vein. This did not significantly affect other bile acids in the cecum, blood, or the portal vein.

FIG. 11 also shows that ectopic introduction of cholic acid 7-sulfate allowed only minor amounts to leak into systemic circulation and in the portal vein. This did not significantly affect other bile acids in the cecum, blood, or the portal vein.

FIG. 12 shows that human fecal samples post-sleeve have a reduction in levels of secondary bile acids LCA, iso-LCA, and UDCA, similar to what was observed in mice post-sleeve. Other bile acids and total bile acids were not significantly affected, except for CA levels.

FIG. 13 shows that the portal vein had a very different repertoire of bile acids compared to circulating blood.

FIG. 14 shows that there is no cholic acid 7-sulfate in the liver and approximately 200-fold lower levels of cholic acid 7-sulfate in the cecum in antibiotic-treated mice compared to HFD-fed conventional mice.

FIG. 15 shows TCDCA levels of Sham and SG mice.

FIG. 16A-I shows cholic acid 7-sulfate (CA7S), a bile acid metabolite increased in mice and humans following sleeve gastrectomy and that cholic acid 7-sulfate is a TGR5 agonist that induces GLP-1 secretion in vivo. FIG. 16A shows intraperitoneal glucose tolerance test (IPGTT; AUC [95% CI], sham 51422 [46838-56006] vs SG 37251 [33735-40768]). FIG. 16B shows insulin tolerance test (ITT) performed on mice 5-weeks and 4-weeks post-surgery, respectively (SG, n=7; sham, n=6; t test, *p<0.05, **p<0.01). FIG. 16C shows GLP-1 levels were increased in mice post-SG compared to post-sham (n=11 per group, **p<0.01, Welch's t test). FIG. 16D shows the structure of CA7S. FIG. 16E shows CA7S was increased in cecal contents of SG mice (sham, n=12, SG, n=15, *p<0.05, Welch's t test). FIG. 16F shows CA7S was increased in livers of SG mice (n=12 per group, *p<0.05, Welch's t test). FIG. 16G shows CA7S in human feces was increased post-SG compared to pre-surgery (n=17 patients, *p<0.05, paired t test). FIG. 16H shows dose response curves for human TGR5 activation in HEK293T cells overexpressing human TGR5 for CA7S, TDCA, CA (≥3 biological replicates per condition). FIG. 16I shows CA7S induced secretion of GLP-1 in NCI-H716 cells compared to both CA and the known TGR5 agonist, TDCA. SiRNA-mediated knockdown of TGR5 abolished GLP-1 secretion (≥3 biological replicates per condition, one-way ANOVA followed by multiple comparisons test, *p<0.05,**p<0.01). All data are presented as mean t SEM.

FIG. 17A-E shows acute CA7S treatment induces GLP-1 and reduces serum glucose levels in vivo. FIG. 17A shows a schematic of the acute treatment experiment wherein anesthetized DIO mice were treated with PBS or CA7S via duodenal and rectal catheters.

FIG. 17B shows the concentration of CA7S in mouse cecum 15 minutes after treatment with PBS or CA7S (PBS, n=7; CA7S, n=8 mice per group, **p<0.01, Welch's t test). FIG. 17C-D shows CA7S-treated mice displayed increased GLP-1 (c, *p<0.05, Welch's t test) and reduced blood glucose levels (d, **p<0.01, Welch's t test) compared to PBS-treated mice. FIG. 17E shows the percentage cell viability upon treatment of Caco-2 cells with CA7S in vitro (≥3 biological replicates per condition, one-way ANOVA followed by multiple comparisons test; not significant). All data are presented as mean t SEM.

FIG. 18A-B shows the NMR spectroscopy and identification of cholic acid 7-sulfate (CA7S). FIG. 18A shows the structure of CA7S and the 1H NMR of authentic sample of cholic acid 7-sulfate (Cayman Chemical). FIG. 18B shows the 1H NMR of CA7S purified from the cecal contents of SG mice using UPLC-MS.

FIG. 19A-B shows UPLC-MS data. FIG. 19A shows commercially available cholic acid 7-sulfate (Cayman Chemical) and FIG. 19B shows CA7S purified from the cecal contents of SG mice have the same mass (487.2 m/z) and elute at 9.2 minutes.

FIG. 20A-E shows CA7S activates TGR5 and induces GLP-1 secretion. FIG. 20A shows CA7S induced secretion of GLP-1 in NCI-H716 cells compared to both CA and the known TGR5 agonist, TDCA. SiRNA-mediated knockdown of TGR5 abolished GLP-1 secretion (23 biological replicates per condition, one-way ANOVA followed by multiple comparisons test *p<0.05, **p<0.01, ***p<0.001). FIG. 20B shows quantitative real time PCR analysis of expression of human TGR5 in TGR5 siRNA and negative (−) siRNA-treated NCI-H716 cells for FIG. 16I and FIG. 19A. FIG. 20C shows CA7S (500 μM) purified from SG mouse cecal contents induced secretion of GLP-1 in NCI-H716 cells compared to DMSO control (**p<0.01, Welch's t test). FIG. 20D shows CA7S induced an increase in intracellular calcium levels in NCI-H716 cells (23 biological replicates per condition *p<0.05, **p<0.01, t test). FIG. 20E shows UPLC-MS traces of CA7S after incubation at 37° C. in buffer at the indicated physiological pHs.

FIG. 21 shows synthesis of 7-sulfated bile acids. Synthesis of gram quantities (minimum of about 2 grams to about 10 grams) of cholic acid 7-sulfate (CA7S).

FIG. 22 shows synthesis of milligram quantities (about 100 mg each) of CA7S variants for structure-activity studies.

FIG. 23 shows structure activity relationships (SAR) for bile acids (BA). C6 β-OH BA have lower EC50s than C6 α-OH; C7 α-OH BA have lower EC50s than C6 β-OH; these data suggest that α-muricholic acid may be the preferred core on which to test sulfation due to its C6 β-OH and C7 α-OH.

FIG. 24 shows the design and synthesis of milligram quantities synthetic/non-natural CA7S derivatives. These compounds maintain the potency of or are more potent than CA7S (i.e., lower EC50 values as TGR5 agonists), and remain gut-restricted (i.e., not absorbed into synthetic circulation).

FIG. 25 shows additional derivatives of cholic acid 7-sulfate.

FIG. 26 shows several moieties that can replace the sulfate group at position 7 (R₇) of cholic acid 7-sulfate.

FIG. 27 shows several moieties that can be added to the R₆ position of cholic acid 7-sulfate and include modifications (e.g. polar groups) that can restrict the compound to the gut.

FIG. 28A-E shows sleeve gastrectomy (SG)-mediated increase in levels of CA7S requires a microbiome. FIG. 28A shows a schematic of SG and Sham surgery in diet-induced obese (DIO) mice. FIG. 28B shows quantitative real time PCR quantification of mSult2A1 and mSult2A2 expression levels in mouse livers normalized to ribosomal protein L32 mRNA (Sham, SG n=11; *p<0.05, ns=not significant, Student's t test). FIG. 28C-D shows CA7S levels in cecum (FIG. 28C) or liver (FIG. 28D) of DIO mice that were fully colonized, treated with antibiotics (+Abx.) or in a germ-free (GF) background (Cecum: DIO, n=12, DIO+Abx., n=9, DIO; GF, n=8; Liver: DIO, n=9, DIO+Abx., n=10, DIO; GF, n=8; **p<0.01, ****p<0.0001, Welch's t test). FIG. 28E shows quantitative real time PCR quantification of mSult2A1 expression level in livers of DIO, DIO+Abx, or in DIO; GF mice normalized to ribosomal protein L32 mRNA (DIO, n=9, DIO+Abx., n=10, DIO; GF, n=8; **p<0.01, Student's t test).

FIG. 29A-D shows portal vein bile acids induce expression of SULT2A1 in hepatocytes in vitro. FIG. 29A shows portal vein bile acids in DIO mice post-Sham and SG (Sham, n=5; SG n=4; not significant, Welch's test). FIG. 29B shows Quantitative real time PCR quantification of human SULT2A1 expression level in HepG2 cells treated with indicated concentration of DIO portal vein bile acids normalized to human GAPDH (□3 biological replicates per condition *p<0.05, **p<0.01, Student's test). FIG. 29C shows portal vein bile acids in DIO mice treated with antibiotics post-Sham and SG (Sham, n=5; SG n=5; not significant, Welch's test). FIG. 29D shows quantitative real time PCR quantification of human SULT2A1 expression level in HepG2 cells treated with indicated concentration of DIO+Abx. portal vein bile acids normalized to human GAPDH (≥3 biological replicates per condition, *p<0.05, **p<0.01, Student's test).

FIG. 30A-F shows LCA induces expression of SULT via the Vitamin D receptor (VDR). FIG. 30A shows quantitative real time PCR quantification of human SULT2A1 expression level in HepG2 cells treated with indicated concentration of bile acids normalized to human GAPDH (≥3 biological replicates per condition, p value shown only for induction of SULT2A1, *p<0.05, **p<0.01, Student's test). FIG. 30B shows synthesis of CA7S in HepG2 cells upon treatment with indicated bile acids and cofactor PAPS (≥3 biological replicates per condition, p value shown only for production of CA7S, *p<0.05, Student's test). FIG. 30C shows siRNA-mediated knockdown of VDR significantly abolishes LCA-mediated induction of SULT2A1 in HepG2 cells compared to negative control siRNA (≥3 biological replicates per condition, *p<0.05, Student's test). FIG. 30D shows quantitative real time PCR quantification of VDR expression levels in mouse livers normalized to ribosomal protein L32 mRNA (Sham, SG n=11; *p<0.05, Student's t test). FIG. 30E shows quantitative real time PCR quantification of VDR expression level in livers of DIO, DIO+Abx, or in DIO; GF mice normalized to ribosomal protein L32 mRNA (DIO, n=9, DIO+Abx., n=10, DIO; GF, n=8; **p<0.01, Student's t test). FIG. 30F shows model—(1) SG induces a (2) shift in the microbiome which (3) induces transport of bacterially-derived secondary bile acid LCA in the portal vein. (4) LCA induces expression of the bile acid-SULT in the liver via VDR which leads to production of CA7S. (5) CA7S induces GLP-1 secretion in the intestine via TGR5, which can improve systemic hyperglycemia.

FIG. 31A-E show again that LCA induces expression of SULT via the Vitamin D receptor (VDR). FIG. 31A shows CA7S levels in feces of wild-type and VDR knockout mice (WT, n=4, VDRKO, n=7, *p=0.01, Welch's t test). FIG. 31B shows a schematic of portal vein injection with LCA. FIG. 31C-D shows quantitative real time PCR quantification of Sult2A1 (FIG. 31C) and VDR (FIG. 31D) expression levels in mouse livers injected with LCA or PBS normalized to ribosomal protein L32 mRNA (n=3; for (G) *p=0.02, for (H) *p=0.03, Welch's t test). FIG. 31E shows CA7S levels in the gall bladder of mice injected with LCA or PBS normalized to ribosomal protein L32 mRNA (n=3; *p=0.02, Welch's t test). All data are presented as mean t SEM.

FIG. 32A-C shows microbiome-modified secondary bile acids are lower in mice and humans post-SG. FIG. 32A shows production of primary bile acids in the liver and secondary bile acids in the intestine. FIG. 32B shows LCA, isoLCA, and total bile acid levels in Sham and SG mouse cecums (Sham, n=12; SG n=15; *p<0.05, ns=not significant, Welch's test). FIG. 32C shows LCA, isoLCA, UDCA, and total bile acid levels in human feces pre- and post-SG (n=17, *p<0.05, ns=not significant, Welch's t test).

FIG. 33A-I shows 16S rRNA sequencing analysis of mouse cecal contents and human feces reveal a shift in the microbiome post-SG. FIG. 33A shows a schematic of mice subjected to cecal 16S sequencing (Sham, n=15; SG, n=17). FIG. 33B-C shows principal component analysis (PCoA) (FIG. 33B), taxa summary (FIG. 33C) of sham- and SG-mouse microbiome. FIG. 33D shows relative abundance of Clostridiales in sham and SG mice (Sham, n=15; SG, n=17, p=0.16, ns=not significant, Welch's t test). FIG. 33E shows quantitative real time PCR quantification of baiCD gene cluster expression levels in sham and SG mouse cecal contents normalized to bacterial 16S ribosomal DNA (Sham, n=10, SG, n=14, *p=0.02, Welch's t test). FIG. 33F shows schematic of the human patient samples subjected to fecal 16S sequencing (n=17). FIG. 33G-H shows principal component analysis (PCoA) (FIG. 33G), taxa summary (FIG. 33H) in pre- and post-human feces. FIG. 33I shows relative abundance of Clostridiales in pre- and post-human feces (n=17, **p=4.80×10⁻³, paired t test). All data are presented as mean t SEM.

FIG. 34A-F shows intestinal BA transport proteins Asbt and Ostα facilitate selective transport of LCA into the portal vein. FIG. 34A shows a schematic of intestinal BA transport. FIG. 34B shows quantitative real time PCR quantification of bile acid (BA) transport protein expression levels in sham and SG mouse distal ileum normalized to ribosomal protein L32 mRNA (Sham, n=15; SG, n=17; Asbt **p=8.80×10-3, Ostα *p=0.04, Ostβ p=0.89, iBabp p=0.76, Bsep p=0.64, Mrp1 p=0.89, Mrp2 p=0.89, Mrp3 p=0.35, Oatp1 p=0.78, Oatp2 p=0.68, Oatp4 p=0.84, ns=not significant, Welch's t test). FIG. 34C shows SEM images of undifferentiated and differentiated Caco-2 cells in transwells. Scale bar from left to right equals 400 μm, 20 μm, and 4 μm). FIG. 34D shows a schematic of BA transport study. FIG. 34E shows quantification of indicated BAs transported across differentiated Caco-2 cells that are either untreated or treated with Asbt, Ostα, Asbt+Ostα siRNA, or U0216 (≥3 biological replicates per condition). FIG. 34F shows corresponding AUC measurements for (E) (≥3 biological replicates per condition, *p<0.05, ^{#}p<0.01 ^{†}p<1.00×10⁻³, data not marked are not significant; CA (Asbt) p=0.99, CA (Ostα) p=0.91, CA (Asbt+Ostα) p=0.97, CA (U0126) p=0.65; CDCA (Asbt) p=0.78, CDCA (Ostα) p=0.73, CDCA (Asbt+Ostα) p=0.73, CDCA (U0126) p=0.38; βMCA (Asbt) p=0.81, βMCA (Ostα) p=0.74, βMCA (Asbt+Ostα) p=0.81, βMCA (U0126) p=0.99; TβMCA (Asbt) p=0.99, TβMCA (Ostα) p=0.66, TβMCA (Asbt+Ostα) p=0.83, TβMCA (U0126) p=0.31; TCA (Asbt) *p=0.01, TCA (Ostα) ^{†}p=7.00×10⁻⁴, TCA (Asbt+Ostα) ^{†}p=1.70×10⁻⁴, TCA (U0126) ^{†}p=1.00×10⁻⁴; DCA (Asbt) ^{†}p=6.00×10⁻⁴, DCA (Ostα) *p=0.02, DCA (Asbt+Ostα) *p=0.04, DCA (U0126) p=0.08; LCA (Asbt) *p=0.03, LCA (Ostα) *p=0.02, LCA (Asbt+Ostα) *p=0.01, LCA (U0126) ^{#}p=2.80×10⁻³, one-way ANOVA followed by Turkey's (HSD) post-hoc test). All data are presented as mean t SEM.

FIG. 35A-F shows LCA induces CA7S synthesis and GLP-1 secretion in vitro. FIG. 35A shows a schematic of SG and Sham surgery in diet-induced obese (DIO) mice treated with or without antibiotics (Abx.). FIG. 35B-C shows GLP-1 levels in systemic circulation of mice subjected to SG or Sham surgeries treated with or without antibiotics (data not marked with asterisk(s) are not significant FIG. 35B, Sham n=3, SG n=6, *p=0.01; FIG. 35C, n=5 in each group, p=0.26, Welch's t test). FIG. 35D shows a schematic of co-culture study. FIG. 35E shows synthesis of CA7S in HepG2 cells upon incubation with indicated treatments (≥3 biological replicates per condition, data marked with asterisk(s) are only for production of CA7S; CA+LCA+PAPS vs. CA+PAPS **p=2.70×10⁻³, CA+LCA+PAPS vs. CA+LCA+PAPS+VDR siRNA **p=1.60×10⁻³, one-way ANOVA followed by Dunnett's multiple comparisons test). FIG. 35E shows synthesis of CA7S in HepG2 cells upon incubation with indicated treatments (≥3 biological replicates per condition, data marked with asterisk(s) are only for production of CA7S; CA+LCA+PAPS vs. CA+PAPS **p=2.70×10⁻³, CA+LCA+PAPS vs. CA+LCA+PAPS+VDR siRNA **p=1.60×10⁻³, one-way ANOVA followed by Dunnett's multiple comparisons test). FIG. 35F shows GLP-1 secretion assay in NCI-H716 cells co-cultured with HepG2 cells (≥3 biological replicates per condition, data not marked by asterisk(s) are not significant, DMSO vs. CA p=0.96; DMSO vs. CA+PAPS p=0.26; DMSO vs. CA+LCA p=0.12; DMSO vs. CA+LCA+PAPS ****p<1.00×10⁻⁴; DMSO vs. CA+LCA+PAPS+VDR siRNA p=0.49; CA vs. CA+LCA+PAPS ***p=3.00×10⁻³; CA+LCA vs. CA+LCA+PAPS *p=0.04; CA+PAPS vs. CA+LCA+PAPS *p=0.01; CA+LCA+PAPS vs. CA+LCA+PAPS+VDR siRNA *p=0.02, one-way ANOVA followed by Dunnett's multiple comparisons test). All data are presented as mean t SEM.

FIG. 36A and FIG. 36B shows CA7S levels in cecal contents of mice subjected to SG or sham surgeries treated with or without antibiotics (Abx.) (A) Sham n=7, SG n=6, *p=0.01; (B) n=5 in each group, ns=not significant p=0.26, Welch's t test. FIG. 36C and FIG. 36D shows GLP-1 levels in systemic circulation of mice subjected to SG or sham surgeries treated with or without antibiotics. For FIG. 36C, sham n=3, SG n=6, *p=0.01; for FIG. 36D, n=5 in each group, p=0.26, Welch's t test).

FIG. 37 shows the expression of hSULT2A, as measured by qRT-PCR, was increased in conventional sham PV BA-treated cells and decreased in antibiotic sham PV BA-treated cells relative to DMSO control. hSULT2A expression was normalized to human GAPDH (R3 biological replicates per condition, data not marked with asterisk(s) are not significant, 100 mM DMSO versus Sham **p=5.60 3 10_3; 100 mM DMSO versus sham-Abx. p=0.82; 100-mM sham versus Sham-Abx. ***p=1.00 3 10_4; 500 mM DMSO versus Shamp=0.29; 500 mMDMSO versus Sham-Abx. p=0.98; 500-mMsham versus Sham-Abx. *p=0.04; 1,000 mMDMSO versus Sham*p=0.03; 1,000 mMDMSO versus Sham-Abx. p=0.97; 1,000-mM sham versus Sham-Abx. **p=2.30 3 10_3, two-way ANOVA followed by Dunnett's multiple comparisons test). All data are presented as mean t SEM.

FIG. 38A shows the mammalian sulfotransferase enzyme SULT2A (mSULT2A in mice and hSULT2A in humans) catalyzes the conversion of the primary bile acid CA to cholic acid-7-sulfate (CA7S). FIG. 38B shows the qRT-PCR quantification of mSult2A isoforms reported to sulfate bile acids in the murine liver. Expression levels were normalized to 18S. n=11 in each group, mSult2A1 *p=0.04, mSult2A2 p=0.92, mSult2A8 p=0.78, Welch's t test). FIG. 38C shows the hepatic expression of mSult2A1, as measured by qRT-PCR, was significantly higher in DIO mice than in DIO+Abx. mice and DIO; GF mice. Expression levels were normalized to mouse ribosomal 18S (DIO, n=9, DIO+Abx., n=9, DIO; GF, n=8; DIO versus DIO+Abx. ****p<1 3 104, DIO versus DIO; GF ****p<1 3 104, one-way ANOVA followed by Dunnett's multiple comparisons test). All data are presented as mean t SEM.

FIG. 39A-H shows LCA is sufficient to inhibit Asbt expression and induce production of CA7S. FIG. 39A shows a schematic of sham and SG intestine BA transport modulated by the production of LCA by Clostridia. Levels of Clostridia and LCA production are higher in sham mice. LCA inhibits Asbt expression, resulting in less transport of LCA into the portal vein. In contrast, levels of Clostridia and LCA are lower in SG mice, allowing for higher expression of Asbt and increased transport of LCA from the gut to the liver in SG animals. FIG. 39B shows the sham cecal pool of BAs inhibited expression of ASBT compared with the SG cecal pool of BAs (3 biological replicates per condition, sham versus SG cecal pool *p=0.02, one-way ANOVA followed by Dunnett's multiple comparisons test. FIG. 39C shows LCA (100 mM) inhibited expression of ASBT in Caco-2 cells. (3 biological replicates per condition, *p=0.02, Welch's t test). FIG. 39D shows a schematic of GF mice administered 0.3% LCA (w/w) in chow for 1 week prior to harvesting tissues for analyses. FIG. 39E shows LCA feeding led to accumulation of LCA in the proximal small intestine (SI), distal ileum (DI), and cecum of GF mice. (n=5 in each group, SI *p=0.04; DI *p=0.04; cecum **p=1.70 3 10_3, Welch's t test). FIG. 39F shows LCA inhibited expression of Asbt in SI and DI (n=5 in each group, SI p=0.11; DI *p=0.02, Welch's t test). (G) Introduction of LCA in GF mice induced CA7S production and accumulation in the gallbladder (GB) and the DI (GB, GF n=3, GF+LCA n=4, **p=6.50310_3; DI, n=5 in each group, *p=0.03, Welch's t test). FIG. 39H shows LCA feeding led to increased expression of mSult2A1 and VDR in livers of mice fed 0.3% LCA in chow (n=5 in each group, mSult2A1 *p=0.04, VDR p=0.09, Welch's t test). All data are presented as mean t SEM.

FIG. 40A-D shows compounds, e.g., cholic acid-7-phosphate (CA7P, Compound 9), Compound 9-2, Compound 4-2, and Compound 3-8, induces TGR5 expression. FIG. 40B-D show the relative TGR5 expression of the various compounds.

FIG. 41A-F shows CA7S has anti-inflammatory effects in vitro and in vivo. FIG. 41A shows CA7S induces secretion of the anti-inflammatory cytokine IL-10 from macrophages in vitro. FIG. 41B shows NK-FB activation increases inflammation. FIG. 41C shows an in vitro assay using CA7S in vitro with THP1-Blue cells (a human macrophage cell line), with an NF-kB reporter. FIG. 41D shows CA7S reduces NFkB activation in THP1 cells and protects against Lipopolysaccharides (LPS)-induced inflammation. FIG. 41E shows CA7S does not affect macrophage cell viability. FIG. 41F shows CA7S improves inflammation in vivo.

### DETAILED DESCRIPTION OF THE INVENTION

Obesity and type 2 diabetes (T2D or type-II diabetes) are medical pandemics. Bariatric surgery, in the form of Roux-en-Y gastric bypass or sleeve gastrectomy (SG), is currently the most effective and durable treatment for obesity and related comorbidities^{1,2}. Owing to robust post-surgical metabolic benefits and favorable side-effect profile, SG is the most common bariatric surgery performed in the US ³. While maximal weight loss occurs at 1 year, many patients see resolution of their T2D within days of surgery⁴. For a majority of patients, remission is durable, lasting for at least 7 years^{1,4}. The molecular mechanisms underlying T2D remission, however, remain largely unknown⁵.

Two consistently observed post-surgical changes are increased levels of GLP-1, a circulating incretin hormone, and changes in the systemic repertoire of bile acids (BAs). BAs are cholesterol-derived metabolites that play crucial roles in host metabolism by acting as detergents that aid in the absorption of lipids and vitamins and as ligands for host receptors⁶. The therapeutic benefits of GLP-1 and the causal role of bile acids in mediating beneficial metabolic changes post-surgery are provided herein.

The compositions and methods provided herein are related, in part, to the discovery that cholic acid 7-sulfate is increased in subjects following bariatric surgery and ameliorates the symptoms of diabetes. Also provided herein is evidence that cholic acid 7-sulfate is a TGR5 agonist and induces GLP-1 secretion in vitro.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

### Diabetes, Obesity, and Inflammatory Diseases

In another aspect of any of the embodiments, provided herein is a method of treating diabetes in a subject. As used herein the term “diabetes mellitus” or “diabetes” refers to any disease that affects the release of insulin from the pancreas (e.g. type I diabetes) or the sensitivity to insulin (e.g. type II diabetes). Diabetes can cause at least one symptom of the disease or patients can be asymptomatic. The symptoms associated with diabetes include but are not limited to, malaise, blurred vision, hunger, frequent urination, increased thirst, or any other symptom associated with the disease in a subject.

In one embodiment of any of the aspects, the diabetes is type I diabetes, type II diabetes, neonatal diabetes, maturity onset diabetes in the young, or gestational diabetes. The cause of diabetes can be due to a genetic mutation, inherited diabetes, obesity, lifestyle, or idiopathic.

Generally, diabetes is characterized and diagnosed by high blood glucose levels in a subject's serum (e.g. hyperglycemia). The diagnosis can be carried out by a physician with a glucose challenge test and/or a glucose tolerance test. For an oral glucose tolerance test in humans, a blood sugar level less than about 140 mg/dL (7.8 mmol/L) is normal. A reading of more than about 200 mg/dL (11.1 mmol/L) after two hours indicates that the subject has diabetes.

Diabetes can cause many complications and symptoms. Symptoms of diabetes include but are not limited to increased thirst, frequent urination, increased hunger, unintended weight loss, fatigue, blurred vision, slow healing sores, frequent infections, and areas of darkened skin. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), inflammatory diseases, chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation.

The methods and compositions provided herein can further be applied to treat or prevent prediabetes in a subject. Prediabetes is a condition in which blood glucoses levels are elevated but they are not severe enough for a diagnosis of type II diabetes. A blood glucose reading between about 140 and about 199 mg/dL (7.8 mmol/L and 11.0 mmol/L) can indicate prediabetes. The symptoms of pre-diabetes are similar to diabetes and include but are not limited to increased thirst, frequent urination, fatigue, and blurred vision. A subject can also be one who is suffering from or at risk of developing diabetes or a pre-diabetic condition.

In another embodiment of any of the aspects, the diabetes caused by obesity. In another aspect of any of the embodiments, provided herein is a method of treating obesity in a subject. The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Some common conditions related to high BMI and obesity include cardiovascular disease, high blood pressure (i.e., hypertension), osteoarthritis, cancer, and diabetes. Although BMI correlates with body fat, the relation between BMI and actual body fat differs with age and gender. For example, women are more likely to have a higher percent of body fat than men for the same BMI. Furthermore, the BMI threshold that separates normal, overweight, and obese can vary, e.g. with age, gender, ethnicity, fitness, and body type, amongst other factors.

In some embodiments of any of the aspects, a subject with obesity can be a subject with a body mass index of at least about 25 kg/m² prior to administration of a treatment as described herein. In some embodiments, a subject with obesity can be a subject with a body mass index of at least about 30 kg/m² prior to administration of a treatment, compound, or agent as described herein.

In another aspect of any of the embodiments, provided herein is a method of treating an inflammatory disease in a subject. As used herein, the term “inflammation” or “inflamed” or “inflammatory” refers to activation or recruitment of the immune system or immune cells (e.g. T cells, B cells, macrophages). A tissue that has inflammation can become reddened, white, swollen, hot, painful, exhibit a loss of function, or have a film or mucus. Methods of identifying inflammation are well known in the art. Inflammation generally occurs following injury or infection by a microorganism.

As used herein the term “an inflammatory disease” refers to any disease that affects the immune system. The inflammatory disease can cause at least one symptom of the disease. These symptoms can include but are not limited to, diarrhea, vomiting, nausea, upset stomach, pain, swollen joints, malaise, fever, weight loss, weight gain, bleeding, any change in the consistency or frequency of a bowel movement or stool, or any other symptom associated with an inflammatory disease in a subject. In some embodiments, the inflammatory disease is an autoimmune disease.

In one embodiment of any of the aspects, the inflammatory disease is selected from the group consisting of: Crohn's disease, inflammatory bowel disease, ulcerative colitis, pancreatitis, hepatitis, appendicitis, gastritis, diverticulitis, celiac disease, food intolerance, enteritis, ulcer, and gastroesophageal reflux disease (GERD), psoriatic arthritis, psoriasis, and rheumatoid arthritis, or any other inflammatory disease known in the art.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

In one embodiment of any of the aspects, the subject is a mammal. In another embodiment of any of the aspects, the subject is a human. As provided herein, the mammal can be a human, non-human primate, mouse, rat, dog, cat, horse, or cow, but is not limited to these examples. Mammals other than humans can be advantageously used as subjects that represent animal models of disease e.g., diabetic or obesity model. A subject can be male or female.

A subject can be one who has been previously diagnosed with or identified as suffering from or having a disease or disorder in need of treatment (e.g., diabetes, obesity, or an inflammatory disease) or one or more complications related to such a disease or disorder, and optionally, have already undergone treatment for the disease or disorder or the one or more complications related to the disease or disorder. Alternatively, a subject can also be one who has not been previously diagnosed as having such disease or disorder or related complications. For example, a subject can be one who exhibits one or more risk factors for the disease or disorder or one or more complications related to the disease or disorder or a subject who does not exhibit risk factors.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with diabetes, obesity, or an inflammatory disease. The term “treating” includes reducing or alleviating at least one adverse effect or symptom of diabetes. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

### Compounds and Chemical Modifications

The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). An alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. An alkylene is au uncyclized chain. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. A heteroalkyl is an uncyclized chain. The heteroatom(s) O, N, P, S, B, As, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃—, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent. means. unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy. alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. A heteroalkylene is an uncyclized chain. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms. mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. A cycloalkyl or heteroalkyl is not aromatic. Examples of cycloalkyl include, but are not limited to, cyclopropyl, cyclobutyl, cyclopentyl, cyclohexyl, 1-cyclohexenyl, 3-cyclohexenyl. cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean. unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl. trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together. wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl. purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6.5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system. heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, the oxygen protecting group is TBDPS, TBS, TIPS, TES, or TMS. In certain embodiments, the oxygen protecting group is TBS.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl. heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R′R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R′, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR″R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group. for example, each of the R groups is independently selected as are each R′, R″, R′″, and R″″ groups when more than one of these groups is present.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, at least one oxygen protecting group is silyl, TBDPS, TBDMS, TIPS, TES, TMS, MOM, THP, t-Bu, Bn, allyl, acetyl, pivaloyl, or benzoyl.

Substituents for rings (e.g. cycloalkyl, heterocycloalkyl, aryl, heteroaryl, cycloalkylene. heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring. fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen. while obeying the rules of chemical valency.

Two or more substituents may optionally be joined to form aryl, heteroaryl, cycloalkyl, or heterocycloalkyl groups. Such so-called ring-forming substituents are typically. though not necessarily, found attached to a cyclic base structure. In one embodiment of any of the aspects. the ring-forming substituents are attached to adjacent members of the base structure. For example, two ring-forming substituents attached to adjacent members of a cyclic base structure create a fused ring structure. In another embodiment of any of the aspects. the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R′, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:

(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:
(i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, and heteroaryl, substituted with at least one substituent selected from:
   (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl. each substituted or unsubstituted heteroalkyl is a substituted or unsubstituted 2 to 20 membered heteroalkyl, each substituted or unsubstituted cycloalkyl is a substituted or unsubstituted C₃-C₈cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry. as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

The term “silyl ether” as used herein, refers to a chemical compound containing a silicon atom covalently bonded to an alkoxy group generally having the structure R^{w}R^{x}R^{y}Si—O—R^{z}, wherein R^{w}, R^{x}, R^{y}, and R^{z} are independently alkyl or aryl groups.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium. potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric. monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of salts include mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. The term salt also refers to formation of a salt between two compounds.

Certain compounds of the present invention possess asymmetric carbon atoms (optical or chiral centers) or double bonds: the enantiomers. racemates. diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diasteromeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

In one aspect of any of the embodiments, provided herein is a method for treating diabetes, the method comprising administering to a subject in need thereof a compound of Formula (I):

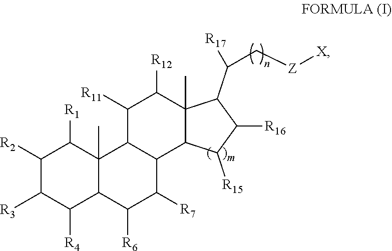

wherein:

n is 1, 2, 3, 4, 5, 6, 7, 8, 9, or 10;

m is 1, 2, 3, or 4;

Z is —C(O)—, —C(O)O—, —C(O)NR₁₈—, or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid (e.g., taurine);

each R₁, R₂, R₃, R₄, R₁₁, R₁₂, R₁₈, R₁₆, and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂,

each R₃, R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇, and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

or a pharmaceutically acceptable salt thereof.

In another aspect of any of the embodiments, provided herein is a compound of Formula (I). In still another aspect, provided herein is a pharmaceutical composition comprising a compound of Formula (I) and a pharmaceutically acceptable carrier or excipient.

In another aspect of any of the embodiments, provided herein is a compound of Formula (I′). In still another aspect, provided herein is a pharmaceutical composition comprising a compound of Formula (I′) and a pharmaceutically acceptable carrier or excipient.

In some embodiments of the various aspects disclosed herein, the compound of Formula (I) can be a compound of any one of Formula (II)-(XV).

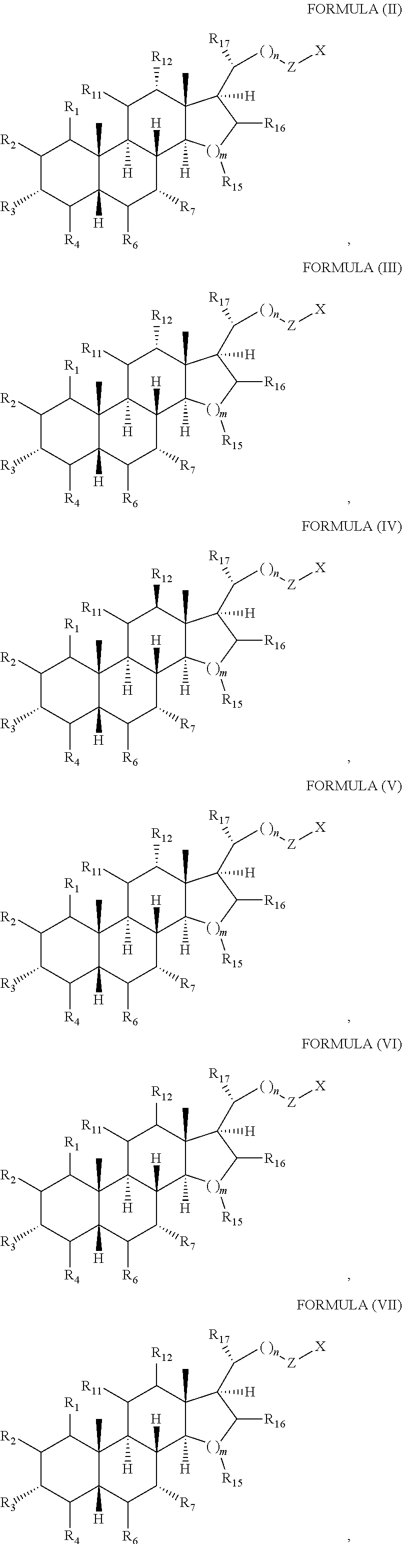

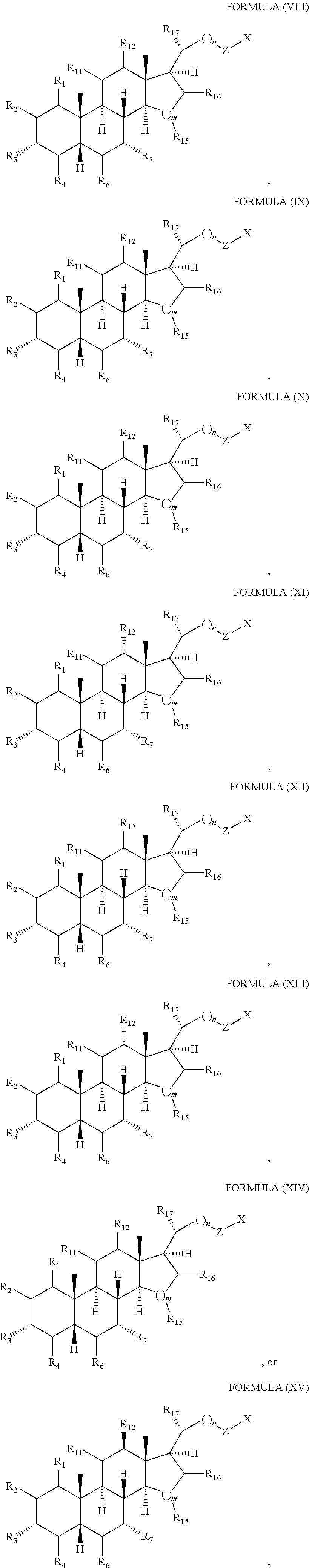

In some embodiments of the various aspects disclosed herein, R₁, R₂, R₄, R₁₅, and R₁₆ are H.

In some embodiments of the various aspects disclosed herein, R₁, R₂, R₄, R₆, R₁₁, R₁₅, and R₁₆ are H.

In some embodiments of the various aspects disclosed herein, R₁, R₂, R₄, R₁₁, R₁₅, and R₁₆ are H.

In some embodiments of the various aspects disclosed herein, R₁, R₂, R₄, R₆, R₇, R₁₁, R₁₅, and R₁₆ are H.

In some embodiments of the various aspects disclosed herein, R₃ and/or R₁₂ are OH.

In some embodiments of the various aspects disclosed herein, R₇ and/or R₁₂ are OH.

In some embodiments of the various aspects disclosed herein, R₃ and/or R₇ are OH.

In some embodiments of the various aspects disclosed herein, R₃ and/or R₆ are OH.

In some embodiments of the various aspects disclosed herein, R₆ and/or R₇ are OH.

In some embodiments of the various aspects disclosed herein, R₃ and/or R₇ are OH.

In some embodiments of the various aspects disclosed herein, R₆ and R₇ are H.

In some embodiments of the various aspects disclosed herein, R₃ is H or OH.

In some embodiments of the various aspects disclosed herein, R₁₇ is C₁-C₆ alkyl. For example, R₁₇ can be methyl, ethyl, propyl, isopropyl, butyl, pentyl, etc.

In some embodiments of the various aspects disclosed herein, n is 2.

In some embodiments of the various aspects disclosed herein, is 1.

In some embodiments of the various aspects disclosed herein, wherein at least one of R₃, R₆, R₇ and R₁₂ is —OSO₃⁻, —NR₁₈SO₃⁻, or —OPO₃²⁻.

In some embodiments of the various aspects disclosed herein, at least one of R₆, R₇ and R₁₂ is —OSO₃⁻, —NR₁₈SO₃⁻, -or —OPO₃²⁻.

In some embodiments of the various aspects disclosed herein, R₆ or R₇ is —OSO₃⁻, —NR₁₈SO₃⁻, or —OPO₃²⁻.

In some embodiments of the various aspects disclosed herein, R₆ or R₇ is —OSO₃⁻.

In some embodiments of the various aspects disclosed herein, R₇ and R₁₂ are independently —OSO₃⁻.

In some embodiments of the various aspects disclosed herein, R₃, R₆, R₇ and R₁₂ are independently H, OH, —OSO₃⁻, —NR₁₈SO₃⁻, -or —OPO₃²⁻, provided that at least one of R₃, R₆, R₇ and R₁₂ is —OSO₃⁻, —NR₁₈SO₃⁻, -or —OPO₃²⁻.

In some embodiments of the various aspects disclosed herein, R₃, R₆, R₇ and R₁₂ are independently H, OH, —OSO₃⁻, —NR₁₈SO₃⁻, -or —OPO₃²⁻, provided that at least one of R₆, R₇ and R₁₂ is —OSO₃⁻, —NR₁₈SO₃₋, -or —OPO₃²⁻.

In some embodiments of the various aspects disclosed herein, R₃, R₆, R₇ and R₁₂ are independently H, OH, —OSO₃⁻, —NR₁₈SO₃⁻, -or —OPO₃²⁻, provided that R₆ or R₇ is —OSO₃⁻, —NR₁₈SO₃⁻, -or —OPO₃²⁻.

In some embodiments of the various aspects disclosed herein, R₃, R₆, R₇ and R₁₂ are independently H, OH, —OSO₃⁻, —NR₁₈SO₃⁻, or —OPO₃²⁻, provided that at least one of R₆ or R₇ is —OSO₃⁻.

In some embodiments of the various aspects disclosed herein, R₃, and R₆ are independently H, OH, —OSO₃⁻, —NR₁₈SO₃⁻, -or —OPO₃²⁻; and R₇ and R₁₂ are independently —OSO₃⁻.

In some embodiments of the various aspects described herein, the compound of Formula (I) is of Formula (XVI):

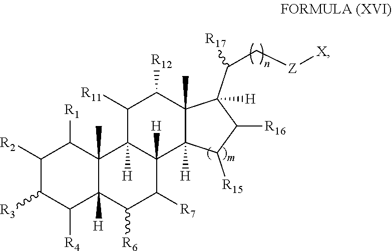

wherein X is OH or a polar amino acid (e.g., taurine); R₇ is —OSO₃H, —SO₃H, OSO₂R₁₈, —NHSO₃H, OSO₂N(R₁₈)₂, —NHSO₂R₁₈, —SO₂N(R₁₈)₂, —OPO₃H, or —ONO₂; R₁, R₂, R₄, R₆, R₁₁, R₁₅, R₁₆, R₁₈, n and m are as defined for Formula (I); R₃ is H or OH; and R₁₇ is H or methyl.

In some embodiments of the various aspects described herein, the compound of Formula (I) is of Formula (XVII):

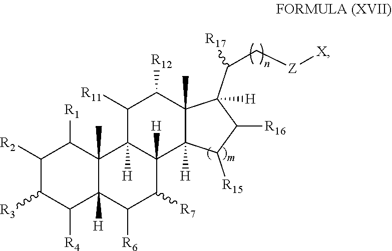

wherein X is OH or a polar amino acid (e.g., taurine); R₆ is —OSO₃H, —SO₃H, OSO₂R₁₈, —NHSO₃H, OSO₂N(R₁₈)₂, —NHSO₂R₁₈, —SO₂N(R₁₈)₂, —OPO₃H, or —ONO₂; R₁, R₂, R₄, R₇, R₁₁, R₁₅, R₁₆, R₁₈, n and m are as defined for Formula (I); R₃ is H or OH; and R₁₇ is H or methyl.

In some embodiments, of the various aspects disclosed herein, the compound of Formula (I) is not a naturally occurring bile acid. For example, the compound of Formula (I) is not cholic acid 7-sulfate.

In some embodiments, of the various aspects disclosed herein, the compound of Formula (I) is not lithocholic acid 3-sulfate.

Compounds of Formula (I) can be synthesized as shown in Scheme I.

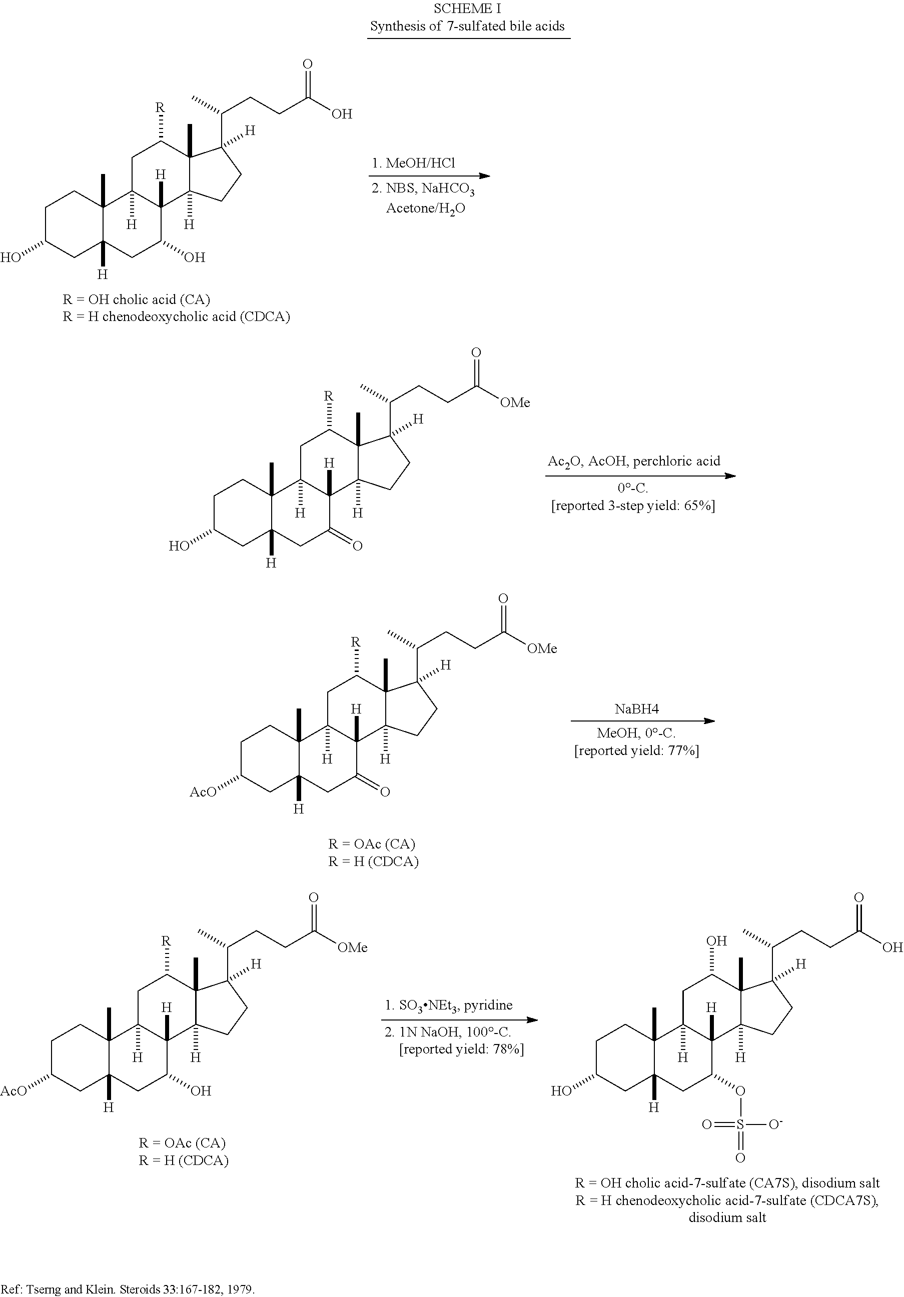

In another aspect, provided herein is a compound of Formula (I′):

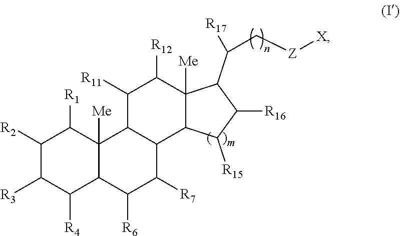

wherein:

n is 1, 2, 3, 4, 5, 6, 7, 8, 9 or 10;

m is 1, 2, 3 or 4;

Z is —C(O)—, —C(O)O—, —C(O)NR₁₈— or —CH₂—;

X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid;

each R₁, R₂, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃⁻², —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;

R₃ is —OR₁₉;

each R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;

each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, an oxygen protecting group, a nitrogen protecting group, or a sulfur protecting group;

R₁₉ is an oxygen protecting group;

or a pharmaceutically acceptable salt thereof.

In certain embodiments, the compound of Formula (I′) is of Formula (II′):

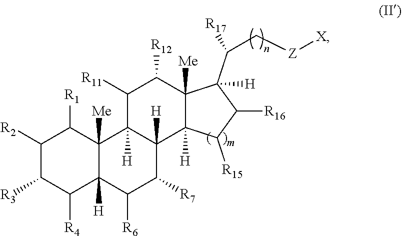

or a pharmaceutically acceptable salt thereof, wherein R₁-R₁₇, X, Z, m, and n are defined as in Formula (I′).

In certain embodiments, the compound of Formula (I′) is of Formula (III′):

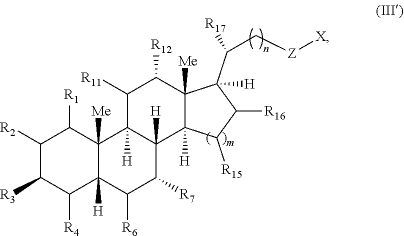

or a pharmaceutically acceptable salt thereof, wherein R₁-R₁₇, X, Z, m, and n are defined as in Formula (I′).

In certain embodiments, the compound of Formula (I′) is of Formula (IV′):

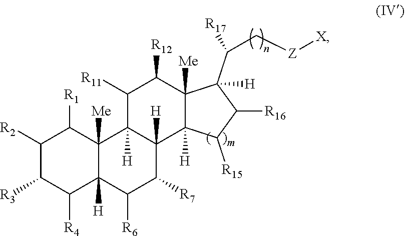

or a pharmaceutically acceptable salt thereof, wherein R₁-R₁₇, X, Z, m, and n are defined as in Formula (I′).

In certain embodiments, the compound of Formula (I′) is of Formula (V′):

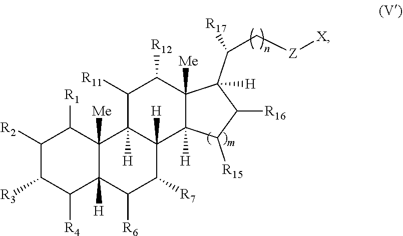

or a pharmaceutically acceptable salt thereof, wherein R₁-R₁₇, X, Z, m, and n are defined as in Formula (I′).

In certain embodiments, the compound of Formula (I′) is of Formula (VI′):

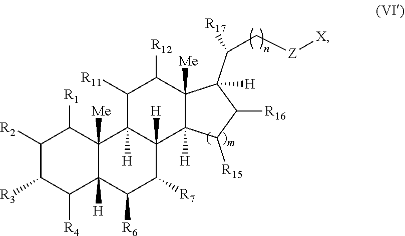

or a pharmaceutically acceptable salt thereof wherein R₁-R₁₇, X, Z, m, and n are defined as in Formula (I′).

In certain embodiments, the compound of Formula (I′) is of Formula (VII′):

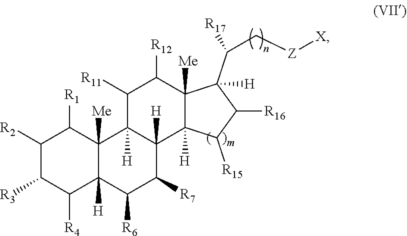

or a pharmaceutically acceptable salt thereof wherein R₁-R₁₇, X, Z, m, and n are defined as in Formula (I′).

In certain embodiments, the compound of Formula (I′) is of Formula (VIII′):

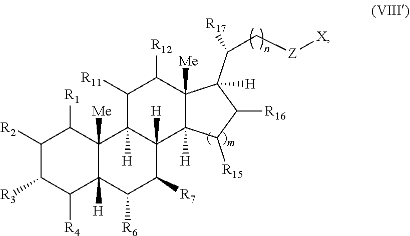

or a pharmaceutically acceptable salt thereof wherein R₁-R₁₇, X, Z, m, and n are defined as in Formula (I′).

In certain embodiments, the compound of Formula (I′) is of Formula (IX′):

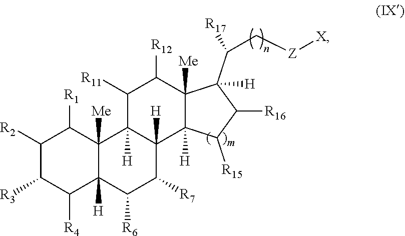

or a pharmaceutically acceptable salt thereof wherein R₁-R₁₇, X, Z, m, and n are defined as in Formula (I′).

In certain embodiments, the compound of Formula (I′) is of Formula (X′):

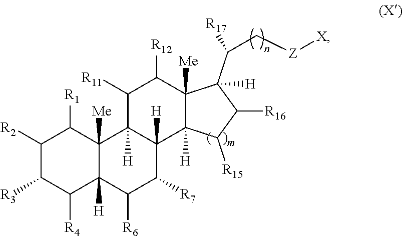

or a pharmaceutically acceptable salt thereof wherein R₁-R₁₇, X, Z, m, and n are defined as in Formula (I′).

In certain embodiments, the compound of Formula (I′) is of Formula (XI′):

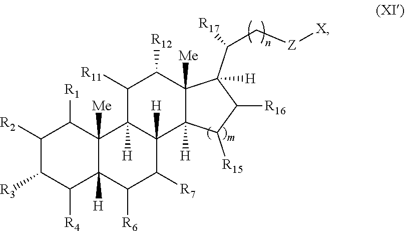

or a pharmaceutically acceptable salt thereof wherein R₁-R₁₇, X, Z, m, and n are defined as in Formula (I′).

In certain embodiments, the compound of Formula (I′) is of Formula (XII′):

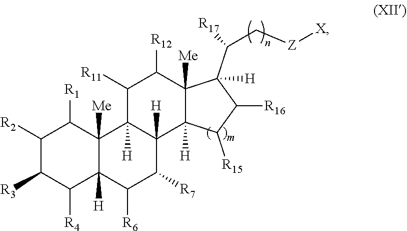

or a pharmaceutically acceptable salt thereof wherein R₁-R₁₇, X, Z, m, and n are defined as in Formula (I′).

In certain embodiments, the compound of Formula (I′) is of Formula (XIII′):

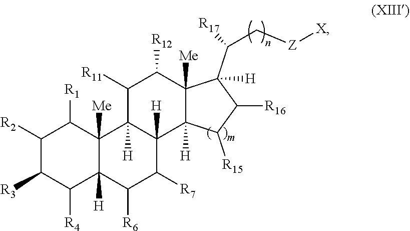

or a pharmaceutically acceptable salt thereof wherein R₁-R₁₇, X, Z, m, and n are defined as in Formula (I′).

In certain embodiments, the compound of Formula (I′) of Formula (XIV′):

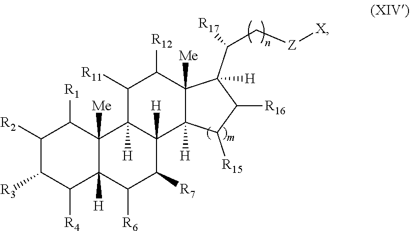

or a pharmaceutically acceptable salt thereof wherein R₁-R₁₇, X, Z, m, and n are defined as in Formula (I′).

In certain embodiments, the compound of Formula (I′) of Formula (XV′):

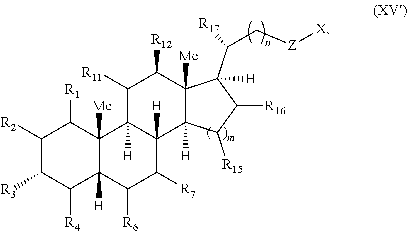

or a pharmaceutically acceptable salt thereof wherein R₁-R₁₇, X, Z, m, and n are defined as in Formula (I′).

In certain embodiments, each R₁₈ is independently hydrogen. In certain embodiments, each R₁₈ is independently benzyl. In certain embodiments, each R₁₈ is independently unsubstituted benzyl. In certain embodiments, each R₁₈ is independently an oxygen protecting group. In certain embodiments, each R₁₈ is independently a sulfur protecting group. In certain embodiments, each R₁₈ is independently a nitrogen protecting group. In certain embodiments, each R₁₈ is independently substituted or unsubstituted alkyl. In certain embodiments, each R₁₈ is independently substituted or unsubstituted heteroalkyl. In certain embodiments, each R₁₈ is independently substituted or unsubstituted cycloalkyl. In certain embodiments, each R₁₈ is independently substituted or unsubstituted heterocycloalkyl. In certain embodiments, each R₁₈ is independently substituted or unsubstituted aryl. In certain embodiments, each R₁₈ is independently substituted or unsubstituted heteroaryl.

In certain embodiments, R₇ is —OR₁₈, wherein R₁₈ is an oxygen protecting group. In certain embodiments, the oxygen protecting group is TBDPS, TBS, TIPS, TES, or TMS. In certain embodiments, the oxygen protecting group is TBS. In certain embodiments, R₁₈ is —Si(R^{aa})₃. In certain embodiments, R₁₈ is —SiMe₂t-Bu.

oxygen protecting group, a nitrogen protecting group, or a sulfur protecting group

In certain embodiments, the compound of Formula (I′) has the substituent R₃. In certain embodiments, R₃ is —OR₁₉, wherein R₁₉ is an oxygen protecting group. In certain embodiments, the oxygen protecting group is TBDPS, TBS, TIPS, TES, or TMS. In certain embodiments, the oxygen protecting group is TBS. In certain embodiments, R₁₉ is —Si(R^{aa})₃. In certain embodiments, R₁₉ is —SiMe₂t-Bu.

In certain embodiments, the compound of Formula (I′) is of the formula:

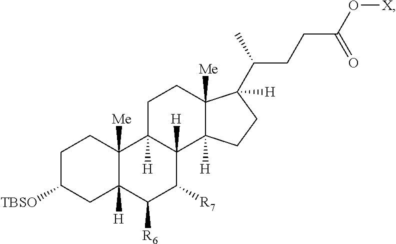

or a pharmaceutically acceptable salt thereof, wherein X, R₆, and R₇ are defined as in Formula (I′).

In certain embodiments, the compound of Formula (I′) is of the formula:

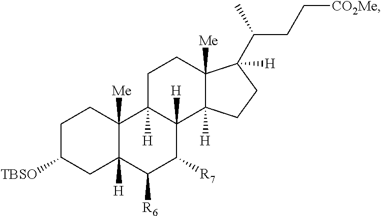

or a pharmaceutically acceptable salt thereof.

In certain embodiments, the compound of Formula (I′) is of the formula:

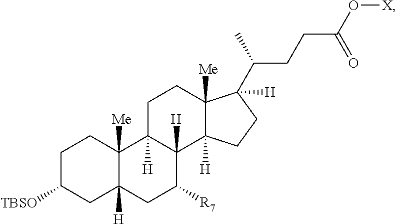

or a pharmaceutically acceptable salt thereof, wherein R₇ and X are defined as in Formula (I′).

In certain embodiments, the compound of Formula (I′) is of the formula:

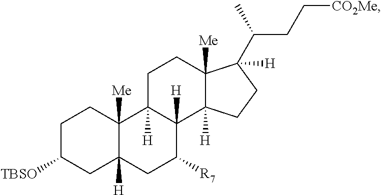

or a pharmaceutically acceptable salt thereof, wherein R₇ is defined as in Formula (I′).

In certain embodiments, the compound of Formula (I′) of the formula:

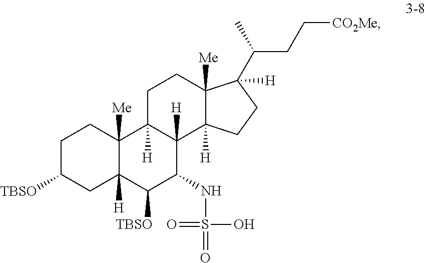

or a pharmaceutically acceptable salt thereof.

In certain embodiments, the compound of Formula (I′) of the formula:

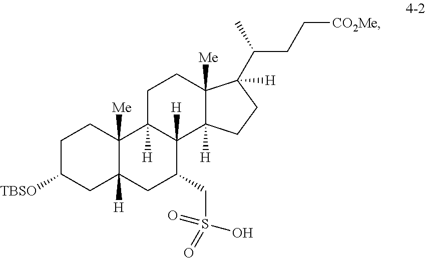

or a pharmaceutically acceptable salt thereof.

In certain embodiments, the compound of Formula (I′) of the formula:

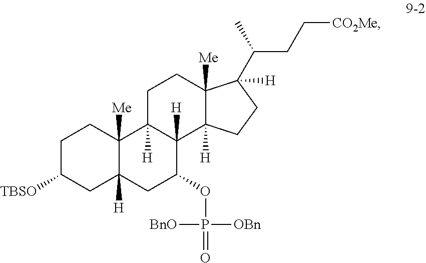

or a pharmaceutically acceptable salt thereof.

### Agents

In one aspect of any of the embodiments, provided herein is method for treating diabetes, obesity, or an inflammatory disease in a subject, the method comprises: administering to a subject in need thereof an agent that increases levels or activity of cholic acid 7-sulfate in the subject.

In another aspect of any of the embodiments, provided herein is a method for treating diabetes, obesity, or an inflammatory disease in a subject, the method comprises: administering to a subject in need thereof an agent that increases levels or activity of sulfotransferase in the subject.

In another aspect of any of the embodiments, provided herein is a method for treating diabetes, obesity, or an inflammatory disease in a subject, the method comprises: administering to a subject in need thereof an agent that increases levels or activity of lithocholic acid (LCA) in the subject.

In another aspect of any of the embodiments, provided herein is a method for treating diabetes, obesity, or an inflammatory disease in a subject, the method comprises: administering to a subject in need thereof an agent that increases levels or activity of vitamin D receptor in the subject.

In another aspect of any of the embodiments, provided herein is a method for treating diabetes, obesity, or an inflammatory disease in a subject, the method comprises administering to a subject in need thereof a compound of Formula (I)-(XVII), or derivative thereof.

In another aspect of any of the embodiments, provided herein is a method for treating diabetes, obesity, or an inflammatory disease in a subject, the method comprises administering to a subject in need thereof a compound of Formula (I′)-(XVII′), or derivative thereof.

An “agent” as used herein is a chemical molecule of synthetic or biological origin. In the context of the present invention, an agent is generally a molecule that can be used in a pharmaceutical composition.

In one embodiment of any of the aspects, the agent is selected from the group consisting of a small molecule, an antibody, a peptide, a genome editing system, an antisense oligonucleotide, shRNA, and an siRNA.

As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments, natural-product-like small molecules are utilized.

As used herein, a “compound” refers to any chemical, test chemical, drug, new chemical entity (NCE) or other moiety. For example, a compound can be any foreign chemical not normally present in a subject such as mammals including humans. A compound can also be an endogenous chemical that is normally present and synthesized in biological systems, such as mammals including humans. For example, a compound, such as a test compound, such as a drug, can induce the secretion of GLP-1 in a subject by activation of TGR5 as provided herein.

The term “derivative” as used herein means any chemical, conservative substitution, or structural modification of an agent. The derivative can improve characteristics of the agent or small molecule such as pharmacodynamics, pharmacokinetics, absorption, distribution, delivery, targeting to a specific receptor, or efficacy. For example, for a small molecule, the derivative can consist essentially of at least one chemical modification to about ten modifications. The derivative can also be the corresponding salt of the agent. The derivative can be the pro-drug of the small molecule as provided herein.

The term “RNAi” or “siRNA” or “shRNA” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA.

In one embodiment of any of the aspects, the agent that increases TGR5, VDR, and/or sulfotransferase is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides as described herein are complementary nucleic acid sequences designed to hybridize under cellular conditions to a gene. Thus, oligonucleotides are chosen that are sufficiently complementary to the target, i.e., that hybridize sufficiently well and with sufficient specificity in the context of the cellular environment, to give the desired effect. For example, an antisense oligonucleotide that activates or increases levels of TGR5, VDR, and/or sulfotransferase directly or indirectly may comprise at least 5, at least 10, at least 15, at least 20, at least 25, at least 30, or more bases complementary to a portion of the coding sequence of the human TGR5, VDR, and/or SULT2A1 gene (e.g., SEQ ID NOs: 1-7), respectively. Furthermore, the antisense oligonucleotide can target transcription factors that regulate the expression of TGR5, VDR, and/or SULT2A1 such as farnesoid X receptor, retinoid X receptor (RXR), RORγt, X-box binding protein-1 (XBP1), or any other transcription factors known in the art.

In another embodiment of any of the aspects, increasing levels or activity of TGR5, sulfotransferase, or VDR comprises administering a nucleic acid encoding TGR5, SULT2A1, or VDR to the cell.

As used herein, the term “antibody” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)₂, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include broadly neutralizing antibodies, midibodies, nanobodies, humanized antibodies, chimeric antibodies, and the like. Furthermore, the antibody as provided herein can comprise an amino acid sequence complementary to TGR5 (SEQ ID NO: 2), GLP-1 (SEQ ID NO: 3), VDR (SEQ ID NO: 4), or SULT2A1 (SEQ ID NO: 6) or binds to an amino acid sequence that comprises a sequence with at least 80%, at least 85%, at least 90%, at least 95%, at least 96%, at least 97%, at least 98%, at least 99% or greater sequence identity to the sequences of SEQ ID NOs: 2-4, 6). The antibody can directly or indirectly affect TGR5, GLP-1, VDR, or sulfotransferase levels, e.g. by binding to a transcriptional repressor protein of TGR5, GLP-1, VDR and/or SULT2A1 gene expression thereby increasing gene expression of TGR5, GLP-1, VDR, and/or SULT2A1.

In one embodiment of any of the aspects, the agent is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment of any of the aspects, the humanized antibody is a humanized monoclonal antibody. In one embodiment of any of the aspects, the humanized antibody is a humanized polyclonal antibody. In one embodiment of any of the aspects, the humanized antibody is for therapeutic use.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

In another embodiment of any of the aspects, TGR5, VDR, and/or sulfotransferase, are increased in the cell's genome using any genome editing system including, but not limited to, zinc finger nucleases, TALENS, meganucleases, and CRISPR/Cas systems. In one embodiment of any of the aspects, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The gene editing system can directly or indirectly modulate levels or activity of TGR5, VDR, and/or sulfotransferase expression, e.g. by inhibiting transcriptional repressors of these molecules that results in an increase in their transcription.

### Bile Acids and Cholic Acid 7-Sulfate

In one embodiment of any of the aspects, the agent is cholic acid 7-sulfate. In another embodiment of any of the aspects, the agent is a derivative of cholic acid 7-sulfate as provided herein. In another embodiment of any of the aspects, the agent is a bile acid or derivative thereof. In another embodiment of any of the aspects, the agent is lithocholic acid (LCA) or a derivative of LCA. In another embodiment of any of the aspects, the agent is a Vitamin-D receptor (VDR) agonist.

As used herein, the term “bile acid” refers to a steroid acid that aids digestion as emulsifiers of fat, and may also play a role in various systemic endocrine hormone-like functions. Bile acids in mammals are synthesized from cholesterol in the liver as primary bile acids and are metabolized by particular mammalian gut microbes to secondary bile acids. Bile acids in mammals regulate metabolic pathways by activation of farnesoid X receptor as well as the G-protein-coupled receptor (GPCRs) such as TGR5. Non-limiting examples of bile acids include cholic acid, glycocholic acid, taurocholic acid, deoxycholic acid, chenodeoxycholic acid, glycochenodeoxycholic acid, taurochenodeoxycholic acid (TCDA), lithocholic acid (LCA), ursodeoxycholic acid (UDCA), muricholic acids, obeticholic acid, and any other bile acid known in the art. The term “bile acid” can further refer to salt forms of bile acids, sulfated bile acids, and other metabolites.

As used herein, the term “cholic acid 7-sulfate,” or “CA7S” or “7-sulfocholic acid” refers to the sulfated form of cholic acid. The structure of cholic acid 7-sulfate is as follows:

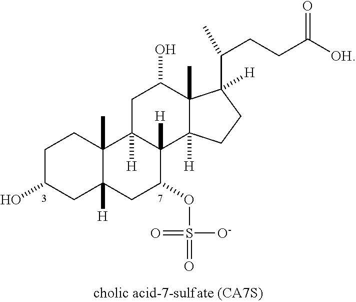

As used herein, the term “lithocholic acid,” or “LCA” or “3α-hydroxy-5β-cholan-24-oic acid” refers to another bile acid that acts as a detergent to solubilize fats for absorption and improve glucose metabolism as provided herein. The structure of LCA is as follows:

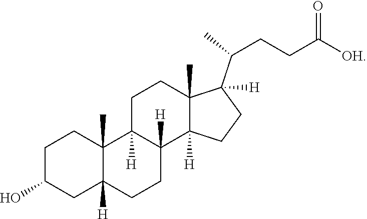

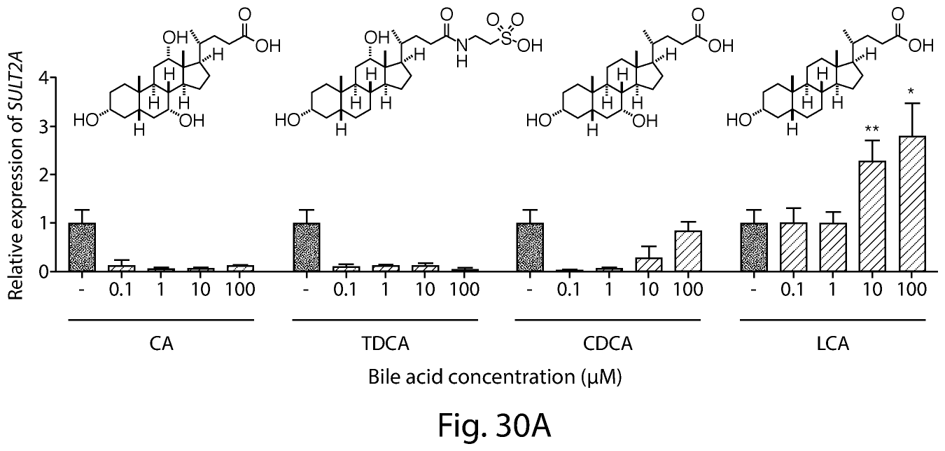

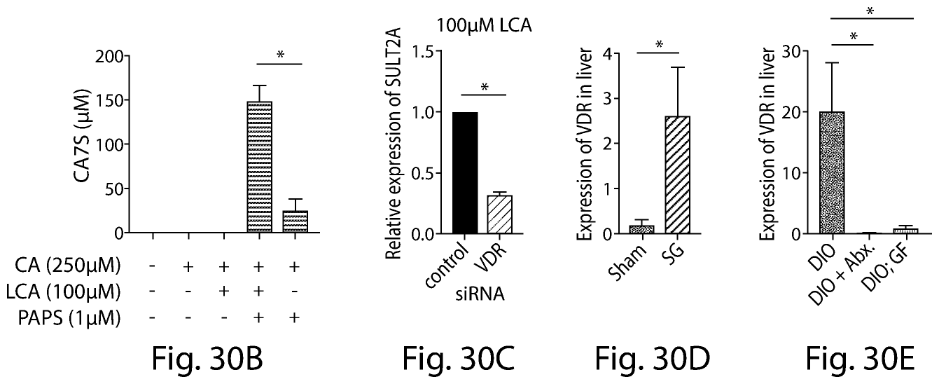

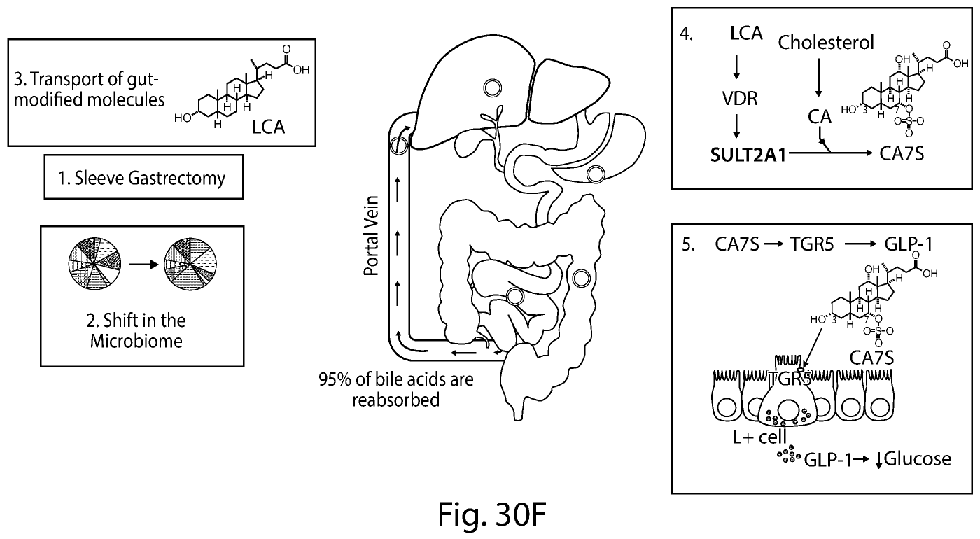

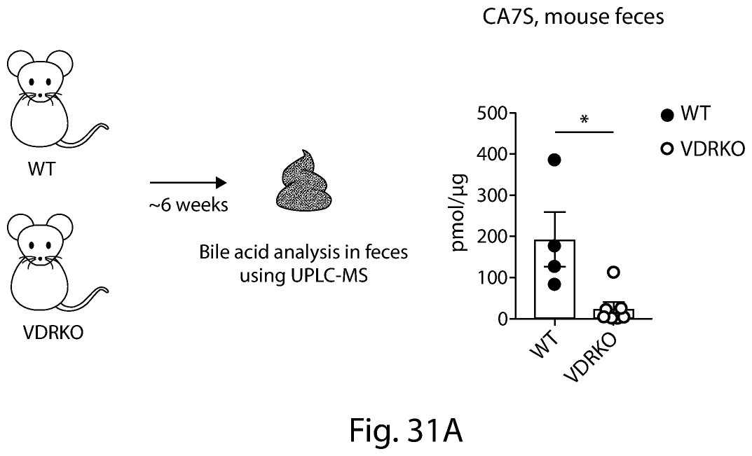

LCA can be further metabolized by intestinal bacteria into LCA isomers, including isoLCA. Without wishing to be bound by a theory, LCA can also be hydroxylated into ursodeoxycholic acid (UDCA). Provided herein LCA is shown to induce SULT2A, e.g., SULT2A1 expression in a dose dependent manner (See FIG. 30A).

### Molecular Targets for the Treatment of Diabetes, Obesity, and Inflammatory Diseases

In one embodiment of any of the aspects, the agent provided herein is a TGR5 agonist.

As used herein, the term “TGR5” or “G protein-coupled bile acid receptor 1” or GPBAR1” or “G-protein coupled receptor 19” or “GPCR19” or “membrane-type receptor for bile acids” or “M-BAR” refers to a receptor for bile acids encoded by the GPBAR1 gene (NCBI Gene ID: 2842). Sequences for TGR5 are known in the art, e.g., the human mRNA transcript (e.g. NM_006143.2, SEQ ID NO: 1), and polypeptide sequence (e.g. NP_006134.1, SEQ ID NO: 2). Bile acids activate mitogen-activated protein kinase pathways, and are ligands for the G-protein-coupled receptor (GPCR) TGR5. Activation of TGR5 then activates nuclear hormone receptors such as farnesoid X receptor a (FXR-a). Through activation of these diverse signaling pathways, bile acids can regulate their own enterohepatic circulation, but also triglyceride, cholesterol, energy, and glucose homeostasis.

As used herein, the terms “TGR 5 activity” or “activity of TGR5” refers to the cellular functions of the TGR5 receptor, for example, activation of TGR5 results in the secretion of GLP-1 from a cell (e.g. L-cells in the gut). As provided herein, an increase in TGR5 levels and activity results in an increase in GLP-1. TGR5 activity can further refer to the sensing of bile acids, metabolites, and regulation of glucose homeostasis. The activation of TGR5 or an increase in TGR5 activity as provided herein can also refer to an increase in the production of intracellular cAMP, activation of MAP kinase signaling pathways, internalization of the receptor, suppression of macrophage function or immune functions, and regulation of bile acid synthesis, degradation, or function. While the activation of TGR5 in macrophages decreases pro-inflammatory cytokine production, the stimulation of TGR5 by bile acids in adipocytes and myocytes enhances energy expenditure. TGR5 activity can increase as a result of activation by cholic acid 7-sulfate, CA7S derivatives, or any ligand or agonist of TGR5.

Non-limiting examples of TGR5 agonists include triazole, imidazole, cholesterol and derivatives of cholesterol, RUP43, 6-methyl-2-oxo-4-thiophen-2-yl-1,2, 3, 4,-tetrahydropyrimidine-5-carboxylic acid benzyl ester, 3-Aryl-4-isoxazolecarboxamides or any other TGR5 agonists known in the art.

In another embodiment of any of the aspects, the TGR5 agonist induces GLP-1 secretion from a target cell. In some embodiments of any of the aspects, the target cell is an enteroendocrine cell, an epithelial cell, an L-cell, or a neuron.

As used herein, the term “glucagon-like peptide-1” or “GLP-1” refers to a peptide hormone that is 30 amino acids long that is derived from the pro-glucagon peptide. GLP-1 is produced primarily by enteroendocrine cells in the gut (e.g. L-cells). However, other cell types such as neurons can produce GLP-1. GLP-1 has the ability to decrease blood glucose levels in a glucose-dependent manner by enhancing insulin secretion from the pancreas. GLP-1 has also been shown in enhance the insulin gene transcription, replenish insulin stores in the pancreas, and promote pancreatic beta cell growth. GLP-1 further inhibits gastric emptying, acid secretion, motility, and decreases appetite. The polypeptide sequence of GLP-1 can be found in SEQ ID NO: 3.

As used herein, the term “Vitamin D-Receptor,” or “VDR,” “Vitamin D3-receptor” or “calcitriol receptor” or “NR1I1” refers to a receptor for vitamin D that is expressed in nearly every major organ in the body to regulate the expression of specific gene products and transcriptional responses and functions as a receptor for bile acids. Sequences for VDR, are known for a number of species, e.g., human vitamin D receptor (NCBI Gene ID: 7421 and NCBI Reference Sequence NG_008731.1) polypeptide and mRNA (e.g., NCBI Reference Sequences: NP_001017535.1, NP_001017536.1 and NM_000376.2, NM_000376.2). VDR can refer to human VDR, including naturally occurring variants, molecules, genetically engineered VDR, and alleles thereof. Vitamin D receptor refers to the mammalian vitamin D receptor of, e.g., mouse, rat, rabbit, dog, cat, cow, horse, pig, and the like. The amino acid sequence of the VDR is shown in SEQ ID NO: 4. The mRNA transcript sequence is shown in SEQ ID NO: 5.

As used herein, the term “sulfotransferase” or “SULT2A” encoding dehydroepiandrosterone sulfotransferase (DHEAST) or “sulfotransferase 2A” is used to described the gene, transcript, or protein enzyme that catalyzes the sulfate conjugation of hormone, neurotransmitters, drugs, and other compounds. Further, as used herein, “SULT2A” encompasses all isoforms of SULT2A including, but not limited to, SULT2A1.

SULT2A1, specifically, is expressed in the liver and adrenal glands, among others. As provided herein, sulfation of bile acids tags them for excretion from the body. Sequences for SULT2A1, are known for a number of species, e.g., human SULT2A1 (NCBI Gene ID: 6822 and NCBI Reference Sequence: NG_016745.1) polypeptide sulfotransferase 2A1 and mRNA (e.g., NCBI Reference Sequences: NP_003158.2 and NM_003167.4). SULT2A1 can refer to human SULT2A1, including naturally occurring variants, molecules, genetically engineered SULT2A1, and alleles thereof. SULT2A1 refers to the mammalian SULT2A1 receptor of, e.g., mouse, rat, rabbit, dog, cat, cow, horse, pig, and the like. The amino acid sequence of the sulfotransferase 2A1 is shown in SEQ ID NO: 6. The mRNA transcript sequence for SULT2A1 is shown in SEQ ID NO: 7. Sulfotransferase can refer to any sulfotransferase variant or gene family member currently known or yet to be discovered.

In one aspect of any of the embodiments, provided herein is a method of increasing sulfotransferase levels in a cell, the method comprises: increasing levels or activity of VDR in said cell.

As used herein, the terms “VDR activity” or “activity of VDR” refers to the cellular functions of the vitamin D receptor, for example, activation of VDR results in induction of SULT2A1 in a cell (e.g. hepatocytes). As provided herein, an increase in VDR levels and activity results in an increase in SULT2A1 and subsequently cholic acid 7-sulfate, TGR5 activation, and/or GLP-1 secretion from L-cells. VDR activity can increase as a result of LCA signaling, or any derivative of LCA, or any ligand or agonist of VDR.

As used herein, the terms “sulfotransferase activity,” or “activity of sulfotransferase,” or “SULT2A activity” or “activity of SULT2A” refers to the cellular functions of the sulfotransferase. For example, activation of SULT2A, e.g., SULT2A1 results in the sulfation of bile acids in a cell (e.g. hepatocytes). As provided herein, an increase in VDR levels and activity results in an increase in SULT2A, e.g., SULT2A1 and subsequently cholic acid 7-sulfate and GLP-1 secretion from L-cells. SULT2A, e.g., SULT2A1 activity can increase as a result of contact with bile acids and their derivatives (e.g. LCA), xenobiotics, aliphatic hydroxyl groups, hydroxysteroids, or any activator of the sulfotransferase enzymes.

In one embodiment of any of the aspects, the increasing levels or activity of VDR comprises administering an agonist of VDR.

In another embodiment of any of the aspects, the increasing levels or activity of VDR comprises administering LCA or derivative of LCA to the cell.

In another embodiment of any of the aspects, increasing levels or activity of VDR comprises administering a nucleic acid encoding VDR to the cell. In another embodiment of any of the aspects, the nucleic acid encoding VDR is SEQ ID NO: 5 or NCBI Reference Sequence NG_008731.1.

In another embodiment of any of the aspects, the increasing levels or activity of TGR5 comprises administering a nucleic acid encoding TGR5 to the cell. In another embodiment of any of the aspects, the nucleic acid encoding TGR5 is SEQ ID NO: 1.

In another embodiment of any of the aspects, the increasing levels or activity of sulfotransferase comprises administering a nucleic acid encoding sulfotransferase to the cell. In another embodiment of any of the aspects, the sulfotransferase is SULT2A1. In another embodiment of any of the aspects, the nucleic acid encoding SULT2A1 is SEQ ID NO: 7.

In another embodiment of any of the aspects, the increasing levels or activity of VDR, sulfotransferase, and/or TGR5 are in vivo. In another embodiment of any of the aspects, the increasing levels or activity of VDR, sulfotransferase, and/or TGR5 are in a mammal. In another embodiment of any of the aspects, the increasing levels or activity of VDR, sulfotransferase, and/or TGR5 are in a human. In another embodiment of any of the aspects, the increasing levels or activity of VDR, sulfotransferase, and/or TGR5 are in a subject in need of treatment for diabetes, obesity, or an inflammatory disease.

In another embodiment of any of the aspects, the activity of TGR5 is increased by at least 50%, at least 60%, at least 70%, at least 80%, at least 90%, or more as compared to an appropriate control.

In another embodiment of any of the aspects, the secretion of GLP1 is increased by at least 50%, at least 60%, at least 70%, at least 80%, at least 90%, or more as compared to an appropriate control.

In another embodiment of any of the aspects, the activity of VDR is increased by at least 50%, at least 60%, at least 70%, at least 80%, at least 90%, or more as compared to an appropriate control.

In another embodiment of any of the aspects, the activity of sulfotransferase is increased by at least 50%, at least 60%, at least 70%, at least 80%, at least 90%, or more as compared to an appropriate control.

As used herein, an “appropriate control” refers to an untreated, otherwise identical cell or population (e.g., a subject who was not administered an agent provided herein, or was administered by only a subset of agents provided herein, as compared to a non-control cell).

### Pharmaceutical Compositions

In one embodiment of any of the aspects, the agent or compound as provided herein is formulated with a pharmaceutical composition.

In one aspect of any of the embodiments, provided herein is a composition comprising an agent that increases levels or activity of cholic acid 7-sulfate in a subject. In one embodiment of any of the aspects, the agent is cholic acid 7-sulfate. In another embodiment of any of the aspects, the agent is a derivative of cholic acid 7-sulfate. In another embodiment of any of the aspects, the composition is formulated for treating diabetes, obesity, or an inflammatory disease. In another embodiment of any of the aspects, the composition further comprises a pharmaceutically acceptable carrier or excipient.

As used herein, the term “pharmaceutical composition” can include any material or substance that, when combined with an active ingredient (e.g. cholic acid 7-sulfate or derivative thereof), allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. The term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

In some embodiments, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs. In addition to the compound of any of Formulas (I)-(XVII), the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the compound of any of Formula (I)-(XVII), or Formula (I′)-(XVII′), are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The compound of any of Formula (I)-(XVII) or Formula (I′)-(XVII′) can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the compound of any of Formula (I)-(XVII) or Formula (I′)-(XVII′) can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

As used herein, the term “restricts delivery of the composition to the gastrointestinal tract” refers to a formulation that permits or facilitates the delivery of the agent or pharmaceutical composition described herein to the colon, large intestine, or small intestine in viable form. Enteric coating or micro- or nano-particle formulations can facilitate such delivery as can, for example, buffer or other protective formulations.

In some embodiments, the carrier or excipient restricts delivery of the composition to the gastrointestinal tract. In some embodiments, the composition provided herein is restricted to the gastrointestinal tract by the addition of a sulfate group or a polar group to the compounds.

In some embodiments, the carrier or excipient is an enteric coating or enteric-coated drug delivery device. As used herein, the terms “enteric coating” or “enteric-coated drug delivery device” refers to any drug delivery method that can be administered orally but is not degraded or activated until the device enters the intestines. Such methods can utilize a coating or encapsulation that is degraded using e.g., pH dependent means, permitting protection of the delivery device and the agent to be administered or transplanted throughout the gastrointestinal tract until the device reaches the alkaline pH of the intestines (e.g. cecum or colon).

An enteric coating can control the location of where an agent is released in the digestive system. Thus, an enteric coating can be used such that a pharmaceutical composition does not dissolve and release the agent in the stomach, but rather travels to the intestine, where it dissolves and releases the agent in an environment that is most beneficial for increasing GLP-1 secretion (e.g. targeting L-cells located in the cecum, ileum, large intestine, or colon). An enteric coating can be stable at low pH (such as in the stomach) and can dissolve at higher pH (for example, in the intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade names AQUACOAT™, AQUACOAT ECD™, SEPIFILM™, KLUCEL™, and METOLOSE™); polyvinylacetate phthalate (trade name SURETERIC™); and methacrylic acid (trade names EUDRAGIT™, EUDRAGIT L 100-55™ from Evonik Industries, Germany).

Another example of methods known in the art that allow for restriction of pharmaceutical compositions to the intestines, include enteric magnesium micromotors (EMgMs). EMgMs are described in the art, for example, in Li et al. ACS NANO, (2016).

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

### Dosing

The term “effective amount” is used interchangeably with the term “therapeutically effective amount” or “amount sufficient” and refers to the amount of at least one agonist of TGR5 or the VDR e.g., cholic acid 7-sulfate of a pharmaceutical composition, at dosages and for periods of time necessary to achieve the desired therapeutic result, for example, to “attenuate”, reduce or stop at least one symptom of diabetes, obesity, or an inflammatory disease. For example, an effective amount using the methods as disclosed herein would be considered as the amount sufficient to reduce one or more symptoms of diabetes, obesity, or an inflammatory disease by at least 10%. An effective amount as used herein would also include an amount sufficient to prevent or delay the development of such a symptom, alter the course of a symptom disease (for example but not limited to, slow the progression of a symptom of the disease), or reverse a symptom of the disease in a subject suffering from diabetes, prediabetes, hyperglycemia, obesity, or an inflammatory disease. Accordingly, the term “effective amount” or “therapeutically effective amount” as used herein refers to the amount of therapeutic agent (e.g. cholic acid 7-sulfate) of a pharmaceutical composition to alleviate at least one symptom of a disease. Stated another way, “therapeutically effective amount” of an agonist of TGR5 or the VDR as disclosed herein is the amount of an agonist which exerts a beneficial effect on, for example, the symptoms of the disease (e.g. diabetes). The dosage administered, as single or multiple doses, to an individual will vary depending upon a variety of factors, including pharmacokinetic properties of the inhibitor, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, the phrases “therapeutically-effective” and “effective for the treatment, prevention, or inhibition”, are intended to qualify agonist as disclosed herein which will achieve the goal of reduction in the severity of a diabetes, obesity, or an inflammatory disease or at one related symptom thereof.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animals. Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The compounds described herein can be administered at once, or can be divided into a number of smaller doses to be administered at intervals of time. It is understood that the precise dosage and duration of treatment will be a function of the location of where the composition is parenterally administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

In one embodiment of any of the aspects, the agent or composition is administered continuously (e.g., at constant levels over a period of time). Continuous administration of an agent can be achieved, e.g., by epidermal patches, continuous release formulations, or on-body injectors.

The compound can be administered as a single bolus or multiple boluses, as a continuous infusion, or a combination thereof. For example, the compound can be administered as a single bolus initially, and then administered as a continuous infusion following the bolus. The rate of the infusion can be any desired rate. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

The terms “co-administration” or the like, as used herein, are meant to encompass administration of the selected therapeutic agents to a single patient and are intended to include treatment regimens in which the agents are administered by the same or different route of administration or at the same or different time.

“Unit dosage form” as the term is used herein refers to a dosage for suitable one administration. By way of example a unit dosage form can be an amount of therapeutic disposed in a delivery device, e.g., a syringe or intravenous drip bag. In one embodiment of any of the aspects, a unit dosage form is administered in a single administration. In another embodiment of any of the aspects, more than one-unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further agents, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

In one embodiment of any of the aspects, the agent or compositions described herein are used as a monotherapy. In another embodiment of any of the aspects, the agents described herein can be used in combination with other known agents and therapies for diabetes. Administered “in combination,” as used herein, means that two (or more) different treatments are delivered to the subject during the course of the subject's affliction with the disorder, e.g., the two or more treatments are delivered after the subject has been diagnosed with the disorder (e.g. diabetes) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.”

In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The compounds and agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics currently used to treat or prevent diabetes include, but are not limited to, insulin therapy, sulfonylureas (e.g. glyburide), meglitinides (e.g. nataglinide), SGLT2 inhibitors (e.g. canaglifozin), bile acid sequesterants (e.g. colesevelam), dopamine-2-agonists (e.g. bromocriptine), biguanides (e.g. metformin), DPP-4 inhibitors (e.g. alogliptin, linagliptin, etc.), alpha-glucosidase inhibitors (e.g. acarbose and miglitol), thiazolidinediones (e.g. rosiglitazone), and other treatments for diabetes known in the art.

When administered in combination, the agent or composition and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of diabetes) is lower (e.g., at least 20%, at least 30%, at least 40%, or at least 50% lower) than the amount or dosage of each agent individually required to achieve the same therapeutic effect.

### Administration

In some embodiments of any of the aspects, the agent is administered by direct injection, subcutaneous injection, muscular injection, oral, or nasal administration. In some embodiments, the administering of the agent or pharmaceutical composition provided herein reduces glucose levels in the serum of a subject.

The terms “administered” and “subjected” are used interchangeably in the context of treatment of a disease or disorder.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered orally. In some embodiments, the agents or compositions provided herein are directly injected into the portal vein. For example, injection into the portal vein can limit systemic side effects of the agent or pharmaceutical composition. In some embodiments, the compositions provided herein are implanted into the portal vein for sustained release. In some embodiments, the compositions are administered via an injection port.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. Without limitations, oral administration can be in the form of solutions, suspensions, tablets, pills, capsules, sustained-release formulations, oral rinses, powders and the like.

Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments of any of the aspects, described herein is an agent or pharmaceutical composition that is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of diabetes, can be determined by the skilled practitioner. However, a treatment is considered “effective treatment,” as the term is used herein, if one or more of the signs or symptoms of diabetes, obesity, or an inflammatory disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., glucose levels or glucose tolerance. Efficacy can also be measured by a failure of an individual to worsen as assessed by hospitalization, or need for medical interventions (i.e., progression of the symptoms). Methods of measuring these indicators are known to those of skill in the art and/or are described herein.

Efficacy can be assessed in animal models of a condition described herein, for example, a mouse model or an appropriate animal model of diabetes, as the case may be. When using an experimental animal model, efficacy of treatment is evidenced when a statistically significant change in a marker is observed, e.g., reduced blood glucose levels.

### Genetically Engineered Microorganisms

In one aspect of any of the embodiments, provided herein is a method for treating diabetes, obesity, or an inflammatory disease, the method comprising: administering to a subject in need thereof a genetically engineered microorganism or population thereof, that expresses an agent that increases levels or activity of cholic acid 7-sulfate.

In another aspect of any of the embodiments, provided herein is a method for treating diabetes, obesity, or an inflammatory disease, the method comprising: administering to a subject in need thereof a genetically engineered microorganism or population thereof, that secretes cholic acid 7-sulfate.

The term “microorganism” as used herein refers to any microscopic-organism, matter, or component that is derived, originated from, or secreted by a microbe. Non-limiting examples of microorganisms include viruses, prokaryotic organisms (e.g. bacterium), or eukaryotic organisms (e.g. yeast, fungus, etc.).

The term “genetically engineered microorganism” as used herein refers to a microorganism that has been transformed by a small molecule, gene editing system, vector, plasmid, DNA, RNA, microRNA, lipoproteins, polypeptides, or the like to alter their functional properties (e.g. secrete cholic acid 7-sulfate). Examples of methods and compositions related to genetically engineered microorganisms are known in the art such as U.S. Pat. Nos. 7,354,592B2, 4,190,495A, 6,015,703A, US20080038805A1, and U.S. Pat. No. 5,733,540A, the contents of which are all incorporated by reference herein in their entireties.

In some embodiments of any of the aspects, the genetically engineered microorganism is a bacterium. In some embodiments, the bacterium is one that is found in the gastrointestinal tract. Exemplary bacteria include, but are not limited to Lactobacillus, Escherichia, Proteobacteria, Actinobacteria, Firmicutes, Bacteroidetes, Saccharomyces, Bifidobacterium, Faecalibacterium, Prevotella, Ruminococcus, Bacteroides their species, or any other bacteria known in the art. The bacteria can be genetically modified using methods known in the art (e.g. molecular cloning) to increase sulfation of cholic acid or secrete cholic acid 7-sulfate or derivative thereof in the gastrointestinal tract.

### Selected Definitions

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are provided herein.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

As used herein, the term “modulates” refers to an effect including increasing or decreasing a given parameter as those terms are defined herein.

As used herein, the term “contacting” when used in reference to a cell or organ, encompasses both introducing or administering an agent, surface, hormone, etc. to the cell, tissue, or organ in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those provided herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

### EXAMPLES

### Example 1: Cholic Acid 7-Sulfate

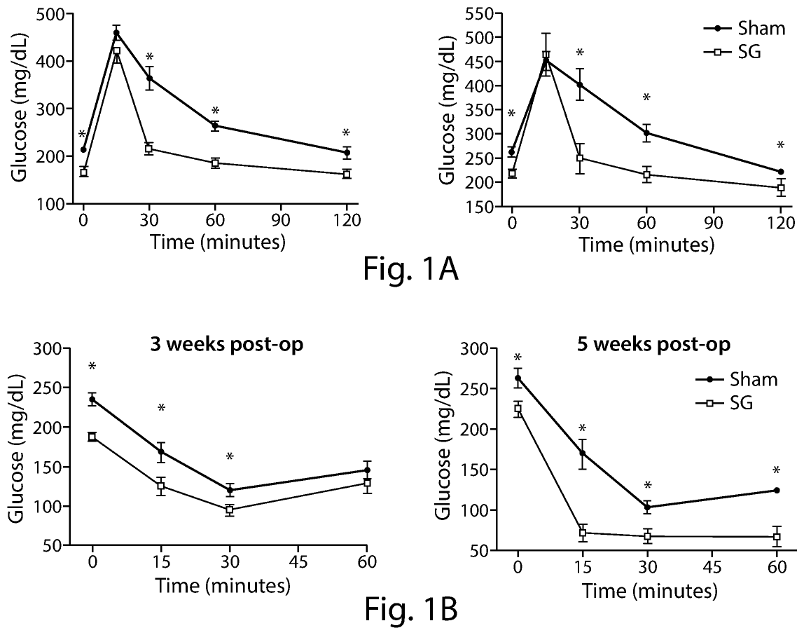

High fat diet-fed mice post-sleeve show improved glucose tolerance and insulin sensitivity (FIG. 1A-B) consistent with what has been observed before in humans. Therefore, the mouse model provided herein is used to study the amelioration of diabetic phenotypes post-sleeve surgery. Mice are suitable model for bariatric surgery-induced amelioration of diabetic phenotypes.

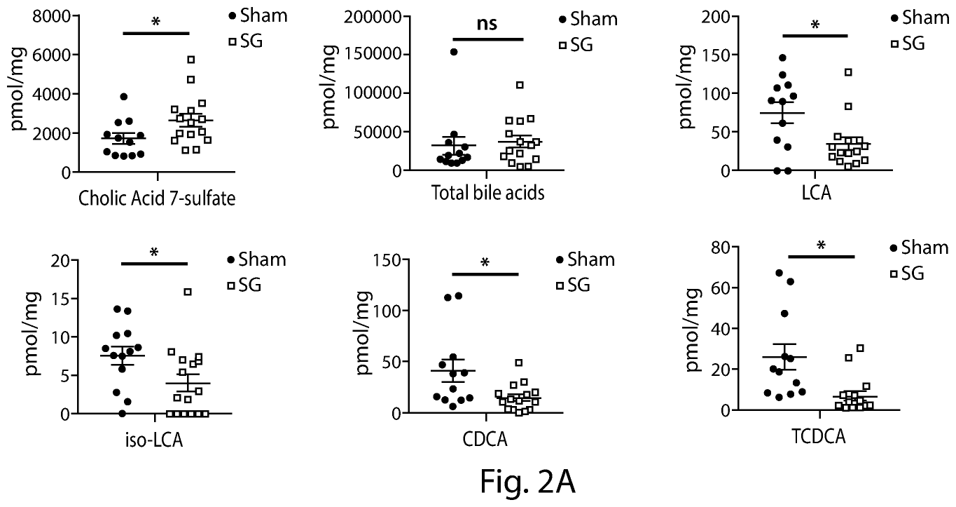

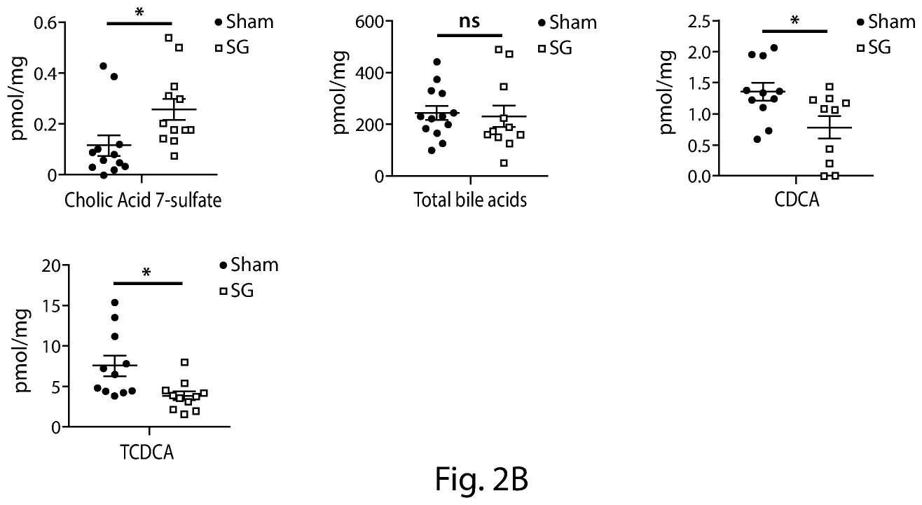

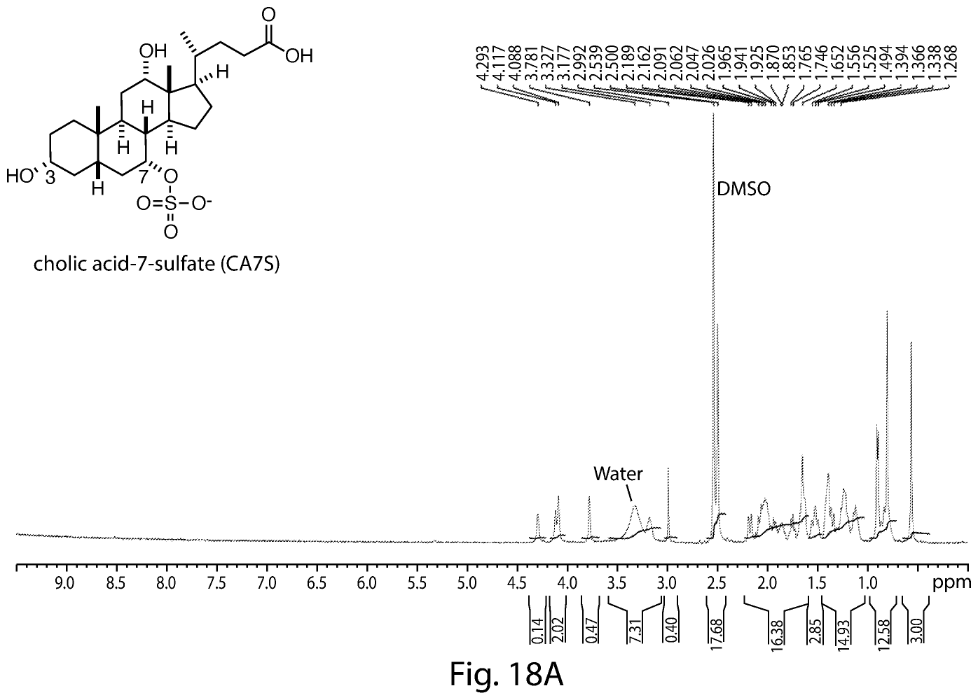

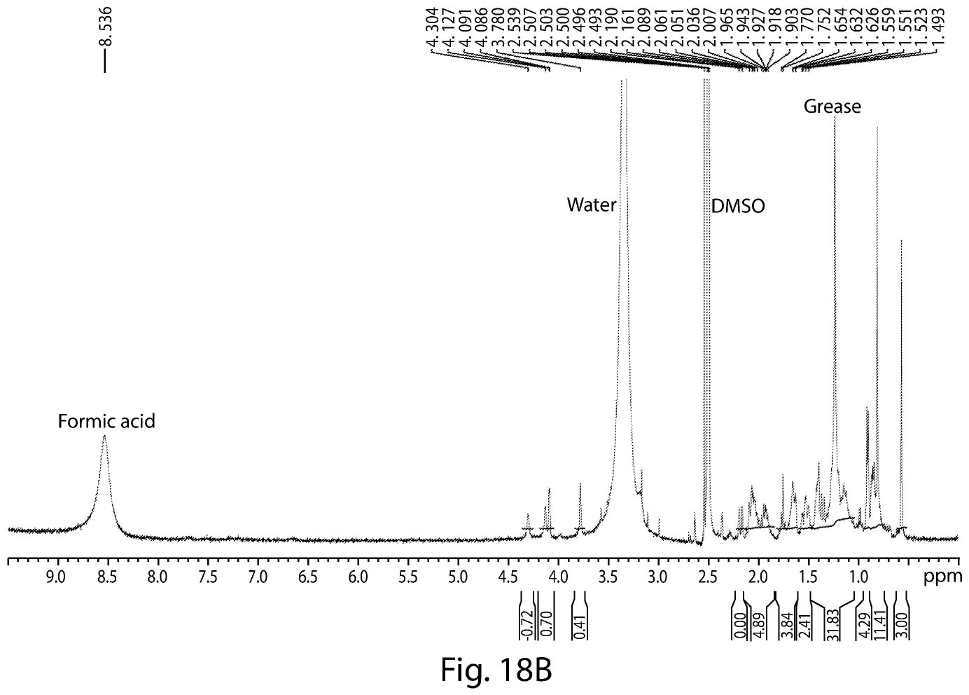

Bile acid profiling was performed and revealed significant changes in individual bile acids in mice post-sleeve. Interestingly, mice 6 weeks post-sleeve have higher levels of cholic acid 7-sulfate in their cecum compared to sham-operated mice (FIG. 2A). It was confirmed that the molecule in the bile acid was cholic acid 7-sulfate by NMR (FIG. 18A-B). Furthermore, mice post-sleeve have lower levels of secondary bile acid LCA and components of the “CDCA pathway” including CDCA, TCDCA, and iso-LCA in their cecum (FIG. 2A).

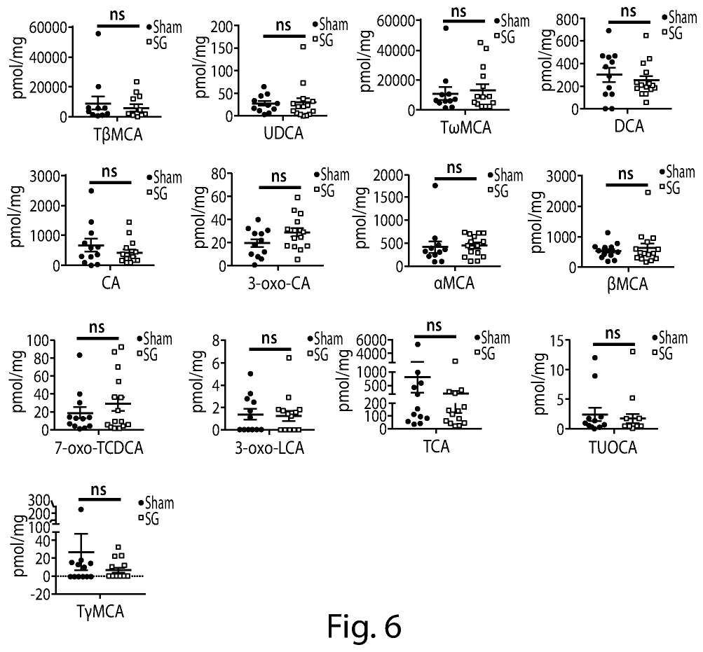

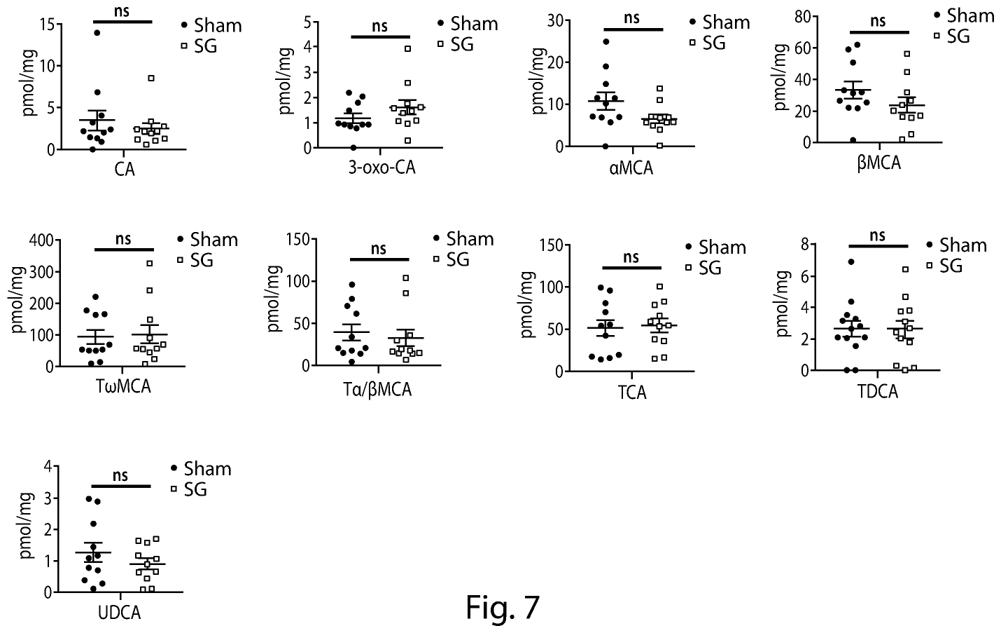

The total bile acids and other bile acids did not differ significantly in cecum of mice operated with sleeve or sham surgery (FIG. 6). Sleeve mice livers showed increased cholic acid 7-sulfate, CDCA, and TCDCA (FIG. 2B). However, total bile acids and other bile acids did not differ significantly in liver of mice operated with sleeve or sham surgery (FIG. 7).

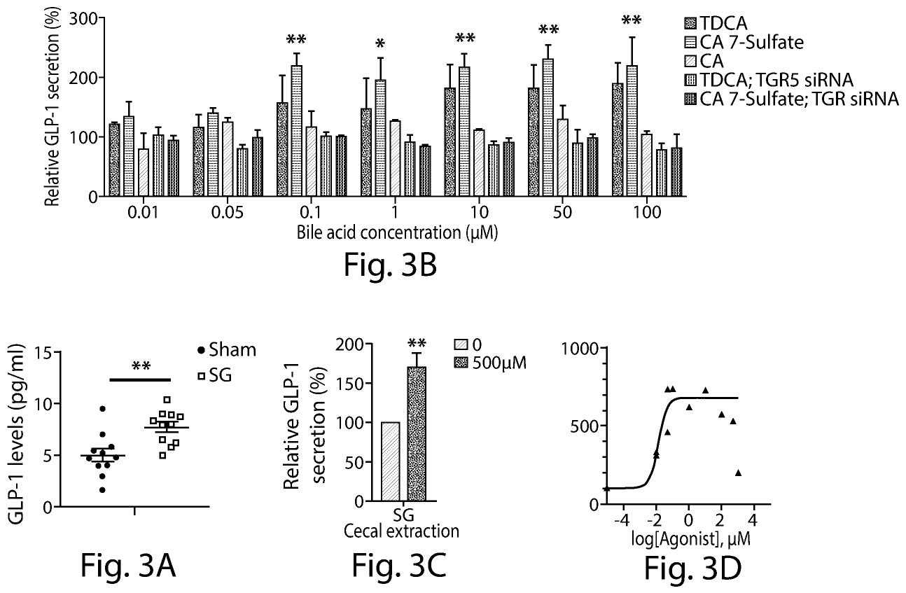

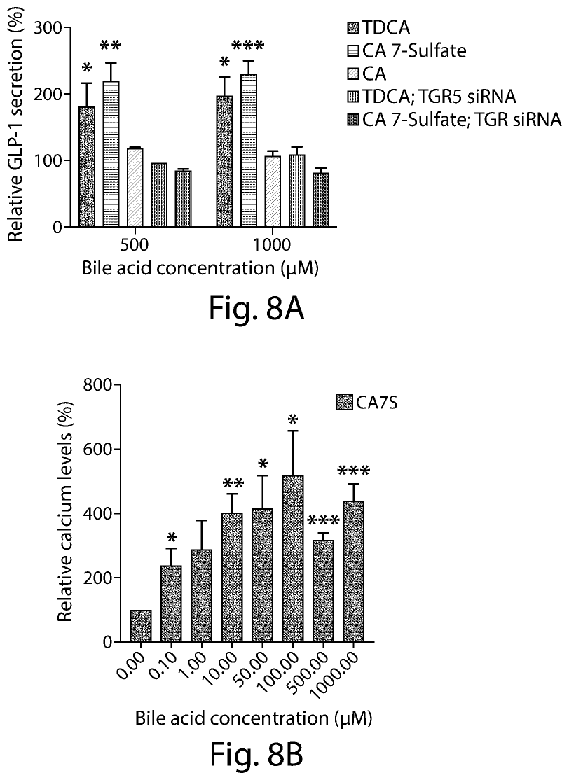

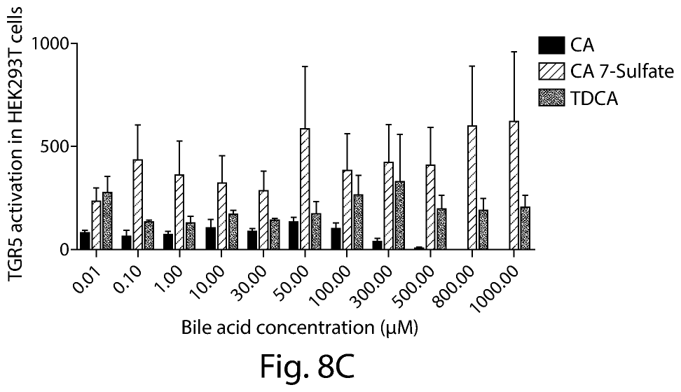

It was observed that sleeve mice show increase in GLP-1 in systemic circulation (FIG. 3A). Cholic acid 7-sulfate induces GLP-1 secretion in vitro better than the known GLP-1 inducer TDCA, while cholic acid had no effect (FIG. 3B and FIG. 8).

To identify a particular target of cholic acid 7-sulfate, it was discovered that cholic acid 7-sulfate-mediated induction of GLP-1 and requires TGR5. This was confirmed when knockdown of TGR5 abolished GLP-1 secretion (FIG. 3B and FIG. 8A). Therefore, cholic acid 7-sulfate is a TGR5 agonist and induces GLP-1 secretion in vitro.

To further investigate this mechanism, cholic acid 7-sulfate was extracted from cecum of mice and found to also exhibit activity inducing GLP-1 secretion in vitro (FIG. 3C). Cholic acid 7-sulfate activates TGR5 in L-cells, dose response curve shows an EC50 of 0.013 μM (FIG. 3D). Cholic acid 7-sulfate increased calcium levels in L-cells in vitro (FIG. 8B). Cholic acid 7-sulfate induces TGR5 activation in HEK293T cells (FIG. 8C).

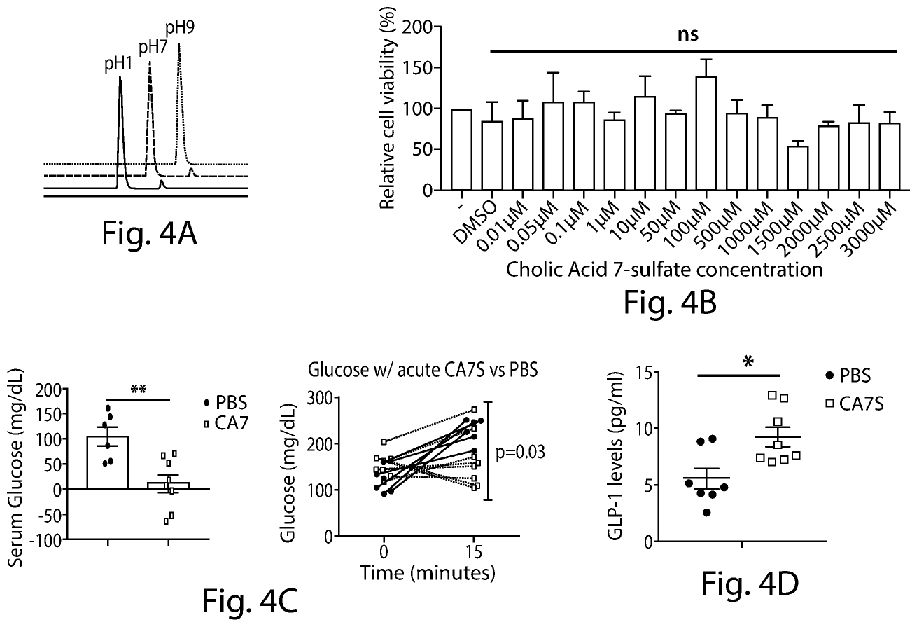

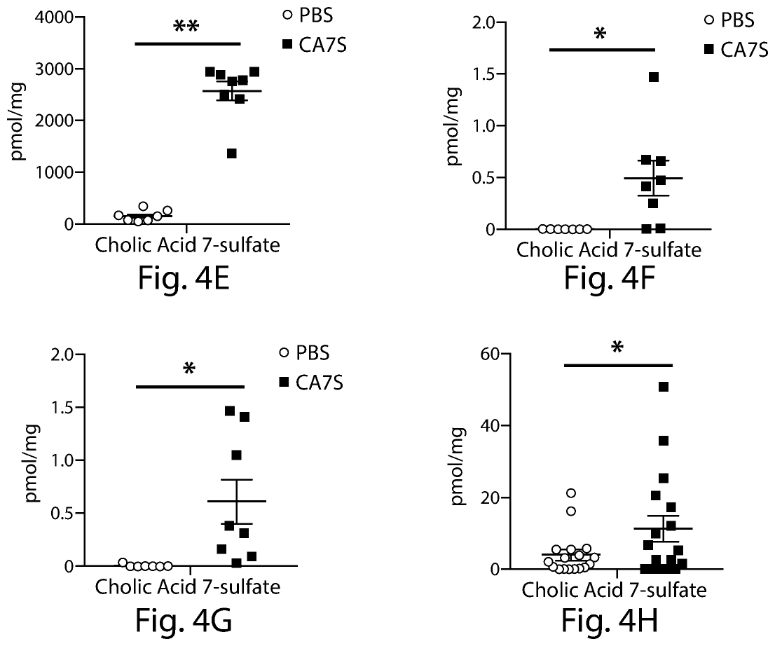

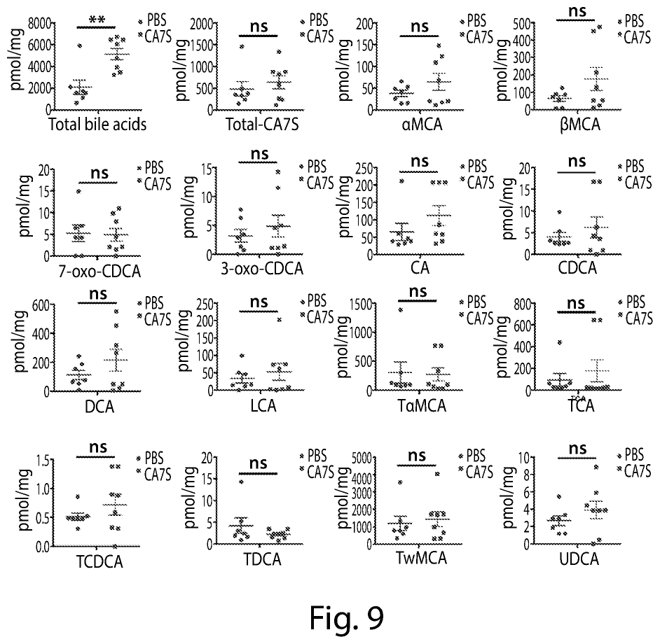

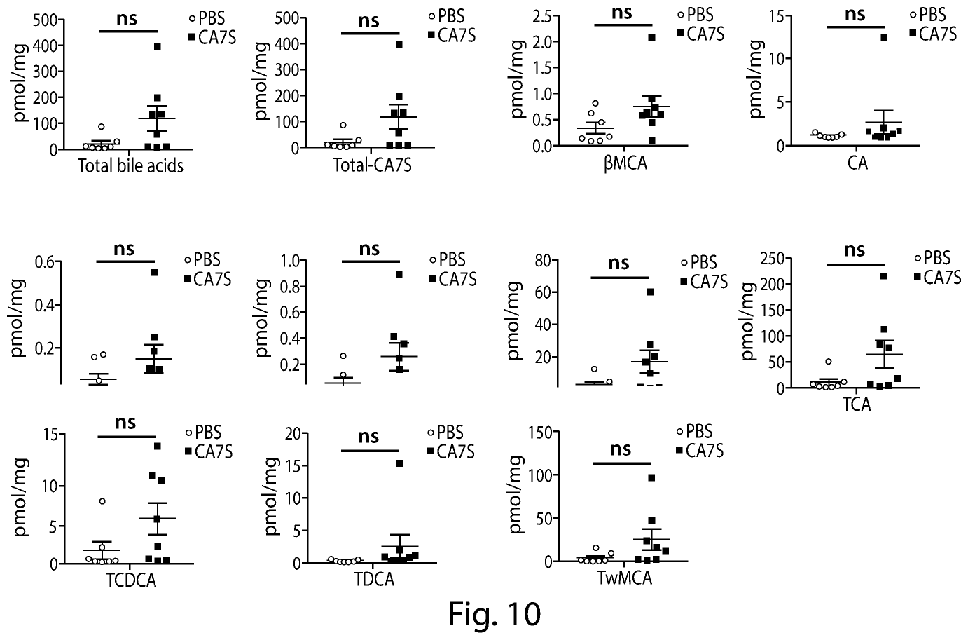

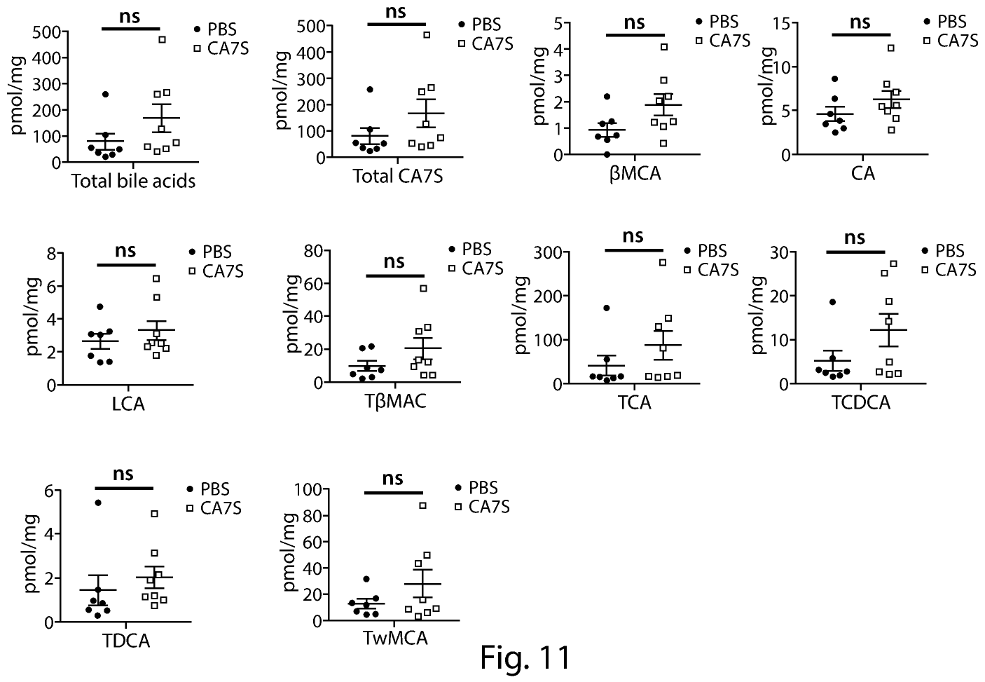

Cholic acid 7-sulfate is stable in a wide range of pHs, and has no toxicity in intestinal Caco cells in vitro (FIG. 4A-B). Treatment of HFD-fed mice with cholic acid 7-sulfate in vivo reduced blood glucose levels and induced GLP-1 levels within 15 min. of treatment (FIG. 4C-D). Therefore, acute cholic acid 7-sulfate treatment induces GLP-1 and reduces serum glucose levels in vivo. Dosing with 1 mg cholic acid 7-sulfate resulted in ˜2500 μM cholic acid 7-sulfate in the cecum, similar to the amounts that were observed in sleeve-operated mice (FIG. 4E). Ectopic introduction of cholic acid 7-sulfate allowed only minor amounts to leak into systemic circulation and in the portal vein, and did not significantly affect other bile acids in the cecum, blood, or the portal vein (FIG. 4F-G, FIG. 9-11). Feces from human patients pre- and post-sleeve gastrectomy also have an increase in cholic acid 7-sulfate (FIG. 4H).

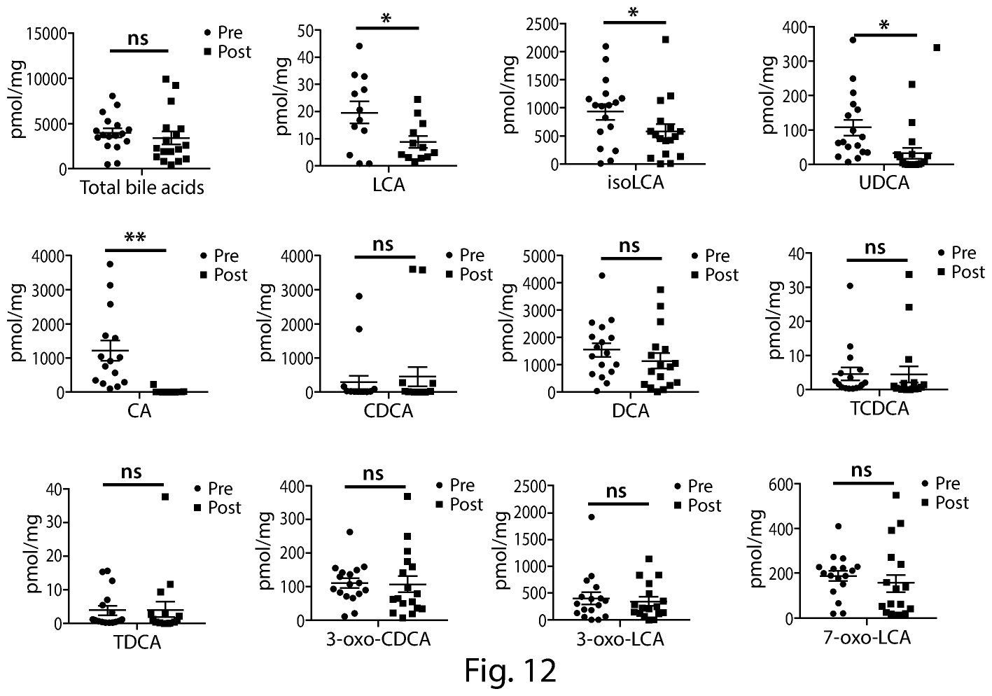

Interestingly, human fecal samples post-sleeve exhibit a reduction in levels of secondary bile acids LCA, iso-LCA, and UDCA, similar to what was observed in mice post-sleeve (FIG. 12). Other bile acids and total bile acids were not significantly affected, except for calcium levels. (FIG. 12).

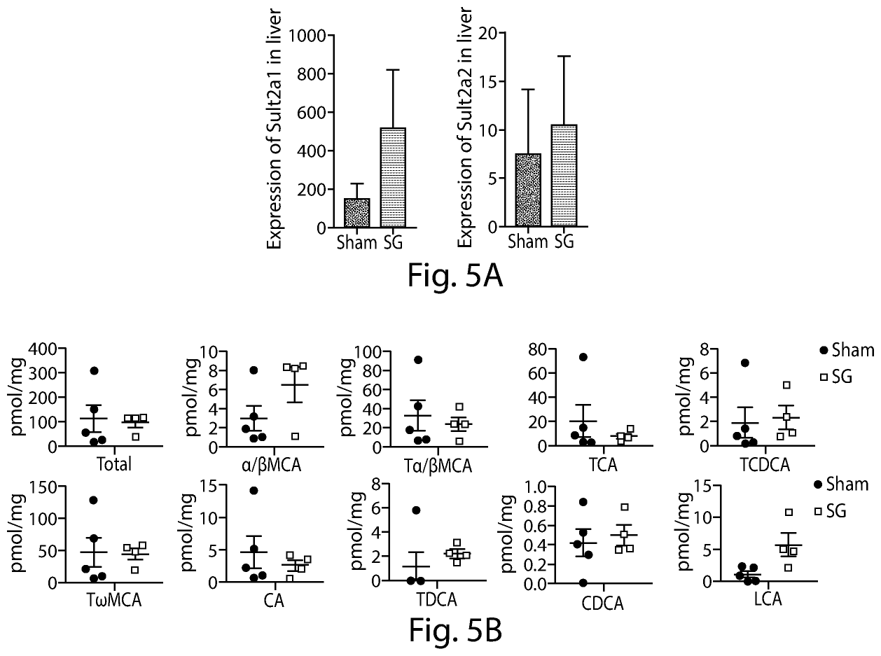

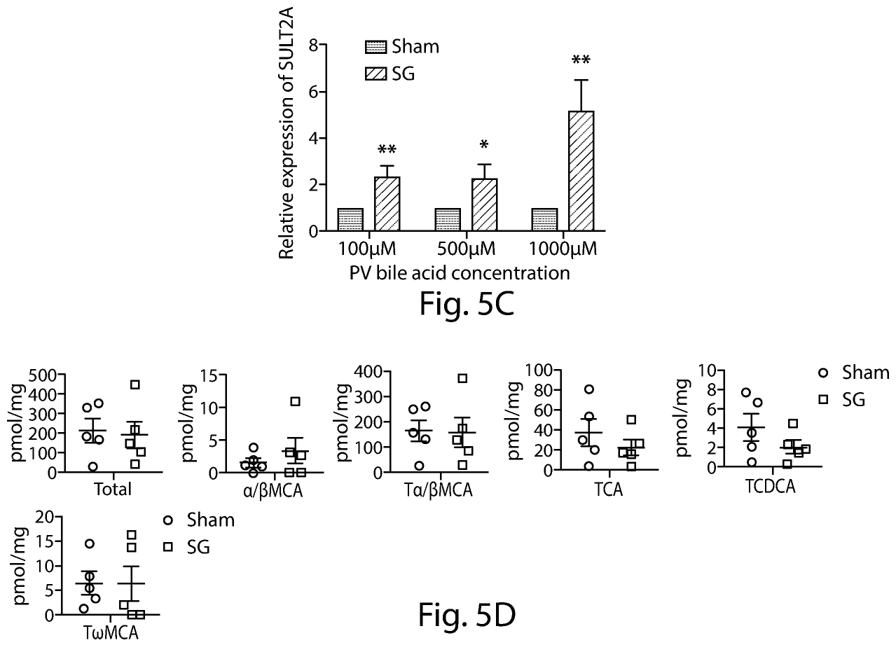

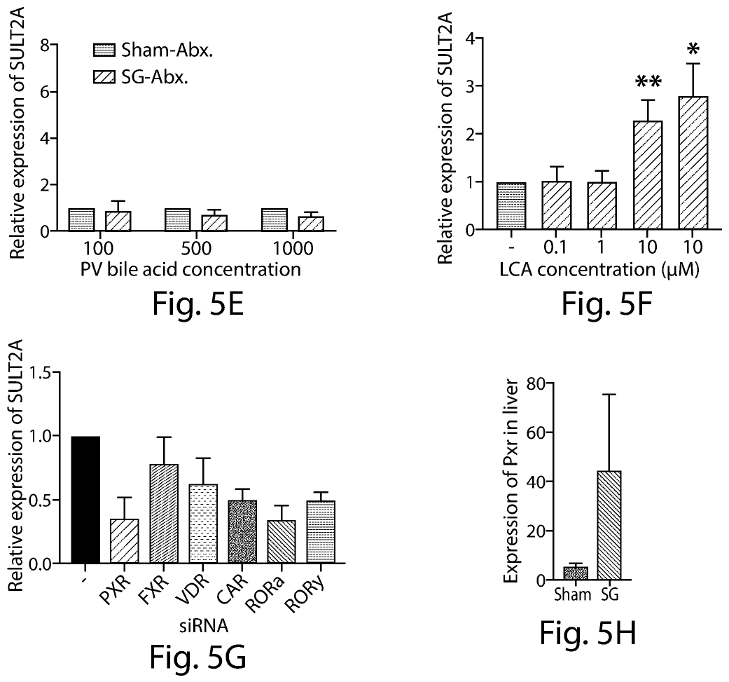

Mice livers show an increase in SULT2A enzyme isoform 1, previously shown to sulfate bile acids (FIG. 5A).

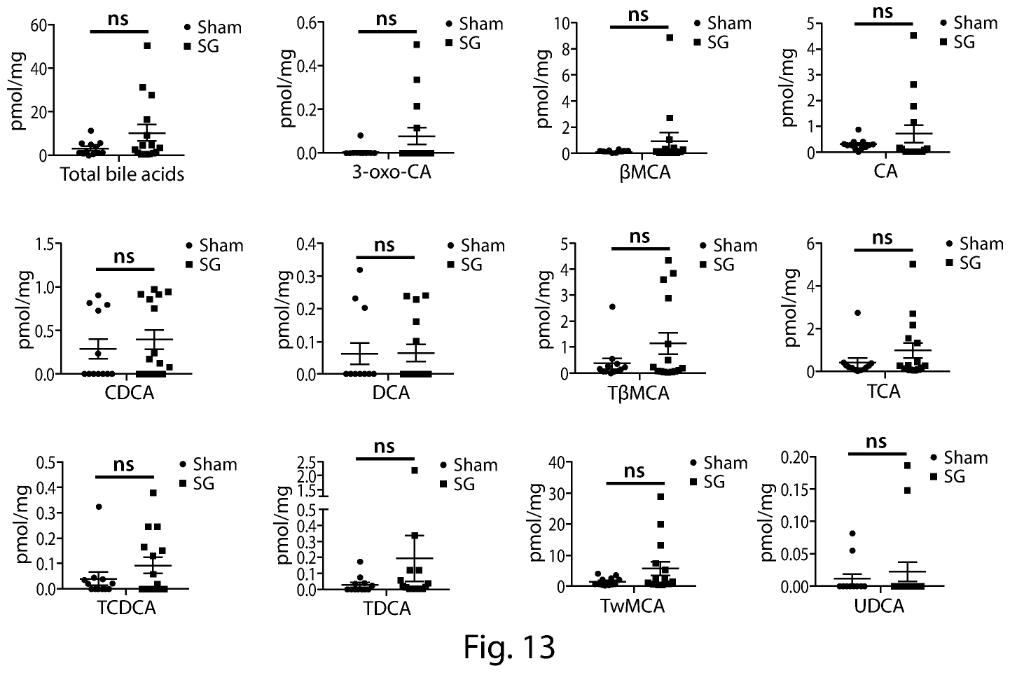

Sulfation is a detoxification method to excrete toxic bile acids. Bile acids have been shown to tightly regulate their own synthesis, conjugation, and sulfation. The liver is the major site for synthesis and sulfation of bile acids, therefore bile acids in the hepatic portal vein were analyzed to determine the origin of sulfated cholic acid and a mechanism for the increase in cholic acid 7-sulfate in sleeve mice. The hepatic portal vein is part of the enterohepatic circulation of bile acids. The liver receives 80% of its blood from the hepatic portal vein. The portal vein has a different repertoire of bile acids compared to circulating blood (FIG. 5B & FIG. 13).

To not be bound by a particular theory, it was hypothesized that bile acids in the hepatic portal vein signal in the liver to induce sulfation of cholic acid. Pools of bile acids were tested mimicking those observed in the sleeve- and sham-operated mouse portal veins in inducing SULT2A1 in vitro. Using HepG2 cells, it was observed that the bile acid pool in the portal vein of sleeve-operated mice significantly induced SULT2A1 compared to the portal vein bile acid pool in sham-operated mice (FIG. 5C)

Bile acids are modified in the intestine by the microbiome. Therefore, the influence of the microbiome in inducing sulfation of bile acids in the liver was tested. Sleeve gastrectomy was performed and sham surgery on HFD-fed mice treated with antibiotics. Pools of bile acids mimicking those observed in the antibiotic-treated sleeve- and sham-operated mouse portal veins were tested inducing SULT2A1 in HepG2 cells. there was no difference in induction of SULT2A1 between the pools observed (FIG. 5D-E).

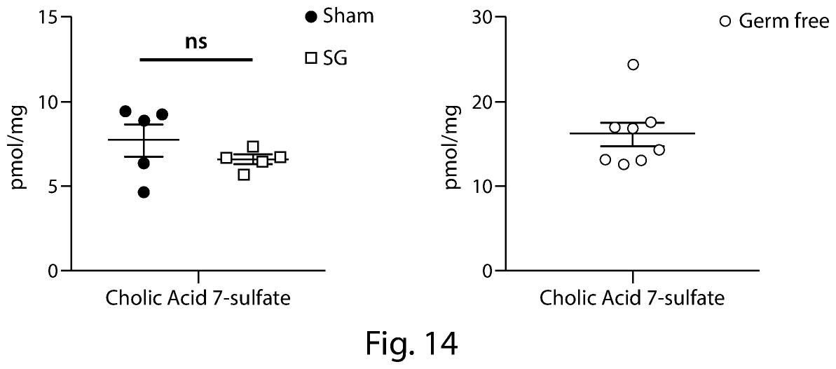

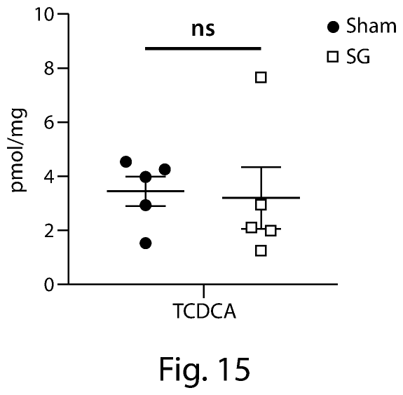

Consistently, it was observed that there was not cholic acid 7-sulfate in the liver and approximately 200-fold lower levels of cholic acid 7-sulfate in the cecum in antibiotic-treated mice (FIG. 13 and FIG. 14) compared to HFD-fed conventional mice. Also, there was no significant difference in cholic acid 7-sulfate levels between antibiotic-treated sleeve- and sham-operated mouse cecum (FIG. 8). This suggests that a microbiome is required for sulfation of cholic acid. In support of this hypothesis, germ-free animals fed a high fat diet also show 200-fold lower cholic acid 7-sulfate in their cecum (FIG. 8).

To test which bile acid(s) may be involved in inducing SULT2A1 enzyme, the bile acids in the portal vein that were significantly different between HFD-fed conventional mice and HFD-fed mice treated with antibiotics were analyzed. It was observed that LCA, TDCA, CA, and CDCA were absent in the antibiotic-treated mouse portal veins (FIG. 5D).

Amongst these, LCA induced SULT2A1 in HepG2, while others did not in all concentrations tested (FIG. 5F). LCA levels were also increased in sleeve mice compared to sham-operated, while the total bile acid levels did not differ significantly, suggesting that LCA is an inducer of SULT2A1 expression (FIG. 5B). To identify the receptor involved in LCA-mediated induction of SULT2A1 in liver cells, siRNA of known receptors was performed. The PXR receptor was consistently upregulated in mice post-sleeve in the liver (FIG. 5G-H).

### Example 2: Bariatric Surgery Reveals a Gut-Restricted TGR5 Agonist and GLP-1 Secratogue

The molecular mechanisms underlying the near-immediate resolution of diabetic phenotypes following bariatric surgery remain largely unknown. Here, the data show that sleeve gastrectomy leads to an increase in a naturally occurring bile acid metabolite, cholic acid 7-sulfate. This metabolite is a gut-restricted TGR5 agonist that induces GLP-1 secretion and reduces blood glucose levels in a mouse model. Thus these studies reveal a molecular link between bariatric surgery and amelioration of diabetic phenotypes.

### Results

Obesity and type 2 diabetes (T2D) are medical pandemics. Bariatric surgery, in the form of Roux-en-Y gastric bypass or sleeve gastrectomy (SG), is currently the most effective and durable treatment for obesity and related comorbidities^{1,2}. Owing to robust post-surgical metabolic benefits and favorable side-effect profile, SG is the most common bariatric surgery performed in the US ³. While maximal weight loss occurs at 1 year, many patients see resolution of their T2D within days of surgery⁴. For a majority of patients, remission is durable, lasting for at least 7 years^{1,4}. The molecular mechanisms underlying T2D remission, however, remain largely unknown⁵.

Two consistently observed post-surgical changes are increased levels of GLP-1, a circulating incretin hormone, and changes in the systemic repertoire of bile acids (BAs). BAs are cholesterol-derived metabolites that play crucial roles in host metabolism by acting as detergents that aid in the absorption of lipids and vitamins and as ligands for host receptors⁶. While the potential therapeutic benefits of GLP-1 have been recently explored⁷, the causal role of bile acids in mediating beneficial metabolic changes post-surgery remains unclear. Thus far, research efforts have focused on overall changes in the total BA pool or in conjugated and unconjugated BA forms following bariatric surgery^{8,9}. Individual BAs, however, have different binding affinities for nuclear hormone receptors (NhRs) and GPCRs, and thus unique abilities to modulate glucose homeostasis, lipid accumulation, and energy expenditure^{6,10}. It is not sufficient, therefore, to limit analyses to whole classes of BAs. Levels of individual BAs pre- and post-SG were identified to investigate whether specific BAs could be causally linked to changes in metabolic outcomes.

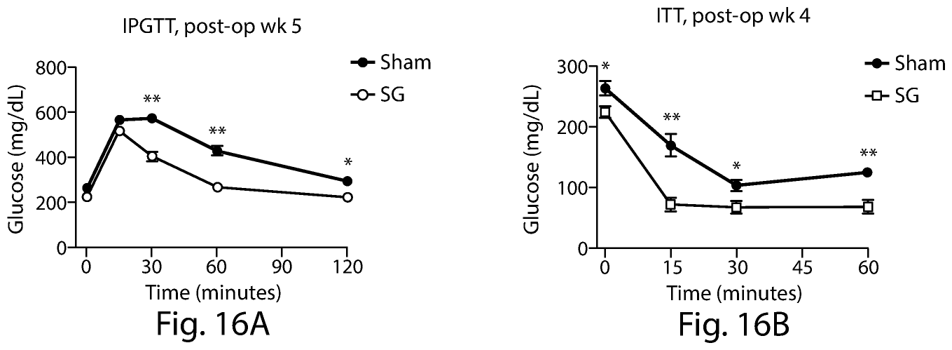

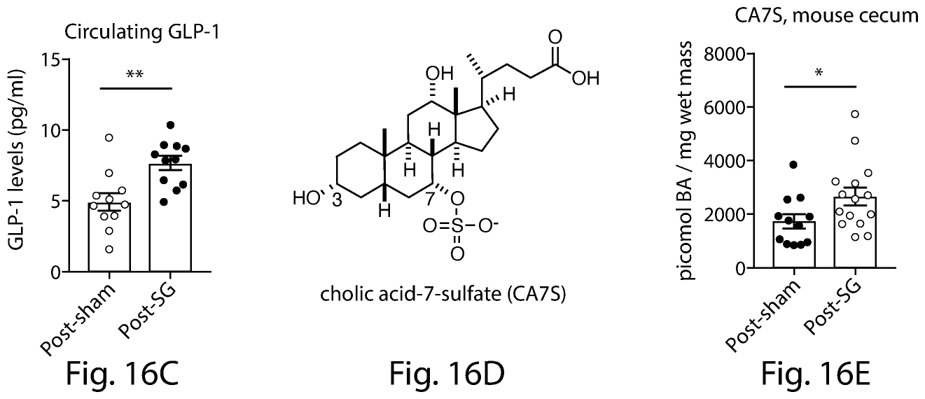

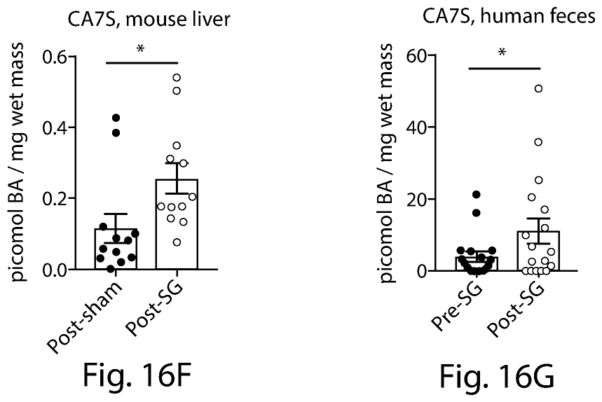

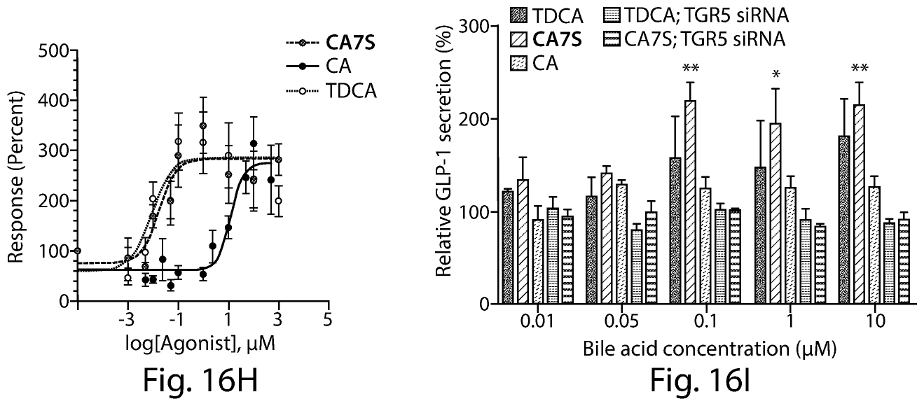

Rodent SG models mimic the positive metabolic outcomes observed in humans and are thus suitable for studying post-surgical outcomes¹¹. In this study, SG or sham surgery was performed on insulin-resistant, diet-induced obese (DIO) mice. SG mice displayed improved glucose tolerance and insulin sensitivity 4-5 weeks post-surgery compared to shams (FIG. 16A-B). Mice were euthanized six weeks post SG or sham surgery and their tissues were harvested. Consistent with studies involving human patients⁸, an increase in circulating GLP-1 in SG mice was observed (FIG. 16C). GLP-1 is secreted post-prandially by L-cells in the lower intestine and directly stimulates pancreatic insulin release⁷. Low levels of GLP-1 are associated with T2D, whereas increased levels post-SG correlate with weight-loss and T2D remission^{12,13}. Activation of TGR5, a G-protein coupled receptor (GPCR) with a primary role in energy metabolism, stimulates GLP-1 secretion¹⁴. Notably, gluco-regulatory benefits of SG are attenuated in TGR5−/− mice, demonstrating the important role of this receptor in mediating the anti-diabetic effects of SG¹⁵.

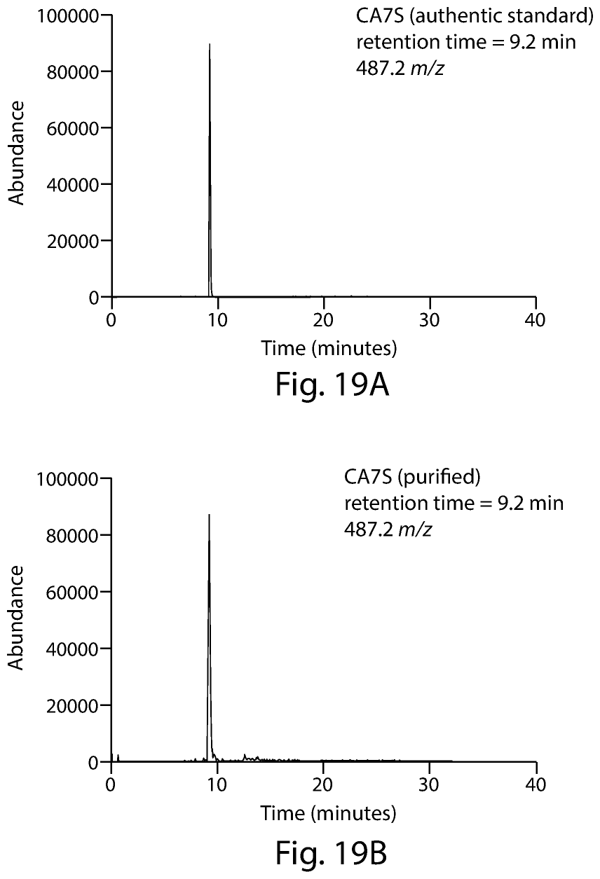

Individual BAs that are known agonists of TGR5 have been shown to induce GLP-1 secretion in lower-intestinal L-cells^{8,14}. Next, individual Bas were assayed in cecal contents of SG and sham mice using UPLC-MS. A significant increase in a monosulfated, trihydroxy bile acid in cecal contents of SG mice were observed. Using NMR spectroscopy, this compound was identified as cholic acid 7-sulfate (CA7S) (FIG. 16D-E, FIGS. 18-19). This molecule is a sulfated metabolite of cholic acid (CA), an abundant primary bile acid in both mice and humans. Sulfation of bile acids predominantly occurs in the liver¹⁶. Consistent with this observation, increased levels of CA7S were observed in the liver of SG mice (FIG. 16F). Notably, CA7S was the only bile acid detected whose levels were significantly higher in SG mouse cecal contents.

To determine the clinical relevance of this finding, BAs were assayed in stool from human patients who had undergone SG. Remarkably, fecal CA7S levels were also significantly increased in patients six months post-SG compared to their pre-surgery levels (FIG. 16G). This is the first report of a specific BA metabolite that is significantly increased following SG in both mice and human subjects.

Next, it was assessed whether CA7S is causally involved in the development of post-SG metabolic phenotypes, and in particular, GLP-1 secretion. Previous work has shown that sulfation of both natural BAs and synthetic analogs significantly alters the TGR5 agonistic activity of these compounds¹⁷. To not be bound by a particular theory, it was hypothesized that CA7S might possess altered TGR5 agonism compared to CA. The activation of human TGR5 in HEK293T cells by CA7S, CA, or tauro-deoxycholic acid (TDCA), a naturally occurring BA and potent TGR5 agonist¹⁸ were examined. CA7S activated human TGR5 in a dose-dependent manner and to a similar extent as TDCA. CA7S also displayed a lower EC50 (0.17 μM) than CA (12.22 μM) (FIG. 16H).

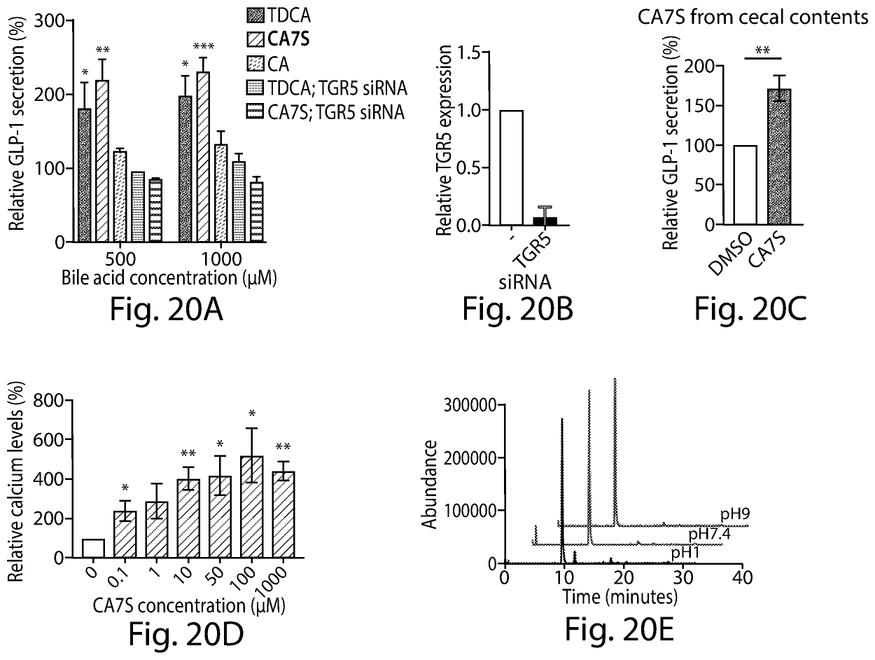

TDCA is currently one of the most potent naturally occurring GLP-1 secretagogue known¹⁸. It was observed that CA7S induced GLP-1 secretion to a similar degree as TDCA in a dose-dependent manner, while CA had no effect on GLP-1 secretion (FIG. 16I and FIG. 20A-B). CA7S extracted directly from cecal contents of SG mice also induced GLP-1 secretion in vitro (FIG. 20C). Furthermore, siRNA-mediated knockdown of TGR5 abolished both CA7S- and TDCA-mediated secretion of GLP-1 (FIG. 16I and FIG. 20A-B). This result indicates that induction of GLP-1 secretion by CA7S requires TGR5. TGR5 agonism also results in elevated intracellular calcium levels¹⁹. Consistent with this previous finding, a dose-dependent increase in calcium levels in NCI-H716 cells treated with CA7S was observed (FIG. 20D). Taken together, these results demonstrate that CA7S, a naturally occurring bile acid metabolite, is a potent TGR5 agonist and GLP-1 secretagogue.

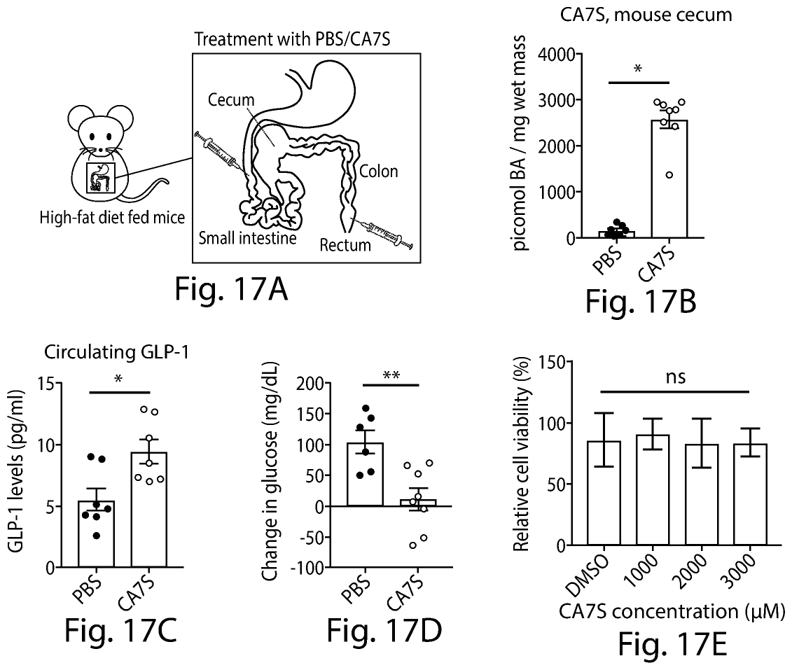

Next, the ability of CA7S to stimulate GLP-1 secretion and improve hyperglycemia in vivo was evaluated. DIO mice were treated with either CA7S or PBS via duodenal and rectal catheters (FIG. 17A). Administration of 1 mg of CA7S resulted in 2500 pmol/mg wet mass of CA7S on average in cecal contents, a concentration similar to observed post-SG levels (FIG. 17D, FIG. 17B, Table 1 below). Consistent with in vitro studies, CA7S-treated mice displayed increased systemic GLP-1 levels compared to PBS-treated mice within 15 minutes (FIG. 17C). Moreover, CA7S-treated mice exhibited reduced blood glucose levels compared to PBS-treated mice, suggesting that CA7S is protective against hyperglycemia (FIG. 17D).

TABLE 1
Cholic acid concentrations

| | | Cholic acid 7-sulfate | |
|---|---|---|---|
| Treatment | Tissue | concentration (mean ± SEM) | |
| HFD-fed mice; sham | Cecum | 1726 ± 267.1 | pmol/mg |
| surgery | Liver | 0.116 ± 0.04 | pmol/mg |
| | Hepatic portal vein | 0 ± 0 | pmol/mg |
| | Blood | 0 ± 0 | pmol/μl |
| HFD-fed mice; sleeve | Cecum | 2661 ± 331.3 | pmol/mg |
| gastrectomy | Liver | 0.2575 ± 0.04 | pmol/mg |
| | Hepatic portal vein | 0 ± 0 | pmol/mg |
| | Blood | 0 ± 0 | pmol/μl |
| HFD-fed mice; | Cecum | 161.1 ± 46.41 | pmol/mg |
| acute PBS treatment | Hepatic portal vein | 0.065 ± 0.056 | pmol/mg |
| | Blood | 0 ± 0 | pmol/μl |
| HFD-fed mice; acute | Cecum | 2577 ± 185.3 | pmol/mg |
| cholic acid 7-sulfate | Hepatic portal vein | 6.128 ± 2.111 | pmol/mg |
| treatment | Blood | 0.4954 ± 0.1673 | pmol/μl |

CA7S was undetectable in both circulating and portal venous blood from SG and sham-operated mice (Table 1). This result suggests that CA7S is neither recycled via enterohepatic circulation nor absorbed into systemic circulation. Ectopic introduction of CA7S resulted in only minor amounts in circulating and portal venous blood (Table 1). These findings are consistent with previous observations that sulfated BAs, in particular 7α-sulfated BAs, are poorly absorbed in the intestine¹⁶.

The results from this study may have clinical implications. While synthetic TGR5 agonists ameliorate diabetic phenotypes²⁰, their use as therapeutics is hampered by significant side effects. These compounds are absorbed into systemic circulation and can induce changes in the circulatory, digestive, and endocrine systems, causing changes in heart rate and blood pressure, induction of cholestasis, pancreatitis, and hepatic necrosis, and reduction in intestinal motility^{20,21}. Owing to these significant off-target effects, it has been suggested that an ideal TGR5-based therapeutic for T2D would specifically activate intestinal TGR5²¹. CA7S remains gut-restricted and is stable at physiological pHs (FIG. 20E). Furthermore, CA7S does not affect the viability of human intestine-derived Caco-2 cells at concentrations up to 3 mM (FIG. 17E). As a result of its beneficial metabolic effects, gut restriction, and low toxicity, CA7S could be a candidate for the development of a new T2D therapeutic. Further studies are required, however, to assess the long-term effects of this metabolite on glucose tolerance, insulin sensitivity, and weight in vivo. Nonetheless, through the identification of the TGR5 agonist CA7S, this work has uncovered a molecular connection between SG and the beneficial effects of this surgical intervention on metabolism.

### REFERENCES

Batterham, R. L. & Cummings, D. E. Mechanisms of Diabetes Improvement Following Bariatric/Metabolic Surgery. Diabetes Care 39, 893-901 (2016).
Gloy, V. L. et al. Bariatric surgery versus non-surgical treatment for obesity: a systematic review and meta-analysis of randomised controlled trials. BMJ 347, f5934-f5934 (2013).
Khorgami, Z. et al. Trends in utilization of bariatric surgery, 2010-2014: sleeve gastrectomy dominates. Surg Obes Relat Dis 13, 774-778 (2017).
Abbasi, J. Unveiling the ‘Magic’ of Diabetes Remission After Weight-Loss Surgery. JAMA 317, 571-574 (2017).
Ryan, K. K. et al. FXR is a molecular target for the effects of vertical sleeve gastrectomy. Nature 509, 183-188 (2014).
Fiorucci, S. & Distrutti, E. Bile Acid-Activated Receptors, Intestinal Microbiota, and the Treatment of Metabolic Disorders. Trends Mol Med 21, 702-714 (2015).
Madsbad, S. The role of glucagon-like peptide-1 impairment in obesity and potential therapeutic implications. Diabetes Obes Metab 16, 9-21 (2014).
Kaska, L., Sledzinski, T., Chomiczewska, A., Dettlaff-Pokora, A. & Swierczynski, J.

Improved glucose metabolism following bariatric surgery is associated with increased circulating bile acid concentrations and remodeling of the gut microbiome. World J. Gastroenterol. 22, 8698-8719 (2016).
Patti, M.-E. et al. Serum bile acids are higher in humans with prior gastric bypass: potential contribution to improved glucose and lipid metabolism. Obesity (Silver Spring) 17, 1671-1677 (2009).
Sayin, S. I. et al. Gut microbiota regulates bile acid metabolism by reducing the levels of tauro-beta-muricholic acid, a naturally occurring FXR antagonist. Cell Metab. 17, 225-235 (2013).
Lutz, T. A. & Bueter, M. The Use of Rat and Mouse Models in Bariatric Surgery Experiments. Front Nutr 3, 25 (2016).
Steinert, R. E., Beglinger, C. & Langhans, W. Intestinal GLP-1 and satiation: from man to rodents and back. Int J Obes 40, 198-205 (2015).
Lastya, A., Saraswati, M. R. & Suastika, K. The low level of glucagon-like peptide-1 (glp-1) is a risk factor of type 2 diabetes mellitus. BMC Res Notes 7, 849 (2014).
Duboc, H., Taché, Y. & Hofmann, A. F. The bile acid TGR5 membrane receptor: from basic research to clinical application. Dig Liver Dis 46, 302-312 (2014).
McGavigan, A. K. et al. TGR5 contributes to glucoregulatory improvements after vertical sleeve gastrectomy in mice. Gut 66, 226-234 (2017).
Alnouti, Y. Bile Acid sulfation: a pathway of bile acid elimination and detoxification. Toxicol. Sci. 108, 225-246 (2009).
Sato, H. et al. Novel Potent and Selective Bile Acid Derivatives as TGR5 Agonists: Biological Screening, Structure-Activity Relationships, and Molecular Modeling Studies. Journal of Medicinal Chemistry 51, 1831-1841 (2008).
Brighton, C. A. et al. Bile Acids Trigger GLP-1 Release Predominantly by Accessing Basolaterally Located G Protein-Coupled Bile Acid Receptors. Endocrinology 156, 3961-3970 (2015).
Kuhre, R. E. et al. Peptide production and secretion in GLUTag, NCI-H716, and STC-1 cells: a comparison to native L-cells. Journal of Molecular Endocrinology 56, 201-211 (2016).
Hodge, R. J. & Nunez, D. J. Therapeutic potential of Takeda-G-protein-receptor-5 (TGR5) agonists. Hope or hype? Diabetes Obes Metab 18, 439-443 (2016).
Cao, H. et al. Intestinally-targeted TGR5 agonists equipped with quaternary ammonium have an improved hypoglycemic effect and reduced gallbladder filling effect. Sci Rep 6, 28676 (2016).

### Example 3: SAR of Cholic Acid 7-Sulfate

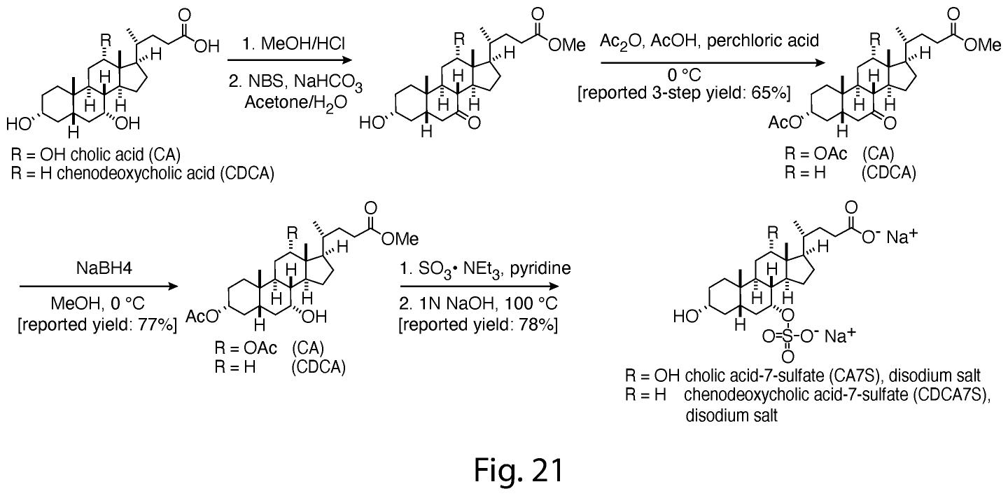

The synthesis of 7-sulfated bile acids are shown in FIG. 21. Synthesis of gram quantities (minimum of 2 grams, ideally to about 10 grams) of cholic acid 7-sulfate (CA7S) are shown.

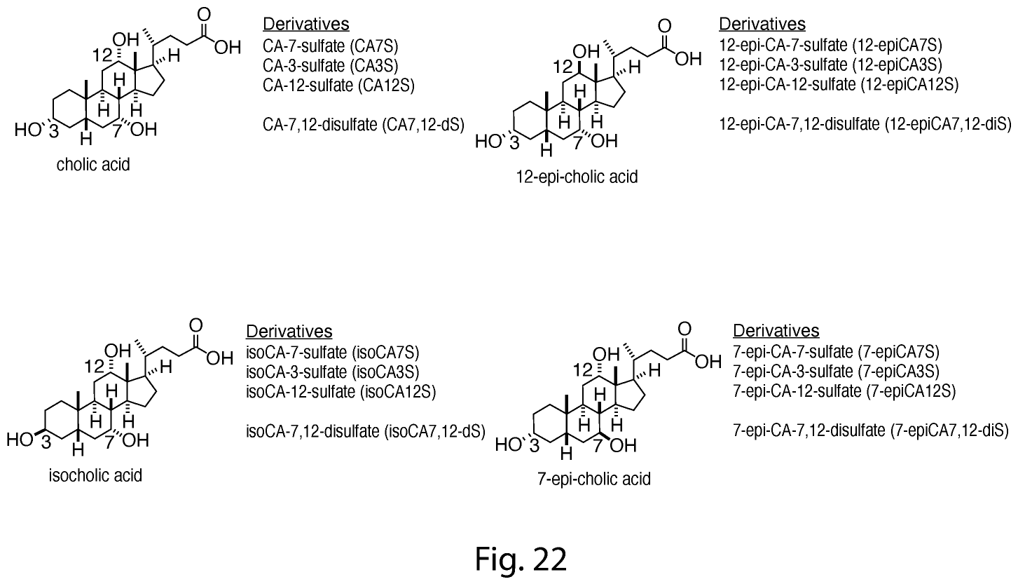

The synthesis of milligram quantities (about 100 mg each) of CA7S variants for structure-activity studies are shown in FIG. 22.

Many of these compounds are not commercially available. The goal of in vitro studies with these compounds is to determine the key structural elements that are necessary for TGR5 agonist activity (while attempting to maintain chemical properties that will GI-restrict the compound). The next step is the design and synthesis of non-natural derivatives. It is necessary to investigate the effect of combinations of bile acid cores and sulfate group(s) that can yield TGR5 agonists.

The syntheses of these compounds begin with the bile acid itself. One major limiting factor in which derivatives are accessible may be the availability and cost of the bile acid starting material. For example, cholic acid is cheap but the muricholic acids are expensive.

Lithocholic acid-3-sulfate (LCA-3-S) and dehydroepiandrosterone-3-sulfate are likely not active (EC₅₀>100 uM), whereas their unsulfated parent compounds (i.e., LCA and dehydroepiandrosterone) are active (EC₅₀ of 0.58 uM and 3.33 uM, respectively). These data suggest that sulfation at the 3 position abolishes activity.

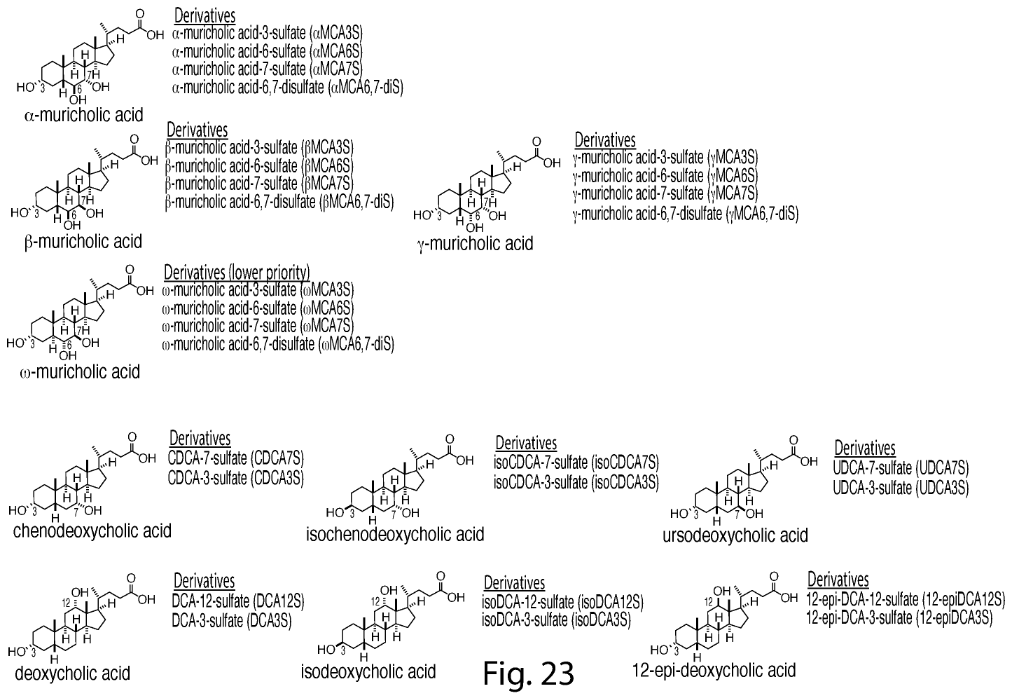

The present data shows that there is a large pocket at C₆-C₇, but not that is hydrophobic. Testing the tolerance of sulfation at both C6 and C7 can involve synthesizing sulfated derivatives of muricholic acids, which are hydroxylated at C6 and C7 (FIG. 23).

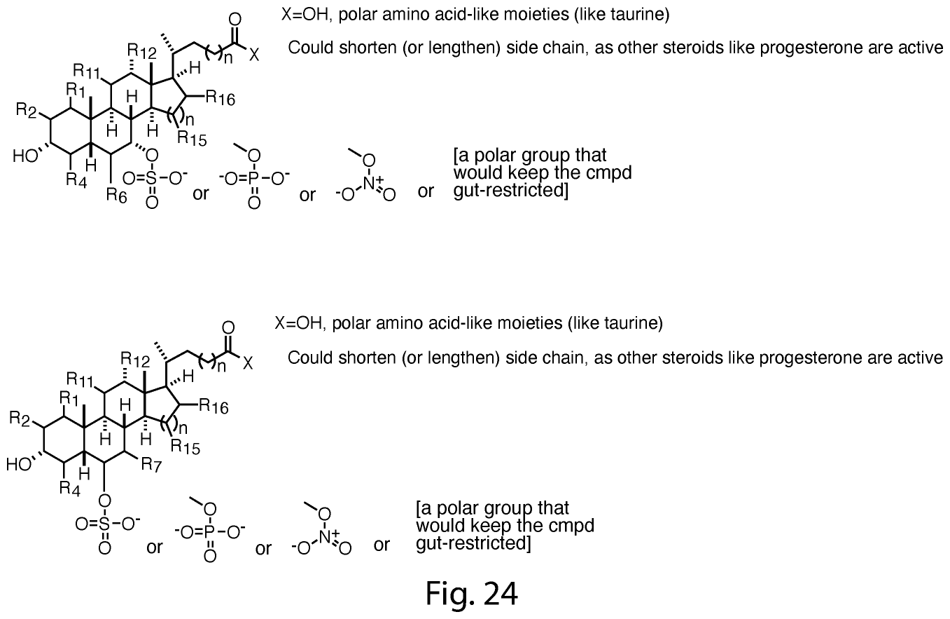

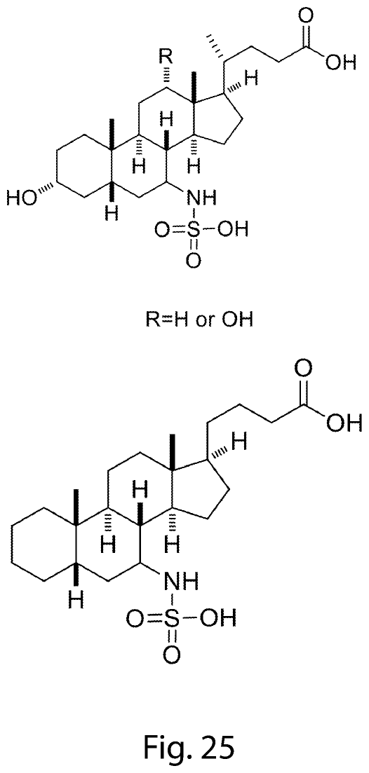

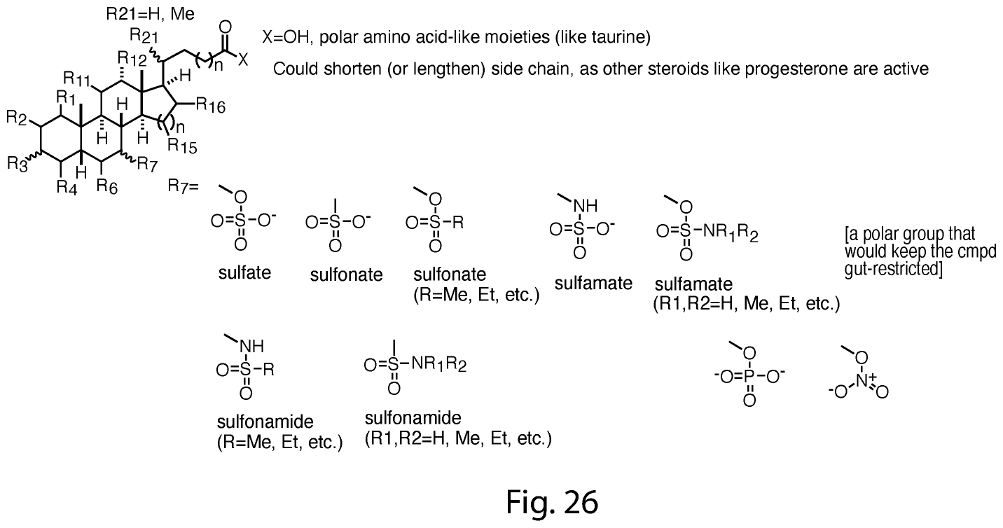

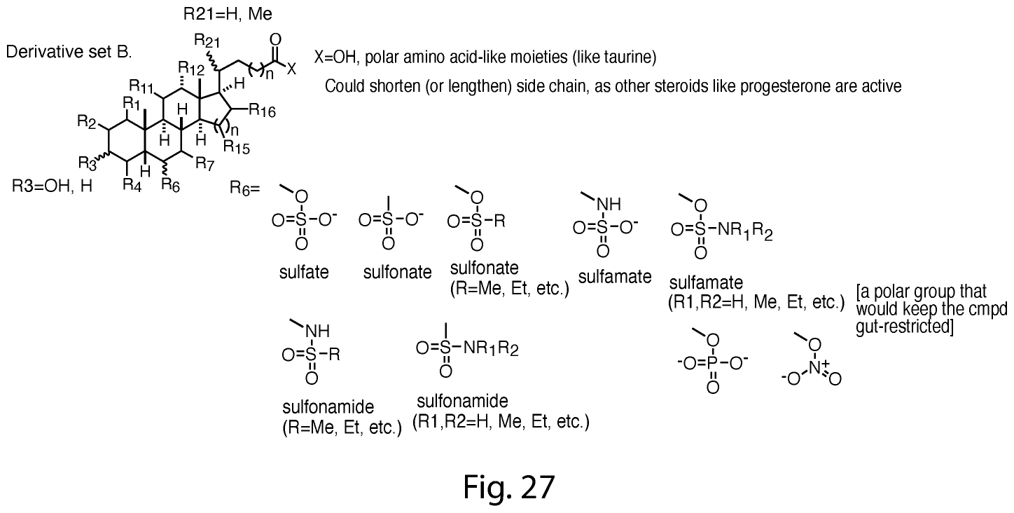

The design and synthesis of milligram quantities synthetic CA7S derivatives were considered (FIG. 24). The compounds in FIG. 24, maintain the potency of or be more potent than CA7S (i.e., lower EC₅₀ values as TGR5 agonists) and remain gut-restricted (i.e., not absorbed into synthetic circulation). To not be bound by a particular theory, it is hypothesized that the sulfate group at C7 (or C6) in addition to any further modifications will maintain activity. Additional examples of cholic acid 7-sulfate derivatives are shown in FIG. 25. Modifications can be made to the R₇ (FIG. 26) and R₆ (FIG. 27) positions of the compound as described herein. A polar group can be added to keep the compounds gut-restricted.

### Example 4: A Microbial Metabolite Remodels the Gut-Liver Axis Following Bariatric Surgery

Obesity and type 2 diabetes have reached epidemic proportions and warrant the need for urgent therapies. Bariatric surgery, in particular sleeve gastrectomy (SG), is currently the most effective and sustainable treatment for obesity, with over 85% patients losing weight and maintaining long-term weight-loss (Abbasi, 2017). Even though weight-loss post-surgery can take six months to a year, patients undergoing SG see immediate resolution in their diabetic phenotypes within hours post-surgery. The molecular mechanisms underlying this immediate and effective amelioration of diabetes remains largely unknown. Research in identifying the underlying mechanisms have documented metabolic changes that occur post-bariatric surgery in human patients and rodent models. Three major changes consistently occur post-surgery: 1. an increase in levels of GLP-1, a circulating incretin hormone; 2. changes in the bile acid (BA) repertoire; and 3. changes to the microbiome. Many studies have documented overall changes to bile acid pools post-surgery. Recently, the importance of studying individual bile acid changes in bariatric surgery has been identified. A specific bile acid, cholic acid 7-sulfate, was identified and is increased post-SG in both mice and human patients. It was determined that CA7S is a potent TGR5 agonist and GLP-1 secretagogue. It was also shown that CA7S induces GLP-1 secretion and blood glucose clearance in diet-induced obese (DIO) mice. However, the mechanism that drives increased production of CA7S post-SG is unknown. Furthermore, there is still no known causal link between SG, the microbiome, and subsequent amelioration of T2D.

Changes to gut microbial community composition following surgery have been shown to influence metabolic outcomes (Medina et al., 2017; Tremaroli et al., 2015). Specifically, SG has been shown to change the relative abundances of gut bacteria, conferring predominance to species that improve diabetic phenotypes and trigger weight-loss (Ryan et al., 2014). In addition, fecal transplants from human patients and mice post-bariatric surgery confer metabolic benefits to obese mice, including improved glucose tolerance, insulin sensitivity, and weight-loss (Liu et al., 2018; Ryan et al., 2014). The molecular link between post-SG changes in the gut microbiome and post-SG metabolic benefits, however, remains largely unclear.

### Results

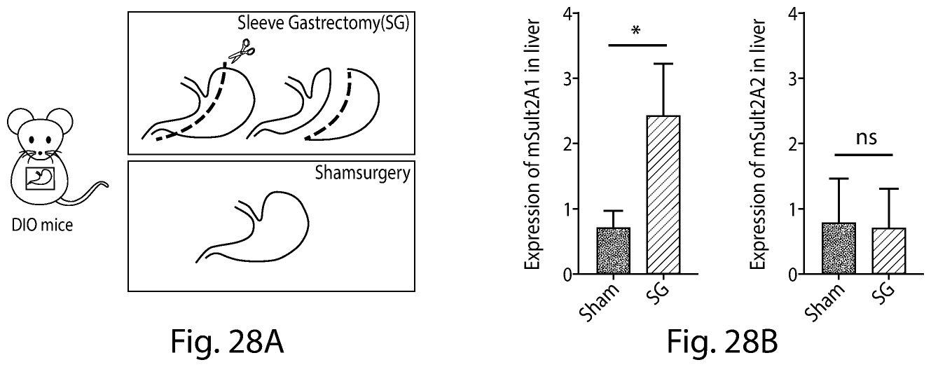

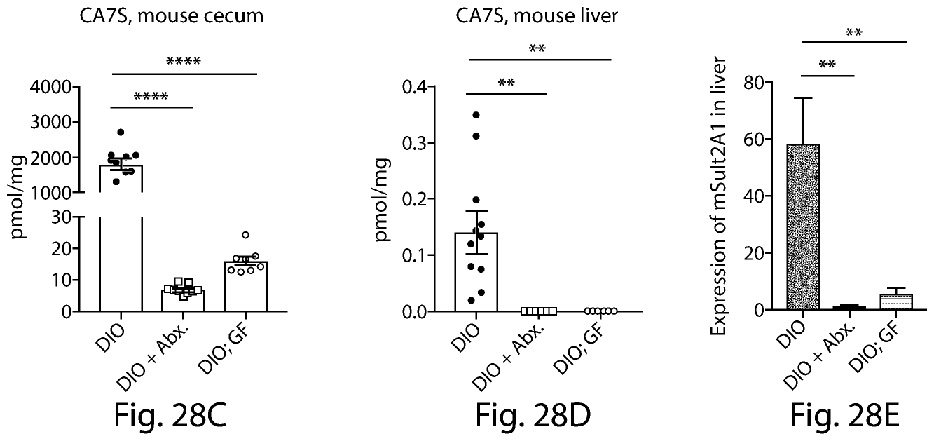

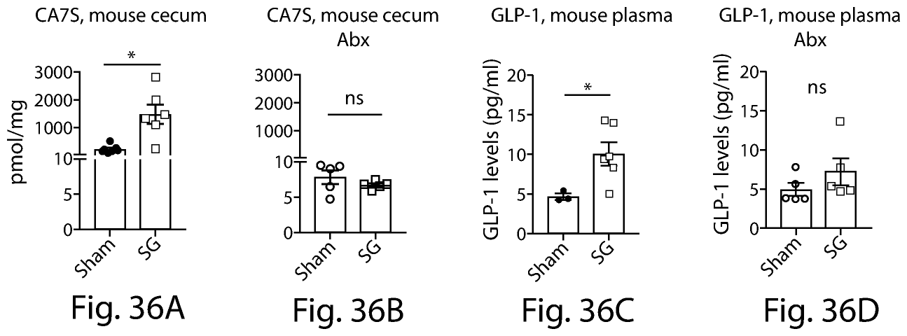

Sleeve gastrectomy (SG) (FIG. 28A) results in beneficial metabolic changes in humans and rodent models, including improved glucose tolerance, insulin sensitivity, and weight loss (Abbasi, 2017). Previous work has found that cholic acid-7-sulfate (CA7S) is a naturally occurring bile acid whose levels are increased in mouse cecal contents and in human feces post-SG (submitted). Sulfation of bile acids primarily occurs in the liver via specific bile acid-sulfotransferase enzymes or SULTs (Alnouti, 2009). Consistently, CA7S was also found to be higher in mouse livers post-SG (submitted). Two isoforms of bile acid-SULTs have been cloned from mice, mSult2a1 and mSult2a2, which specifically and exclusively sulfate bile acids (Alnouti, 2009). It was discovered that mice post-SG have higher expression levels of the mSult2A1 isoform in their livers compared to sham mice, while there was no difference in expression of the mSult2a2 isoform (FIG. 28B). The underlying mechanism that drives increased expression of mSult2A1 was examined. It was discovered that this increased synthesis of CA7S post-SG. A recent study showed that antibiotic-mediated disruption of intestinal microbiota abolished metabolic benefits of SG in mice (Jahansouz et al., 2018). Therefore, it was determined if the microbiome played a role in production of CA7S. For this, CA7S was quantified in diet-induced obese (DIO) mice that were either fully colonized, treated with antibiotics, or germ-free. Remarkably, it was observed that the levels of CA7S in the antibiotic-treated and germ-free mouse intestines were significantly lower by 100 to 150-fold compared to the fully-colonized mice with a functional microbiome (FIG. 28C). CA7S levels were undetectable in livers of antibiotic-treated and germ-free mouse livers. The levels of mSult2a1 expression were significantly reduced in these groups compared to DIO mice (FIG. 28D-E). These results suggest that a microbiome is required for production of CA7S. SG mice displayed higher levels of CA7S in their cecal contents post-surgery compared with shams (FIG. 36A). However, treatment of mice with antibiotics prior to surgery abolished the SG-mediated increase in CA7S levels (FIG. 36B). Notably, the overall levels of CA7S were significantly lower in the antibiotic-treated cohort compared with the untreated mice (FIG. 36A-B). An increase in circulating levels of GLP-1 in DIO mice that were subjected to SG was also found (FIG. 36C). Treatment of DIO mice with antibiotics pre- and post-SG ablated the SG-mediated increase in GLP-1 secretion (FIG. 36D).

These findings begin to determine what microbial factors are inducing the synthesis of CA7S from cholic acid in the liver. Bile acids tightly regulate their own synthesis and sulfation (Alnouti, 2009). These steroidal natural products are synthesized in the liver from cholesterol, stored in the gallbladder, and then released into the intestine upon the ingestion of food. They are then enzymatically modified by resident gut bacteria to produce a class of metabolites called secondary bile acids (Wahlstrom et al., 2016). Primary and secondary bile acids are reabsorbed and pass through the portal vein to the liver (Mertens et al., 2017). The pool of bile acids that reaches the liver then signals through host receptors, in particular, FXR, to control the synthesis of bile acids from cholesterol. Based on the finding that a microbiome is required for production of CA7S and the knowledge that bile acids control their own biosynthesis, it was investigated whether bacterially modified bile acids are causally involved in CA7S production. To not be bound by a particular theory, it was hypothesized that secondary bile acids signal in the liver to induce expression of SULT2A1 and thereby stimulate the synthesis of CA7S.

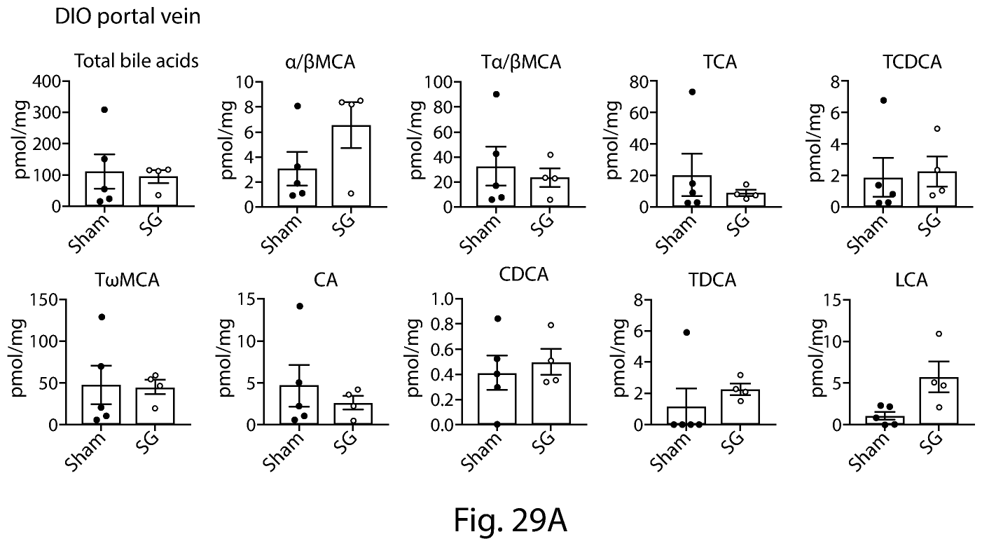

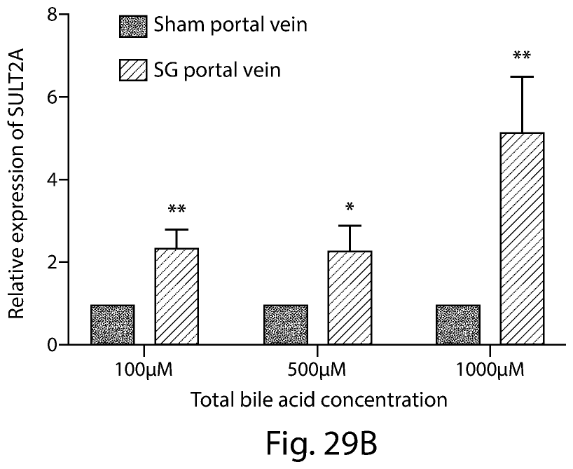

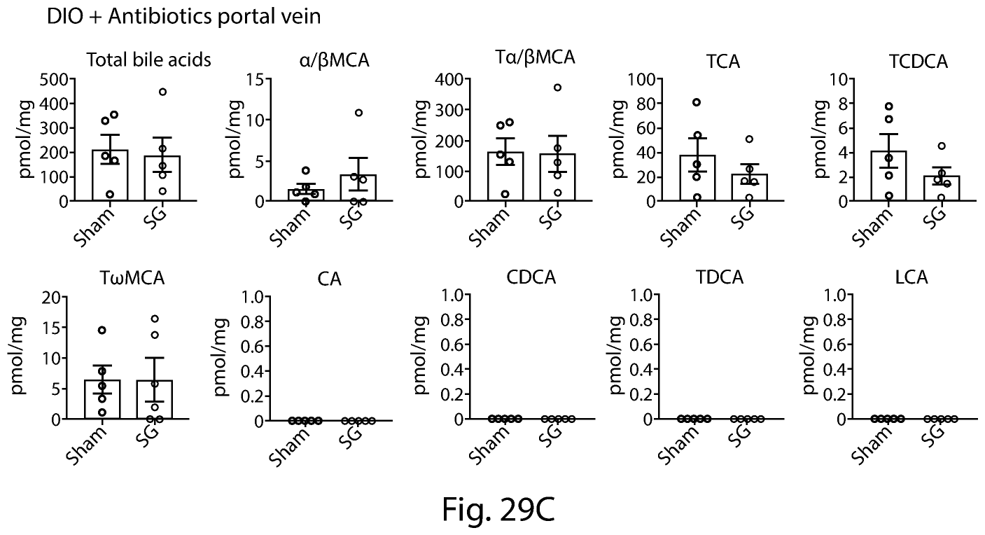

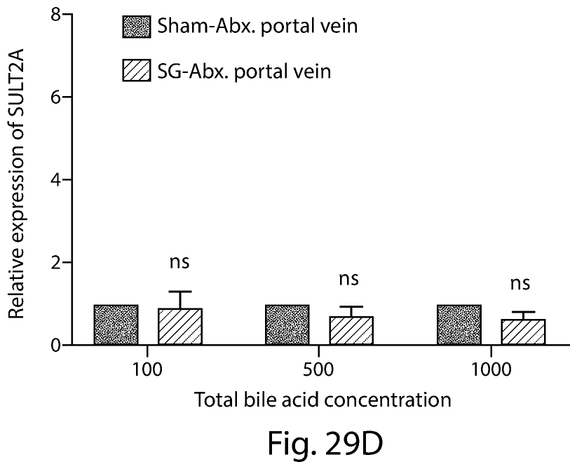

As a first step toward investigating this hypothesis, bile acid profiling was performed on portal veins harvested from sham and SG mice using Ultra-high Performance Liquid Chromatography-Mass Spectrometry (UPLC-MS) (FIG. 29A). The portal vein is the biological conduit by which bacterial metabolites are transported from the gut to the liver and thus acts as a ‘gateway’ allowing cross-talk between the gut microbiome and the liver (Ma et al., 2018). Over 95% of bile acids released into the GI tract are absorbed by the portal vein and recirculated to the liver (Mertens et al., 2017). The liver then extracts 90% of bile acids from portal blood, and these molecules then trigger signaling pathways in hepatocytes by activating canonical receptors (van de Laarschot et al., 2016). The portal vein is the major blood supplier to the liver, providing over 70% of the liver's blood supply. Gut metabolites transported via the portal vein therefore constitute a significant portion of the molecular milieu to which the liver is exposed. Despite the integral role of the liver in the enterohepatic recirculation of bile acids, bile acid profiling of portal vein contents has not yet been reported. To test whether portal vein bile acids can induce expression of SULT, in vitro pools of bile acids were generated that mimic the average physiological concentrations observed in sham and SG portal veins. It was then tested the ability of these reconstituted bile acid pools to induce expression of the human isoform of bile acid-SULT, SULT2A1, in human HepG2 cells (Alnouti, 2009). It was discovered that SG portal vein bile acids significantly induced SULT2A1 expression in vitro in a dose-dependent manner compared to the sham bile acid pools (FIG. 29B).

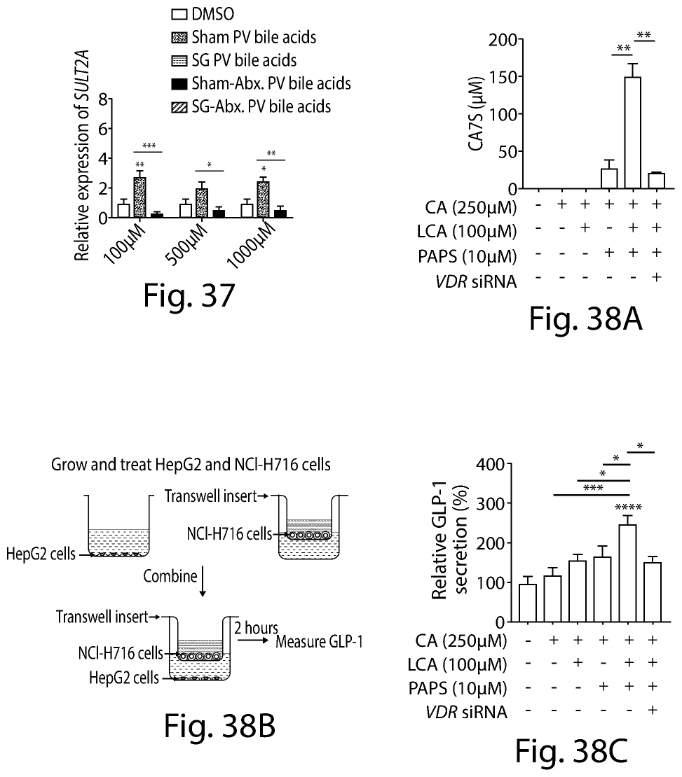

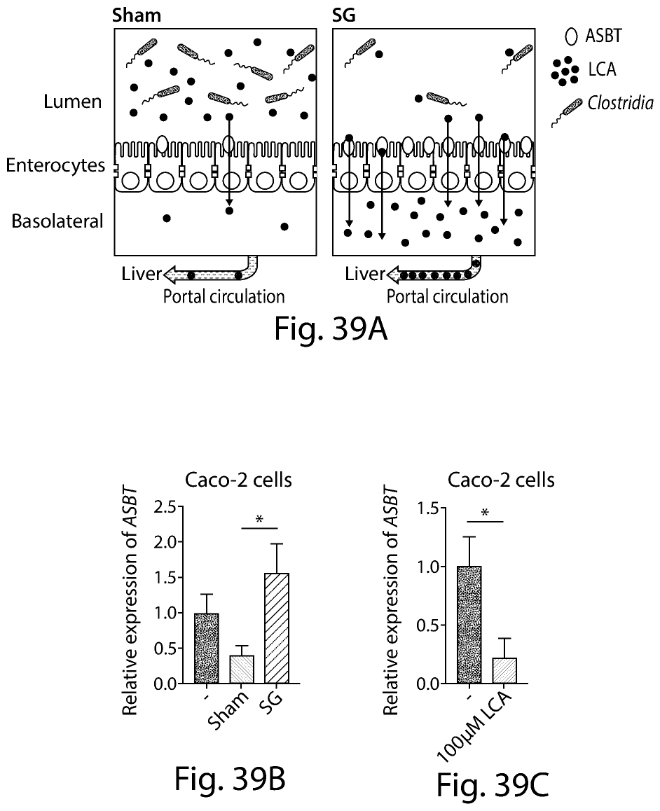

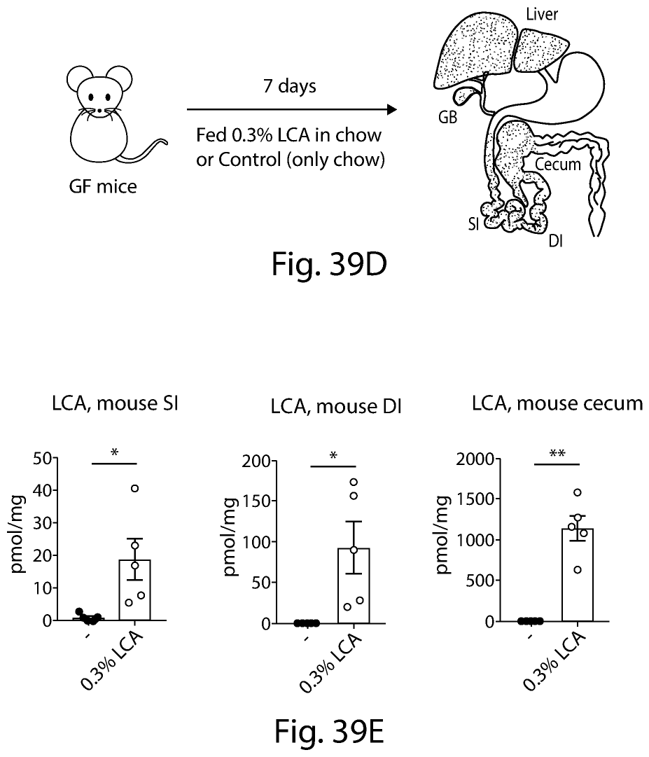

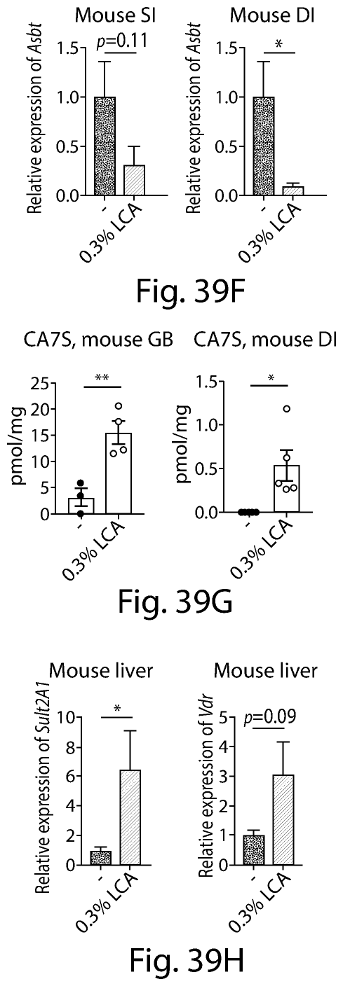

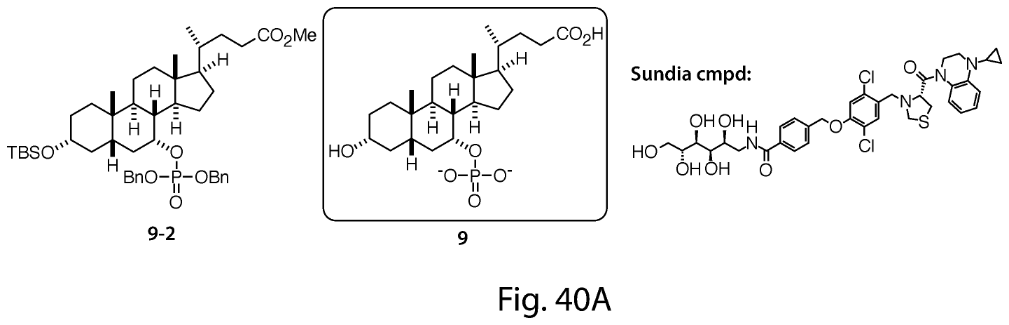

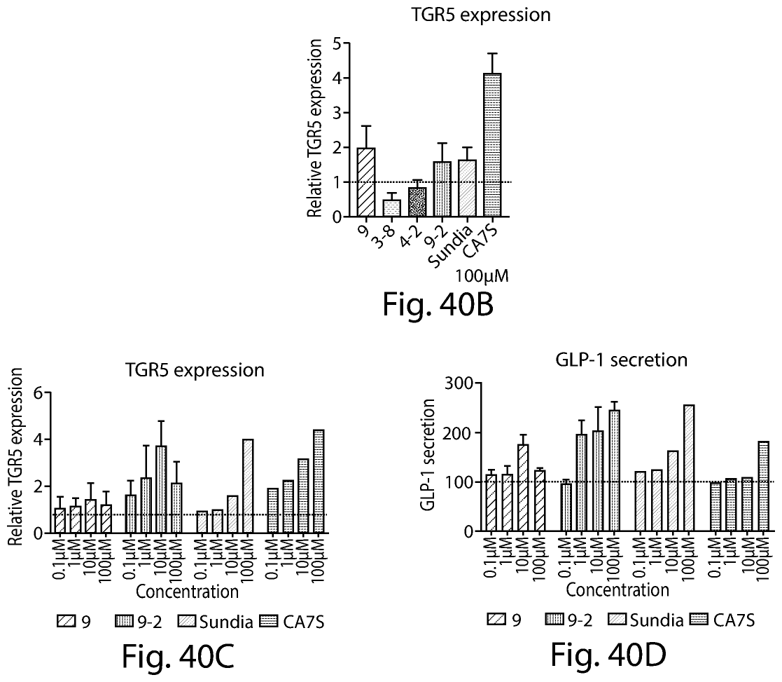

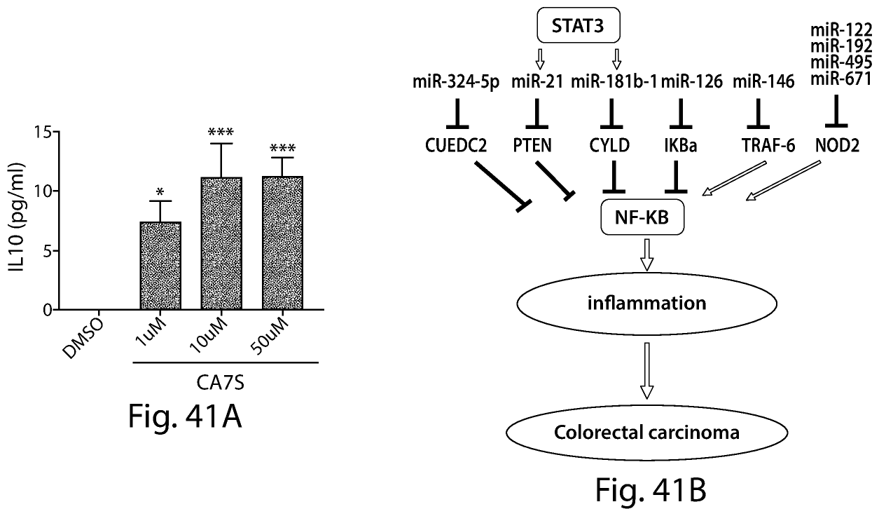

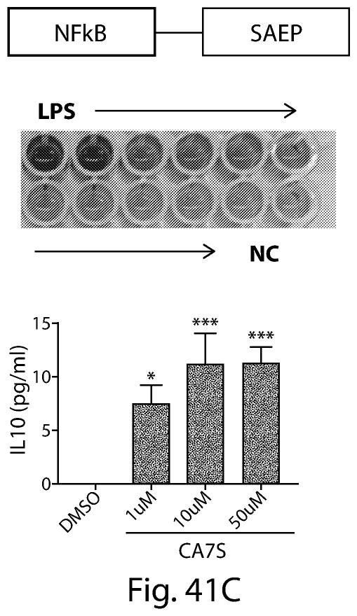

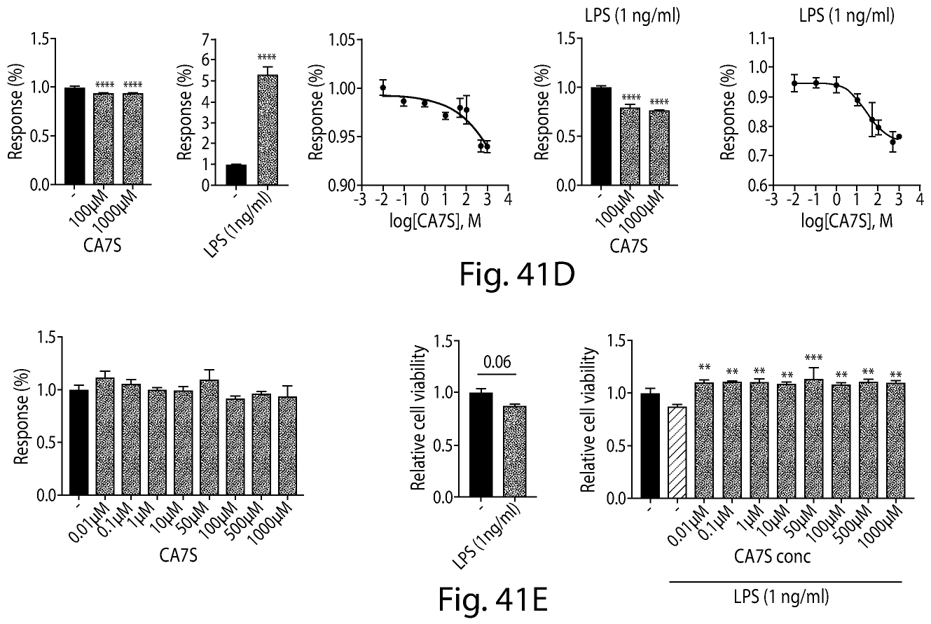

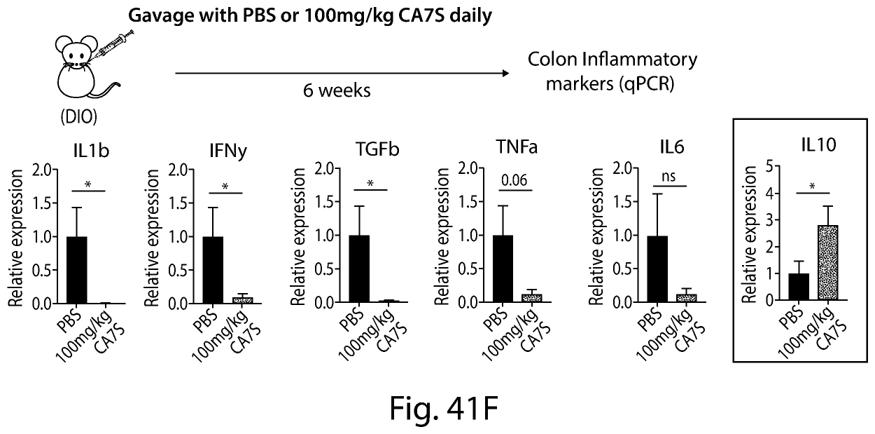

Because antibiotic-treatment abolishes synthesis of CA7S, the bile acids in the portal vein of antibiotic-treated mice subjected to sham surgery or SG were analyzed (FIG. 29C). Strikingly, it was found that bile acid pools from antibiotic-treated SG mice did not induce SULT2A1 expression compared to shams (FIG. 29D and FIG. 37). More importantly, it was found differences in the bile acid repertoire between fully colonized and antibiotic-treated mice. Four bile acids—chenodeoxycholic acid (CDCA), taurodeoxycholic acid (TCDCA), cholic acid (CA), and lithocholic acid (LCA)—were absent in the portal veins of antibiotic-treated mice (FIG. 29C). Treatment of HepG2 cells with these individual bile acids showed that only LCA could induce SULT2A1 expression in a dose dependent manner, while the CDCA, TCDCA, and CA did not induce SULT2A1 expression at all concentrations tested (FIG. 30A). Notably, LCA is a microbiome-derived secondary bile acid, whereas CDCA, TCDCA, and CA are host-produced primary bile acids. This result is consistent with the observation that LCA levels are over 5.5-fold higher in the portal veins of SG mice compared to sham mice, although not statistically significant (p=0.08, Welch's t test; *p=0.03, Student's t test) (FIG. 29A). Moreover, incubation of HepG2 cells with LCA and CA, the precursor of CA7S, resulted in the production of CA7S in vitro (FIG. 30B). Therefore, the present results demonstrate that LCA, a microbial metabolite that is transported from the gut to the liver by the portal vein, affects bile acid synthesis in the liver.

Finally, the receptor(s) that facilitates LCA-mediated induction of SULT expression was identified. Nuclear receptors, including the farnesoid X receptor (FXR), the pregnane X receptor (PXR), the vitamin D receptor (VDR), the constitutive androstane receptor (CAR), the retinoid-related orphan receptors (RORα and RORγ), and the liver X receptor (LXR) have been implicated in their ability to bind LCA and induce expression of SULTs (Fiorucci and Distrutti, 2015; Kakizaki et al., 2009; Runge-Morris et al., 2013). Therefore, a candidate approach was taken and tested these known bile acid receptors for their ability to bind LCA and induce SULT2A1 expression in vitro. It was found that siRNA-mediated knockdown of VDR significantly abolished the LCA-dependent increase in SULT2A1 expression in HepG2 cells, while knockdown of PXR, FXR, CAR, RORα, and RORγ did not significantly affect expression of SULT2A1 (FIG. 30C). It was also observed increased VDR expression in mouse livers post-SG as compared to Sham-operated mouse livers (FIG. 30D). Similar to results obtained with CA7S levels and mSult2A1 expression in antibiotic-treated and germ-free mice, it was observed that VDR expression also requires a microbiome. VDR expression levels were 20-fold lower in germ-free mouse livers and were virtually undetectable in antibiotic-treated animals (FIG. 30E). This study suggests that the observed increase in levels of CA7S following SG is mediated by LCA-induced activation of VDR (FIG. 30F). Therefore, this is the first demonstration of a bacteria-host interaction in bariatric surgery that links a microbial metabolite to amelioration of diabetes.

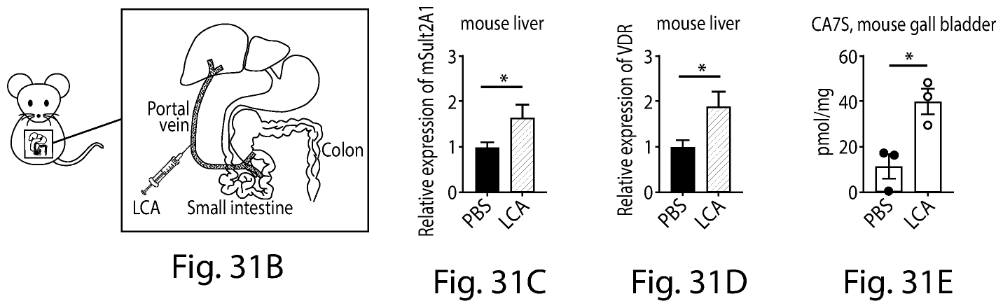

BA analysis of VDR knock-out mouse feces revealed that the levels of CA7S were significantly reduced compared to WT animals, suggesting that VDR is required for production of CA7S in mice (FIG. 31A). To further test whether the proposed LCA-VDR-SULT2A1-CA7S pathway is operable in vivo, LCA (50 μM) was injected directly into the portal vein of DIO mice. This concentration is within an order of magnitude of the physiological concentration of LCA in SG mouse portal veins (FIG. 31B). Two hours after portal vein injection of LCA, a significant increase in expression levels of SULT2A1 and VDR in mouse liver was observed (FIG. 31C,D). Strikingly, an increase in CA7S levels in the gallbladder was also observed, suggesting that LCA injection in the portal vein resulted in CA7S synthesis and subsequent accumulation of this metabolite in the gallbladder (FIG. 31E). Together, these results demonstrate that LCA, a microbial metabolite, is transported from the gut to the liver by the portal vein and induces BA sulfation in the liver. These data also suggest that the observed increase in levels of CA7S following SG is mediated by LCA-induced activation of VDR.

LCA-triggered CA7S synthesis induces GLP-1 secretion in enteroendocrine cell. The synthesis of CA7S in vitro was investigated. Incubation of HepG2 cells with CA, the precursor of CA7S, led to an increase in uptake of CA in hepatocytes, but no detectable CA7S levels were observed (FIG. 38A). The addition of PAPS (30-phosphoadenosine-5′-phosphosulfate), the cofactor required as a sulfonate donor for SULTs, led to production of CA7S in hepatocytes (FIG. 38A). Addition of LCA led to a significant increase in CA7S production (FIG. 38A). Further, siRNA-mediated knockdown of VDR abolished LCA-mediated increase in synthesis of CA7S, demonstrating that LCA requires VDR activation to induce expression of hSULT2A and production of CA7S by hepatocytes (FIG. 38A). The ability of CA7S to be synthesized was investigated using HepG2 cells to induce secretion of GLP-1 by human enteroendocrine L cells (NCI-H716) in a transwell setting (FIG. 38B). Incubation of NCI-H716 cells with HepG2 cells previously induced to synthesize CA7S led to a significant increase in GLP-1 secretion (FIG. 38C). Finally, siRNA-mediated knockdown of VDR, which abolishes synthesis of CA7S, led to a decrease in GLP-1 secretion, suggesting that activation of VDR is necessary for CA7S-mediated induction of GLP-1 secretion (FIG. 38C). These results suggest that the LCA-VDR-SULT pathway functions in the enterohepatic axis to induce production of CA7S and subsequently induce GLP-1 secretion.

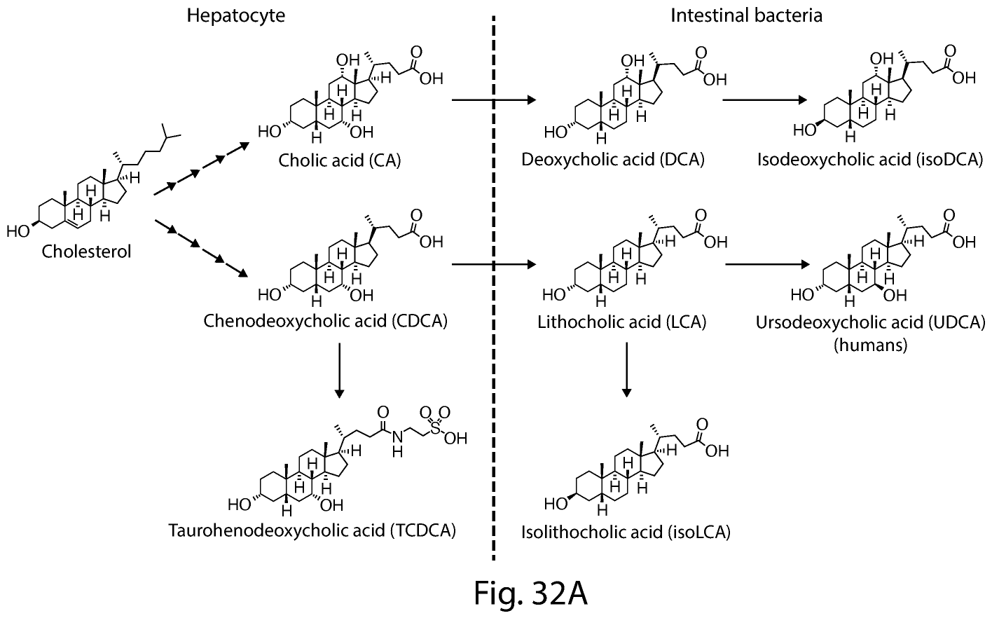

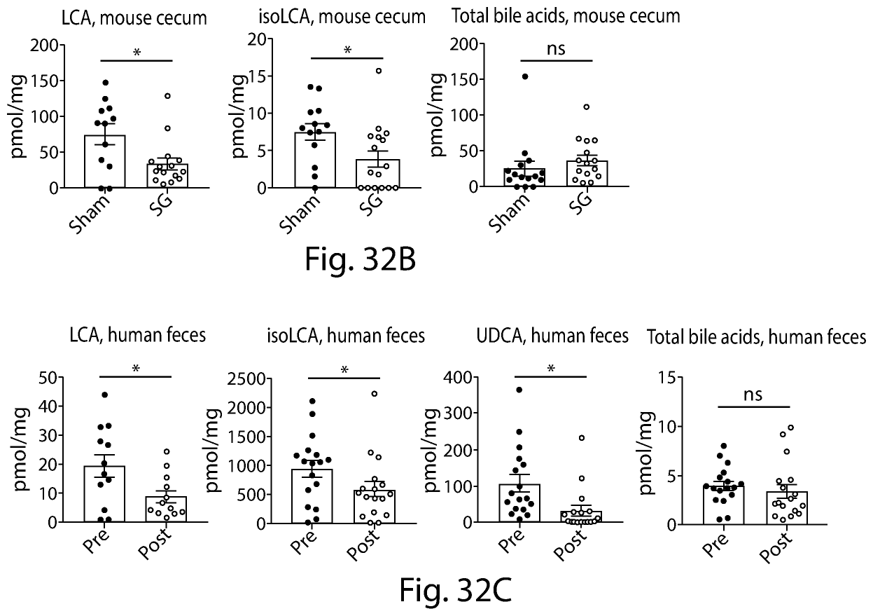

Primary bile acids cholic acid (CA) and chenodeoxycholic acid (CDCA) derived from cholesterol in the liver are modified by specific gut bacteria in the intestine (FIG. 32A). Bacteria metabolize primary bile acids to lithocholic acid (LCA) and deoxycholic acid (DCA) in mice and humans via 7-dehydroxylation reactions (FIG. 31A) (Wahlstrom et al., 2016). LCA and DCA can then be converted via microbial α/β-epimerization reactions to iso-LCA and iso-DCA in humans and mice (FIG. 32A) (Wahlstrom et al., 2016). Interestingly, a significant decrease in LCA and iso-LCA in the gut of mice post-SG was observed, while the levels of DCA and total bile acids were unchanged compared to sham mice (FIG. 32B). Similar changes were observed in humans patients. Levels of the bacterially produced bile acids LCA, iso-LCA, and UDCA were lower in patient feces post-SG, while no change in levels of DCA and total bile acids were observed (FIG. 32C). IsoDCA was not detected in mouse cecum or in human feces post-SG. These results suggest that SG in both mice and humans leads to a reduction in levels of the bacterial metabolite LCA in the colon. These results are consistent with previous studies where bariatric surgery causes a general reduction of Clostridium in the gut, specific strains of which can exclusively and specifically produce LCA, a precursor for synthesis of iso-LCA and UDCA (Magouliotis et al., 2017; Medina et al., 2017).

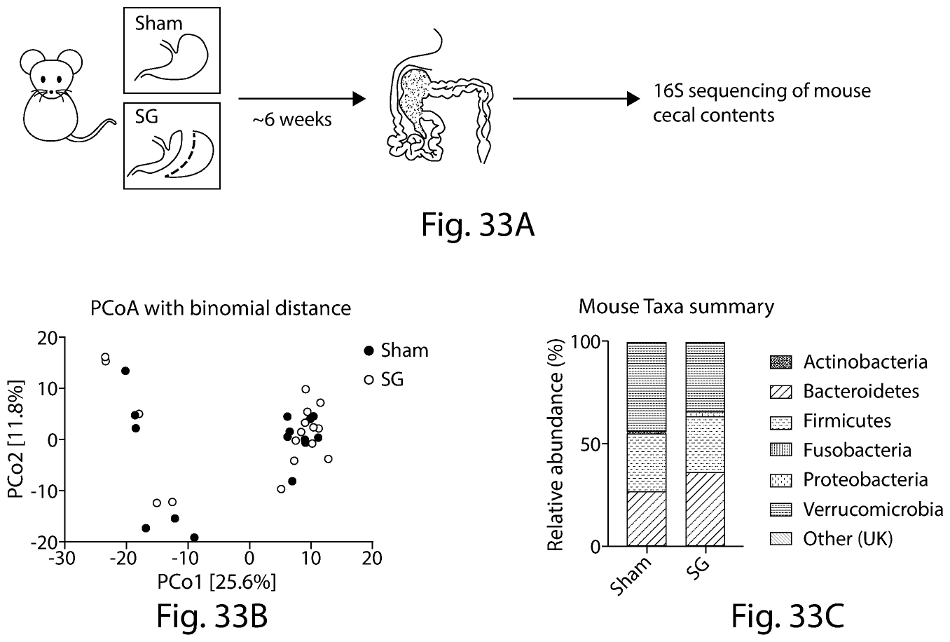

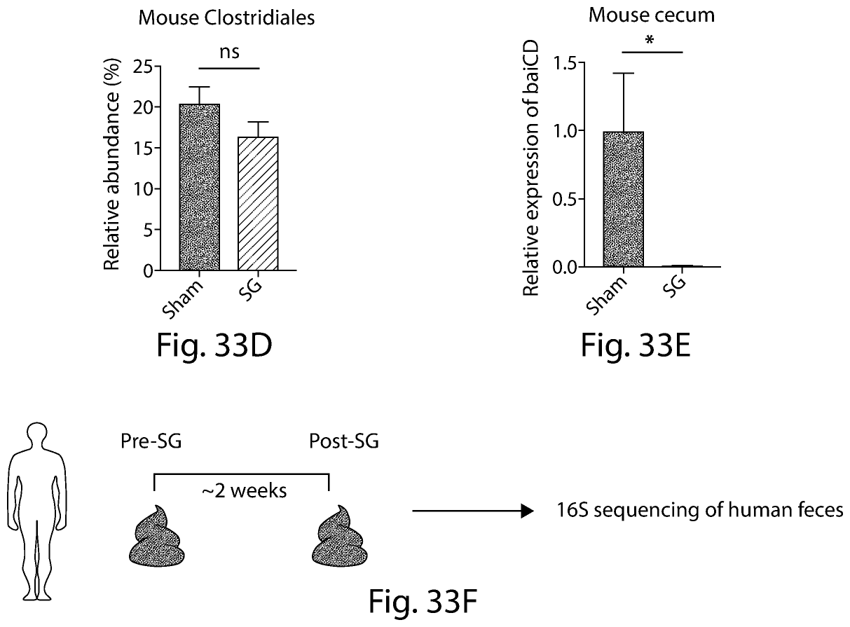

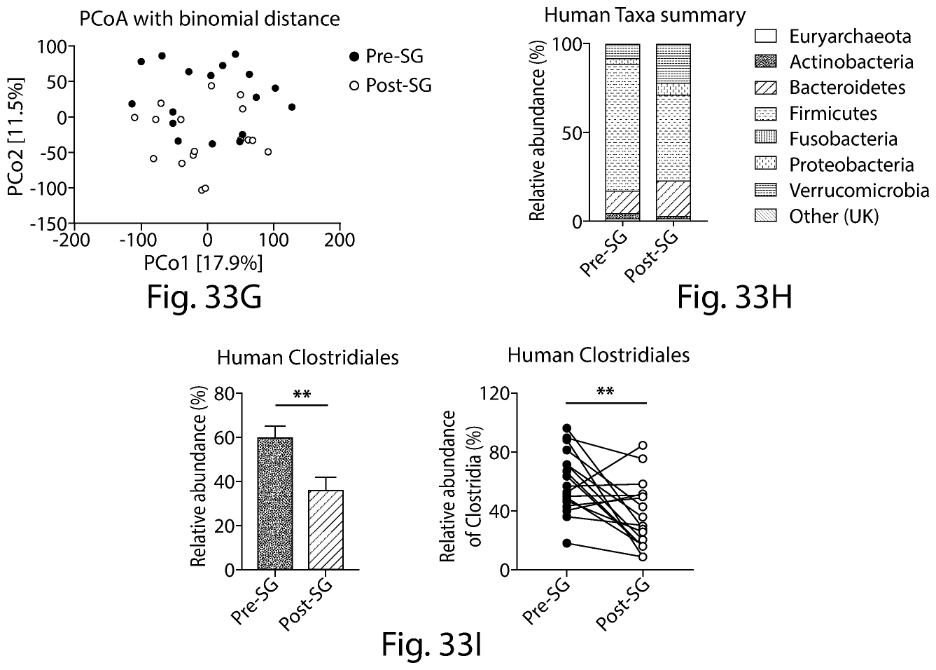

To investigate whether the decrease in gut LCA levels could be a result of a decrease in LCA-producing gut bacteria post-SG, 16S rRNA sequencing was performed on sham and SG mouse cecal contents (FIG. 33A). Consistent with previous studies, it was found that mice post-SG displayed a shift in the microbiome, including an increase in the abundance of Bacteroidetes, Firmicutes, and Proteobacteria phyla that are generally associated with a healthy gut and are reduced in obesity (FIG. 33B,C) (Ryan et al., 2014). The relative abundance of Clostridiales, members of which produce LCA, did not differ between sham and SG cohorts in mice (FIG. 33D). Bacterial synthesis of LCA requires the action of a series of enzymes encoded by genes in the BA inducible (bai) operon (Ridlon et al., 2006) (FIG. 31A). A key enzyme in the LCA biosynthesis cascade is a 3-dehydro-4-BA oxidoreductase encoded by the baiCD gene within the bai operon (Solbach et al., 2018). Real time PCR-based quantification of baiCD mRNA levels in sham and SG mouse cecal contents revealed that mice post-SG exhibited a significant (˜100-fold) decrease in expression of baiCD gene (FIG. 33E). In human patients, the relative abundance of Clostridiales was significantly lower in the post-SG fecal samples (FIG. 33F-I). These results are consistent with previous studies where bariatric surgery resulted in a general reduction of Clostridia in the gut (Magouliotis et al., 2017; McGavigan et al., 2017; Medina et al., 2017). Together, these results suggest that there is a shift in the microbiome following SG that results in a decrease in synthesis of LCA in the gut of mice and humans. Therefore, the observed increase in the microbial metabolite LCA in portal veins of SG mice does not result from an increase in total synthesis of LCA by gut bacteria.

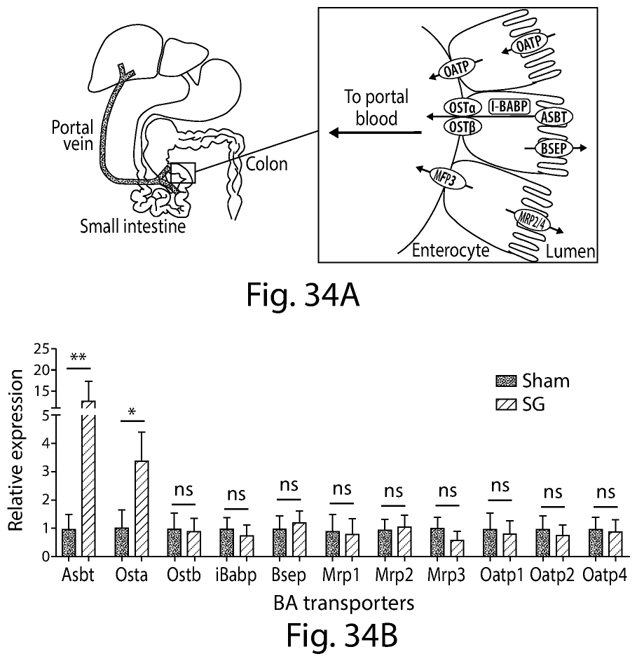

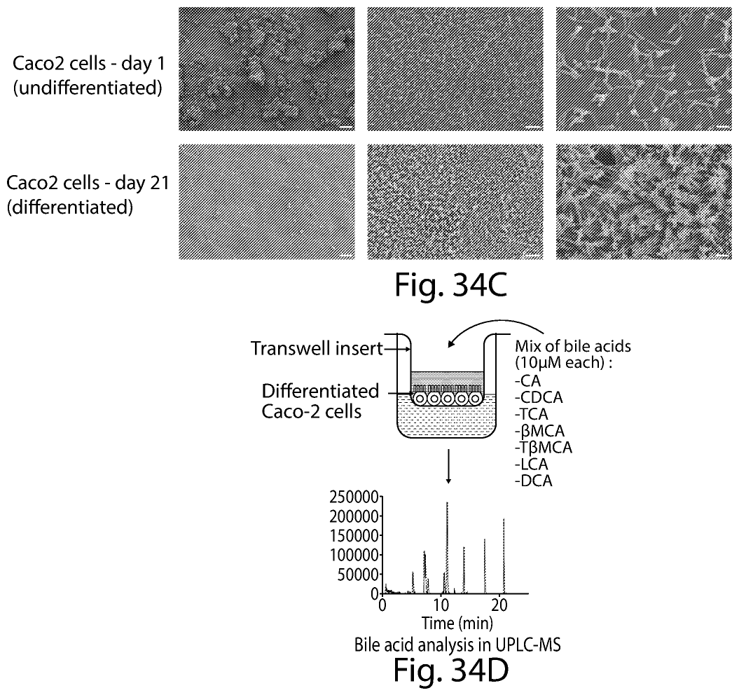

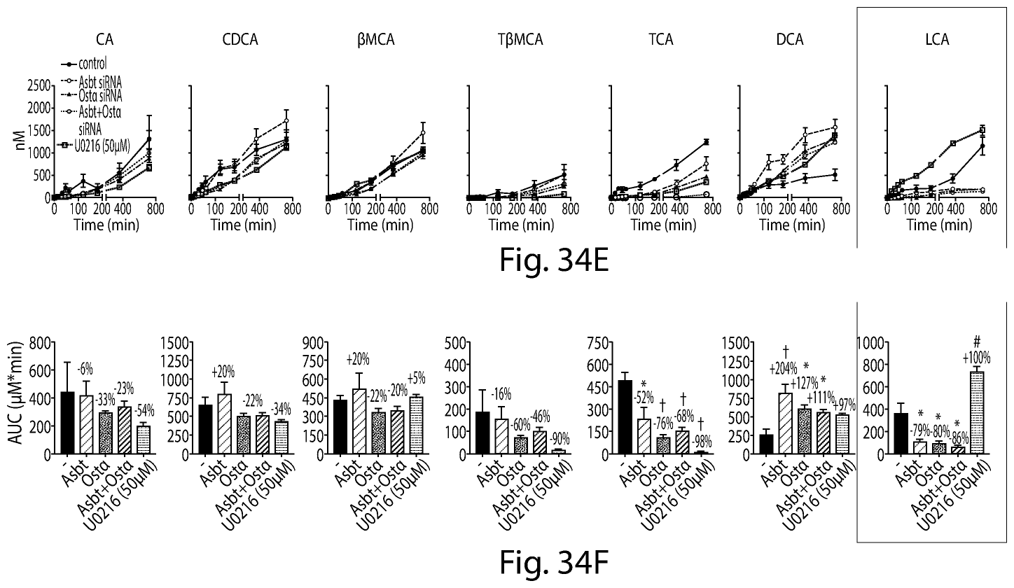

Molecules in the gut can be either be excreted in feces or absorbed from the intestine. Early studies measuring intestinal absorption using gut-infusion experiments found that molecules that are not excreted are either absorbed in the lymphatic system or in the portal vein (McDonald et al., 1980; McDonald and Weidman, 1987). These compounds include fatty acids, hormones, sugars, steroids, and BAs. The molecules absorbed in the lymphatic system enter systemic circulation, while those absorbed in the portal vein drain directly into the liver, where they can induce signaling in hepatocytes and maintain homeostasis (FIG. 34A) (Bemier-Latmani and Petrova, 2017). UPLC-MS-based quantification of BAs in blood samples from sham and SG mice showed no detectable levels of LCA in systemic circulation, suggesting that LCA is not absorbed from the intestine into the lymphatic system. LCA appears to be transported via the portal vein, and the portal vein uptake of this compound appears to be enhanced in SG.

Active absorption of gut metabolites into portal blood or the lymph results in lower levels in the intestine (Dawson et al., 2003; Miyata et al., 2011). Based on the observation that levels of LCA were differentially modulated in distinct compartments of the enterohepatic axis post-SG and that LCA was selectively increased in the portal vein post-SG, the influence of BA transporters in facilitating selective transport of LCA into portal circulation was investigated. Active transport of BAs occurs primarily in the ileum and is mediated by the apical sodium-dependent BA transporter (ASBT) for Na⁺-dependent transport; the organic anion transporting polypeptide (OATP) for Na⁺-independent transport; and members of the ABC family of proteins, including the bile salt export pump (BSEP), the organic solute transporters (OST), and multidrug resistant proteins (MRP) for ATP-dependent transport (Dawson et al., 2009). Further, transport of BAs from the apical to basolateral side of the intestinal epithelium is facilitated by direct binding to the ileal BA-binding protein (I-BABP) (FIG. 34A) (Besnard et al., 2004). Expression levels of these BA transporters in the ileum of sham and SG mice were quantified by qPCR, and it was found that expression of Asbt and Ostα were significantly elevated in the ileum of mice post-SG compared to the sham cohort (FIG. 34B). Asbt and OSTα predominantly mediate transport of BAs from the intestine to the portal vein, and previous studies have observed a similar increase in levels of Asbt post-SG (Ding et al., 2016). Therefore, SG appears to result in increased expression of proteins involved in BA transport into the portal vein.

Substrate specificity of Asbt has been studied for groups of BAs, but not extensively for individual BA molecules (Dawson et al., 2009; Martinez-Augustin and Sanchez de Medina, 2008). Previous research has found that BAs compete for binding Asbt, and that certain amino acid residues of Asbt have different binding affinities to specific BAs (Sun et al., 2006). In particular, Asbt transports dihydroxy BAs such as CDCA and DCA more efficiently than trihydroxy BAs such as TCA and CA (Craddock et al., 1998). However, LCA, a monohydroxy BA, was not studied in this context. Given the observation of increased LCA in the portal vein of SG mice, it was hypothesized that an increase in expression of Asbt and Ostα induces active absorption of LCA from the intestine into portal circulation. Transport of BAs in differentiated human intestinal Caco-2 cells was measured. Caco-2 cells can be differentiated in transwell inserts into a polarized monolayer with intercellular tight junctions and brush border microvilli. This monolayer forms a physical and biochemical barrier to small molecules on the apical side (FIG. 34C) (Ferruzza et al., 2012). This in vitro intestinal model system has been used to study transcytosis of small molecules through the intestinal epithelium (Tan et al., 2018).

In order to test whether LCA is specifically transported through the gut epithelium, a defined mix of predominant gut BAs were added to the apical side of differentiated Caco-2 cells in transwells, and active transport to the basolateral compartment was measured at 12 hours and 24 hours using UPLC-MS (FIG. 34D). Predominant primary BAs found in mice and humans (primary BAs: CA, CDCA, βMCA (beta-muricholic acid); secondary BAs: LCA and DCA; conjugated BAs: TCA (tauro-cholic acid) and TβMCA (tauro-beta-muricholic acid)), were added at a concentration of 10 μM each to the apical side of the transwells (FIG. 34D) (Martinez-Augustin and Sanchez de Medina, 2008). A time course analysis of BA transport over 12 hours was performed to investigate if LCA is transported more efficiently at earlier time points. Similar to our previous results, it was found that LCA does not appear to be transported more efficiently via the epithelial monolayer (FIG. 34E,F). However, siRNA-mediated knockdown of Asbt and/or Ostα specifically abolished transport of LCA and TCA across the epithelial monolayer, suggesting that LCA and TCA require expression of Asbt and Ostα for transcytosis (FIG. 34E,F). Furthermore, overexpression of Asbt by treatment of differentiated Caco-2 cells with the specific MEK inhibitor U0216 resulted in increased transport of LCA through the monolayer (FIG. 34E,F) (Ghosh et al., 2014). Importantly, no other BA exhibited an increase in transport following Asbt overexpression (FIG. 34E,F) (Ghosh et al., 2014). These results suggest that increased expression of Asbt and Ostα in ileum of SG mice results in increased selective transport of LCA into the portal vein.

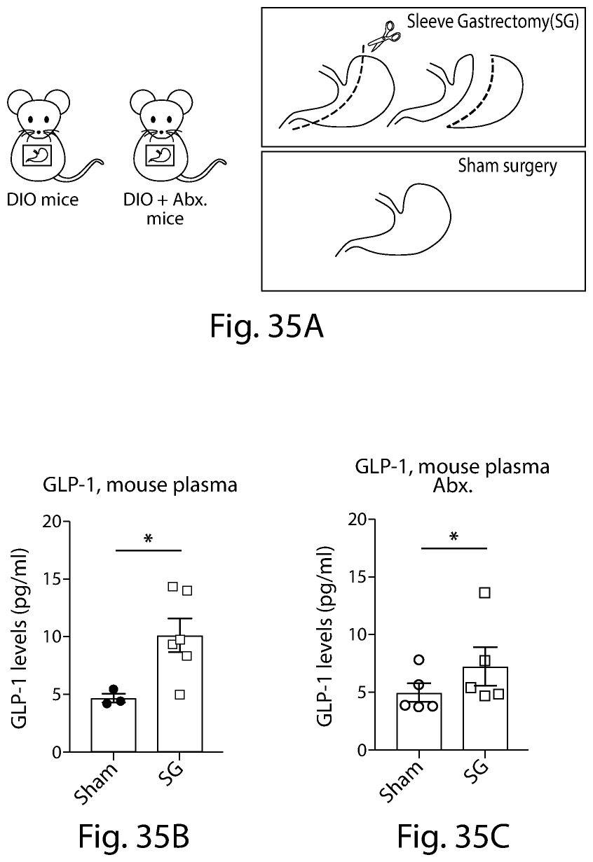

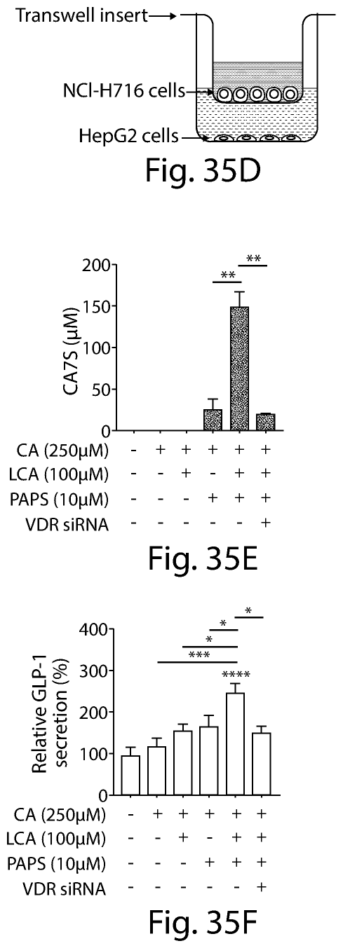

CA7S triggers TGR5 activation and subsequently GLP-1 secretion to improve glucose tolerance in vivo. GLP-1 is a major mediator of diabetes remission post-SG (Kaska et al., 2016; Larraufie et al., 2019). To determine whether the increase in GLP-1 post-SG is dependent on the microbiome, systemic GLP-1 levels were measured in mice subjected to sham and SG that had been treated with antibiotics prior to surgery (FIG. 35A). Consistent with previous studies, an increase in circulating levels of GLP-1 was observed in DIO mice that were subjected to SG (FIG. 35B). However, treatment of DIO mice with antibiotics significantly ablated SG-mediated increase in GLP-1 secretion (FIG. 35C). This result suggests that a microbiome is required to induce SG-mediated GLP-1 secretion in vivo.

LCA-induced increase in expression of SULT2A1 in livers of mice could lead to increased synthesis of CA7S, which in turn could induce GLP-1 secretion. To test this, a co-culture system was utilized in which human liver HepG2 cells were cultured with human enteroendocrine NCI-H716 cells, which secrete GLP-1 (FIG. 35D). The synthesis of CA7S in vitro was investigated. Incubation of HepG2 cells with CA, the precursor of CA7S, led to a time-dependent increase in uptake of CA in hepatocytes, but no detectable CA7S levels were observed (FIG. 35E). Addition of PAPS, the cofactor required as a sulfonate donor for SULTs, led to production of CA7S in hepatocytes (FIG. 35E). Addition of LCA led to a significant increase in CA7S production (FIG. 35E). Further, siRNA-mediated knockdown of VDR abolished LCA-mediated increase in synthesis of CA7S, demonstrating that LCA requires VDR activation to induce expression of SULT2A and production of CA7S by hepatocytes (FIG. 35E).

Next, the ability of CA7S synthesized by HepG2 cells to induce secretion of GLP-1 by NCI-H716 cells grown in a transwell setting was tested. Incubation of NCI-H716 cells with HepG2 cells previously induced to synthesize CA7S was found to lead to a significant increase in GLP-1 secretion (FIG. 35F). Finally, siRNA-mediated knockdown of VDR, which abolishes synthesis of CA7S, lead to a decrease in GLP-1 secretion, suggesting that activation of VDR is important for CA7S-mediated induction of GLP-1 secretion (FIG. 35F). These results suggest that a pathway by which LCA, a metabolite produced exclusively by gut bacteria, can modulate host receptor signaling pathways in both the liver and the GI tract was uncovered (FIG. 30F). This study thus reveals a small molecule-mediated pathway between gut bacteria and host metabolism that likely contributes to amelioration of diabetes post-SG (FIG. 30F).

### SUMMARY

Therapeutic measures for obesity-related diabetes involve invasive surgeries which are expensive and can be life threatening. Therefore, alternative therapeutic interventions that can mimic the outcomes of SG have been researched for decades. Previous work has identified CA7S, a gut-restricted TGR5 agonist that is increased post-SG and is capable of inducing GLP-1 secretion. The therapeutic potential of CA7S has been recently demonstrated in improving hyperglycemia. Studying how bariatric surgeries molecularly and metabolically reprogram the body can allow us to discover drugs that can mimic them. The work provided herein has identified a microbiome-mediated mechanism that drives synthesis of CA7S post-SG. More importantly, it was shown that gut bacteria communicate with the host via the transport of bacterially derived molecules in the portal vein. This could be one of the contributing factors that abolishes benefits of bariatric surgery in animals treated with antibiotics (Jahansouz et al., 2018). Inhibition of bile acid transport in the portal vein has been shown to impair glucose tolerance, insulin sensitivity, and GLP-1 secretion (Shang et al., 2010).

Surprisingly, a decrease in CDCA was observed and its tauro-conjugated form TCDCA in mice post-SG, while in humans a significant decrease in CA levels was found. Previous studies have observed a decrease in either or both primary bile acids in mice and humans, which most likely occurs due to an increase in FXR signaling post-SG (Myronovych et al., 2014; Nemati et al., 2018; Ryan et al., 2014). Further, FXR-activation also increases expression of bile acid transporters ASBT, OSTα and OSTβ, all of which work together to induce absorption of bile acids in to the portal vein (Dawson, 2017). Consistently, bariatric surgery has been shown to increase expression of these transporters in the intestine (Bhutta et al., 2015; Kaska et al., 2016). Moreover, LCA has the greatest affinity for serum albumin which binds bile acids for transport to the liver in the enterohepatic recirculation (Roda et al., 1982). Therefore, without wishing to be bound by a particular theory, it was hypothesized that SG induces an increase in LCA transport in the portal vein, resulting in lower LCA levels in the gut.

The involvement of VDR in mediating LCA-induced synthesis of CA7S is in agreement with recent studies showing the importance of VDR in obesity, type 2 diabetes, and bariatric surgery. Vitamin D deficiency and polymorphisms in the VDR gene have been linked to development of obesity and diabetes, while administration of vitamin D has been shown to improve glucose homeostasis and result in weight-loss (Lespessailles and Toumi, 2017; Manchanda and Bid, 2012; Sisley et al., 2016). More importantly, vitamin D levels positively correlate with diabetes remission post-bariatric surgery (Lespessailles and Toumi, 2017). Furthermore, vitamin D administration has been shown to decrease CDCA levels without affecting CA levels, which could explain lower CDCA and TCDCA levels in mice post-SG in this study (Nishida et al., 2009). Even though vitamin D levels improve diabetic phenotypes, the overall levels of vitamin D does not increase to “healthy” levels post-bariatric surgery (Compher et al., 2008). Therefore, the observations provided herein that shows that LCA, a potent VDR agonist, is increased in portal veins post-SG may compensate for lack of vitamin D, one of the most potent naturally occurring VDR agonist in the liver (Adachi et al., 2005).

### REFERENCES

Abbasi, J. (2017). Unveiling the “Magic” of Diabetes Remission After Weight-Loss Surgery. JAMA 317, 571-574.
Adachi, R., Honma, Y., Masuno, H., Kawana, K., Shimomura, I., Yamada, S., and Makishima, M. (2005). Selective activation of vitamin D receptor by lithocholic acid acetate, a bile acid derivative. J Lipid Res 46, 46-57.
Alnouti, Y. (2009). Bile Acid sulfation: a pathway of bile acid elimination and detoxification. Toxicol Sci 108, 225-246.
Bernier-Latmani, J., and Petrova, T. V. (2017). Intestinal lymphatic vasculature: structure, mechanisms and functions. Nat Rev Gastroenterol Hepatol 14, 510-526.
Besnard, P., Landrier, J. F., Grober, J., and Niot, I. (2004). Is the ileal bile acid-binding protein (I-BABP) gene involved in cholesterol homeostasis?. Med Sci (Paris) 20, 73-77.
Bhutta, H. Y., Rajpal, N., White, W., Freudenberg, J. M., Liu, Y., Way, J., Rajpal, D., Cooper, D. C., Young, A., Tavakkoli, A., et al. (2015). Effect of Roux-en-Y gastric bypass surgery on bile acid metabolism in normal and obese diabetic rats. PLoS One 10, e0122273.
Compher, C. W., Badellino, K. O., and Boullata, J. I. (2008). Vitamin D and the bariatric surgical patient: a review. Obes Surg 18, 220-224.
Craddock, A. L., Love, M. W., Daniel, R. W., Kirby, L. C., Walters, H. C., Wong, M. H., and Dawson, P. A. (1998). Expression and transport properties of the human ileal and renal sodium-dependent bile acid transporter. Am J Physiol 274, G157-169.
Dawson, P. A. (2017). Roles of Ileal ASBT and OSTalpha-OSTbeta in Regulating Bile Acid Signaling. Dig Dis 35, 261-266.
Dawson, P. A., Haywood, J., Craddock, A. L., Wilson, M., Tietjen, M., Kluckman, K., Maeda, N., and Parks, J. S. (2003). Targeted deletion of the ileal bile acid transporter eliminates enterohepatic cycling of bile acids in mice. J Biol Chem 278, 33920-33927.
Dawson, P. A., Lan, T., and Rao, A. (2009). Bile acid transporters. J Lipid Res 50, 2340-2357.
Ding, L., Sousa, K. M., Jin, L., Dong, B., Kim, B. W., Ramirez, R., Xiao, Z., Gu, Y., Yang, Q., Wang, J., et al. (2016). Vertical sleeve gastrectomy activates GPBAR-1/TGR5 to sustain weight loss, improve fatty liver, and remit insulin resistance in mice. Hepatology 64, 760-773.
Ferruzza, S., Rossi, C., Scarino, M. L., and Sambuy, Y. (2012). A protocol for differentiation of human intestinal Caco-2 cells in asymmetric serum-containing medium. Toxicol In Vitro 26, 1252-1255.
Fiorucci, S., and Distrutti, E. (2015). Bile Acid-Activated Receptors, Intestinal Microbiota, and the Treatment of Metabolic Disorders. Trends Mol Med 21, 702-714.
Ghosh, A., Chen, F., Banerjee, S., Xu, M., and Shneider, B. L. (2014). c-Fos mediates repression of the apical sodium-dependent bile acid transporter by fibroblast growth factor-19 in mice. Am J Physiol Gastrointest Liver Physiol 306, G163-171.
Jahansouz, C., Staley, C., Kizy, S., Xu, H., Hertzel, A. V., Coryell, J., Singroy, S., Hamilton, M., DuRand, M., Bernlohr, D. A., et al. (2018). Antibiotic-induced Disruption of Intestinal Microbiota Contributes to Failure of Vertical Sleeve Gastrectomy. Ann Surg.
Kakizaki, S., Takizawa, D., Tojima, H., Yamazaki, Y., and Mori, M. (2009). Xenobiotic-sensing nuclear receptors CAR and PXR as drug targets in cholestatic liver disease. Curr Drug Targets 10, 1156-1163.
Kaska, L., Sledzinski, T., Chomiczewska, A., Dettlaff-Pokora, A., and Swierczynski, J. (2016). Improved glucose metabolism following bariatric surgery is associated with increased circulating bile acid concentrations and remodeling of the gut microbiome. World J Gastroenterol 22, 8698-8719.
Larraufie, P., Roberts, G. P., McGavigan, A. K., Kay, R. G., Li, J., Leiter, A., Melvin, A., Biggs, E. K., Ravn, P., Davy, K., et al. (2019). Important Role of the GLP-1 Axis for Glucose Homeostasis after Bariatric Surgery. Cell Rep 26, 1399-1408 e1396.
Lespessailles, E., and Toumi, H. (2017). Vitamin D alteration associated with obesity and bariatric surgery. Exp Biol Med (Maywood) 242, 1086-1094.
Liu, H., Hu, C., Zhang, X., and Jia, W. (2018). Role of gut microbiota, bile acids and their cross-talk in the effects of bariatric surgery on obesity and type 2 diabetes. J Diabetes Investig 9, 13-20.
Ma, C., Han, M., Heinrich, B., Fu, Q., Zhang, Q., Sandhu, M., Agdashian, D., Terabe, M., Berzofsky, J. A., Fako, V., et al. (2018). Gut microbiome-mediated bile acid metabolism regulates liver cancer via NKT cells. Science 360.
Magouliotis, D. E., Tasiopoulou, V. S., Sioka, E., Chatedaki, C., and Zacharoulis, D. (2017). Impact of Bariatric Surgery on Metabolic and Gut Microbiota Profile: a Systematic Review and Meta-analysis. Obes Surg 27, 1345-1357.
Manchanda, P. K., and Bid, H. K. (2012). Vitamin D receptor and type 2 diabetes mellitus: Growing therapeutic opportunities. Indian J Hum Genet 18, 274-275.
Martinez-Augustin, O., and Sanchez de Medina, F. (2008). Intestinal bile acid physiology and pathophysiology. World J Gastroenterol 14, 5630-5640.
McGavigan, A. K., Garibay, D., Henseler, Z. M., Chen, J., Bettaieb, A., Haj, F. G., Ley, R. E., Chouinard, M. L., and Cummings, B. P. (2017). TGR5 contributes to glucoregulatory improvements after vertical sleeve gastrectomy in mice. Gut 66, 226-234.
McDonald, G. B., Saunders, D. R., Weidman, M., and Fisher, L. (1980). Portal venous transport of long-chain fatty acids absorbed from rat intestine. Am J Physiol 239, G141-150.
McDonald, G. B., and Weidman, M. (1987). Partitioning of polar fatty acids into lymph and portal vein after intestinal absorption in the rat. Q J Exp Physiol 72, 153-159.
Medina, D. A., Pedreros, J. P., Turiel, D., Quezada, N., Pimentel, F., Escalona, A., and Garrido, D. (2017). Distinct patterns in the gut microbiota after surgical or medical therapy in obese patients. PeerJ 5, e3443.
Mertens, K. L., Kalsbeek, A., Soeters, M. R., and Eggink, H. M. (2017). Bile Acid Signaling Pathways from the Enterohepatic Circulation to the Central Nervous System. Front Neurosci 11, 617.
Miyata, M., Yamakawa, H., Hamatsu, M., Kuribayashi, H., Takamatsu, Y., and Yamazoe, Y. (2011). Enterobacteria modulate intestinal bile acid transport and homeostasis through apical sodium-dependent bile acid transporter (SLC10A2) expression. J Pharmacol Exp Ther 336, 188-196.
Myronovych, A., Kirby, M., Ryan, K. K., Zhang, W., Jha, P., Setchell, K. D., Dexheimer, P. J., Aronow, B., Seeley, R. J., and Kohli, R. (2014). Vertical sleeve gastrectomy reduces hepatic steatosis while increasing serum bile acids in a weight-loss-independent manner. Obesity (Silver Spring) 22, 390-400.
Nemati, R., Lu, J., Dokpuang, D., Booth, M., Plank, L. D., and Murphy, R. (2018). Increased Bile Acids and FGF19 After Sleeve Gastrectomy and Roux-en-Y Gastric Bypass Correlate with Improvement in Type 2 Diabetes in a Randomized Trial. Obes Surg.
Nishida, S., Ozeki, J., and Makishima, M. (2009). Modulation of bile acid metabolism by 1alpha-hydroxyvitamin D3 administration in mice. Drug Metab Dispos 37, 2037-2044.
Ridlon, J. M., Kang, D. J., and Hylemon, P. B. (2006). Bile salt biotransformations by human intestinal bacteria. J Lipid Res 47, 241-259.
Roda, A., Cappelleri, G., Aldini, R., Roda, E., and Barbara, L. (1982). Quantitative aspects of the interaction of bile acids with human serum albumin. J Lipid Res 23, 490-495.
Runge-Morris, M., Kocarek, T. A., and Falany, C. N. (2013). Regulation of the cytosolic sulfotransferases by nuclear receptors. Drug Metab Rev 45, 15-33.
Ryan, K. K., Tremaroli, V., Clemmensen, C., Kovatcheva-Datchary, P., Myronovych, A., Karns, R., Wilson-Perez, H. E., Sandoval, D. A., Kohli, R., Backhed, F., et al. (2014). FXR is a molecular target for the effects of vertical sleeve gastrectomy. Nature 509, 183-188.
Shang, Q., Saumoy, M., Holst, J. J., Salen, G., and Xu, G. (2010). Colesevelam improves insulin resistance in a diet-induced obesity (F-DIO) rat model by increasing the release of GLP-1. Am J Physiol Gastrointest Liver Physiol 298, G419-424.
Sisley, S. R., Arble, D. M., Chambers, A. P., Gutierrez-Aguilar, R., He, Y., Xu, Y., Gardner, D., Moore, D. D., Seeley, R. J., and Sandoval, D. A. (2016). Hypothalamic Vitamin D Improves Glucose Homeostasis and Reduces Weight. Diabetes 65, 2732-2741.
Solbach, P., Chhatwal, P., Woltemate, S., Tacconelli, E., Buhl, M., Gerhard, M., Thoeringer, C. K., Vehreschild, M., Jazmati, N., Rupp, J., et al. (2018). BaiCD gene cluster abundance is negatively correlated with Clostridium difficile infection. PLoS One 13, e0196977.
Sun, A. Q., Balasubramaniyan, N., Chen, H., Shahid, M., and Suchy, F. J. (2006). Identification of functionally relevant residues of the rat ileal apical sodium-dependent bile acid cotransporter. J Biol Chem 281, 16410-16418.
Tan, H. Y., Trier, S., Rahbek, U. L., Dufva, M., Kutter, J. P., and Andresen, T. L. (2018). A multi-chamber microfluidic intestinal barrier model using Caco-2 cells for drug transport studies. PLoS One 13, e0197101.
Tremaroli, V., Karlsson, F., Werling, M., Stahlman, M., Kovatcheva-Datchary, P., Olbers, T., Fandriks, L., le Roux, C. W., Nielsen, J., and Backhed, F. (2015). Roux-en-Y Gastric Bypass and Vertical Banded Gastroplasty Induce Long-Term Changes on the Human Gut Microbiome Contributing to Fat Mass Regulation. Cell Metab 22, 228-238.
van de Laarschot, L. F., Jansen, P. L., Schaap, F. G., and Olde Damink, S. W. (2016). The role of bile salts in liver regeneration. Hepatol Int 10, 733-740.
Wahlstrom, A., Sayin, S. I., Marschall, H. U., and Backhed, F. (2016). Intestinal Crosstalk between Bile Acids and Microbiota and Its Impact on Host Metabolism. Cell Metab 24, 41-50.

| SEQUENCES | |
|---|---|
| (TGR5 mRNA transcript-Homo sapiens) | |
| SEQ ID NO: 1 | |
| 1 | ctttccgcct agtgagaggc ggtccgattt ggcccttggg gagtgtccgt cgcgttgatc |
| 61 | tgatggattc acgtacacaa caccacattc tatgagattt tgcaggcaaa agtccacaag |
| 121 | ctcgatatat gggacacctg caccggcatt ggatttggcc ccgcaacatc ttaaaggaag |
| 181 | caggctgtga gccaagggga aggcagagga cagaaatgaa tgtgtttcca ggctttcctg |
| 241 | gtggtttatg gcattctcca aactcctatg caagggctat tcctgaccaa gaagatctaa |
| 301 | agagaacgtc tctgaaatca agtccggatg aagaattaag agaaaaaaag tgaatatggt |
| 361 | ttttgctcac agaatggata acagcaagcc acatttgatt attcctacac ttctggtgcc |
| 421 | cctccaaaac cgcagctgca ctgaaacagc cacacctctg ccaagccaat acctgatgga |
| 481 | attaagtgag gagcacagtt ggatgagcaa ccaaacagac cttcactatg tgctgaaacc |
| 541 | cggggaagtg gccacagcca gcatcttctt tgggattctg tggttgtttt ctatcttcgg |
| 601 | caattccctg gtttgtttgg tcatccatag gagtaggagg actcagtcta ccaccaacta |
| 661 | ctttgtggtc tccatggcat gtgctgacct tctcatcagc gttgccagca cgcctttcgt |
| 721 | cctgctccag ttcaccactg gaaggtggac gctgggtagt gcaacgtgca aggttgtgcg |
| 781 | atattttcaa tatctcactc caggtgtcca gatctacgtt ctcctctcca tctgcataga |
| 841 | ccggttctac accatcgtct atcctctgag cttcaaggtg tccagagaaa aagccaagaa |
| 901 | aatgattgcg gcatcgtgga tctttgatgc aggctttgtg acccctgtgc tctttttcta |
| 961 | tggctccaac tgggacagtc attgtaacta tttcctcccc tcctcttggg aaggcactgc |
| 1021 | ctacactgtc atccacttct tggtgggctt tgtgattcca tctgtcctca taattttatt |
| 1081 | ttaccaaaag gtcataaaat atatttggag aataggcaca gatggccgaa cggtgaggag |
| 1141 | gacaatgaac attgtccctc ggacaaaagt gaaaactatc aagatgttcc tcattttaaa |
| 1201 | tctgttgttt ttgctctcct ggctgccttt tcatgtagct cagctatggc acccccatga |
| 1261 | acaagactat aagaaaagtt cccttgtttt cacagctatc acatggatat cctttagttc |
| 1321 | ttcagcctct aaacctactc tgtattcaat ttataatgcc aattttcgga gagggatgaa |
| 1381 | agagactttt tgcatgtcct ctatgaaatg ttaccgaagc aatgcctata ctatcacaac |
| 1441 | aagttcaagg atggccaaaa aaaactacgt tggcatttca gaaatccctt ccatggccaa |
| 1501 | aactattacc aaagactcga tctatgactc atttgacaga gaagccaagg aaaaaaagct |
| 1561 | tgcttggccc attaactcaa atccaccaaa tacttttgtc taagttctca ttctttcaat |
| 1621 | tgttatgcac cagagattaa aaagctttaa ctataaaaac agaagctatt tacatatttg |
| 1681 | ttttcactca actttccaag ggaaatgttt tattttgtaa aatgcattca tttgtttact |
| 1741 | gta |
| (TGR 5 polypeptide-Homo sapiens) | |
| SEQ ID NO: 2 | |
| 1 | mvfahrmdns kphliiptll vplqnrscte tatplpsqyl melseehswm snqtdlhyvl |
| 61 | kpgevatasi ffgilwlfsi fgnslvclvi hrsrrtqstt nyfvvsmaca dllisvastp |
| 121 | fvllqfttgr wtlgsatckv vryfqyltpg vqiyvllsic idrfytivyp lsfkvsreka |
| 181 | kkmiaaswif dagfvtpvlf fygsnwdshc nyflpssweg taytvihflv gfvipsvlii |
| 241 | lfyqkvikyi wrigtdgrtv rrtmnivprt kvktikmfli lnllfllswl pfhvaqlwhp |
| 301 | heqdykkssl vftaitwisf sssaskptly siynanfrrg mketfcmssm kcyrsnayti |
| 361 | ttssrmakkn yvgiseipsm aktitkdsiy dsfdreakek klawpinsnp pntfv |
| (GLP-1-Homo sapiens) | |
| SEQ ID NO: 3 | |
| His-Ala-Glu-Gly-Thr-Phe-Thr-Ser-Asp-Val-Ser-Ser-Tyr-Leu-Glu- | |
| Gly-Gln-Ala-Ala-Lys-Glu-Phe-Ile-Ala-Trp-Leu-Val-Lys-Gly-Arg | |
| (Vitamin D3 receptor isoform VDRA-Homo sapiens) | |
| SEQ ID NO: 4 | |
| 1 | meamaastsl pdpgdfdrnv pricgvcgdr atgfhfnamt cegckgffrr smkrkalftc |
| 61 | pfngdcritk dnrrhcqacr lkrcvdigmm kefiltdeev qrkremilkr keeealkdsl |
| 121 | rpklseeqqr iiailldahh ktydptysdf cqfrppvrvn dgggshpsrp nsrhtpsfsg |
| 181 | dsssscsdhc itssdmmdss sfsnldlsee dsddpsvtle lsqlsmlphl adlvsysiqk |
| 241 | vigfakmipg frdltsedqi vllkssaiev imlrsnesft mddmswtcgn qdykyrvsdv |
| 301 | tkaghsleli eplikfqvgl kklnlheeeh vllmaicivs pdrpgvqdaa lieaiqdrls |
| 361 | ntlqtyircr hpppgshlly akmiqkladl rslneehskq yrclsfqpec smkltplvle |
| 421 | vfgneis |
| (Vitamin D3 receptor isoform VDRA mRNA transcript-Homo sapiens) | |
| SEQ ID NO: 5 | |
| 1 | ctgcttgtca aaaggcggca gcggagccgt gtgcgccggg agcgcggaac agcttgtcca |
| 61 | cccgccggcc ggaccagaag cctttgggtc tgaagtgtct gtgagacctc acagaagagc |
| 121 | acccctgggc tccacttacc tgccccctgc tccttcaggg atggaggcaa tggcggccag |
| 181 | cacttccctg cctgaccctg gagactttga ccggaacgtg ccccggatct gtggggtgtg |
| 241 | tggagaccga gccactggct ttcacttcaa tgctatgacc tgtgaaggct gcaaaggctt |
| 301 | cttcaggcga agcatgaagc ggaaggcact attcacctgc cccttcaacg gggactgccg |
| 361 | catcaccaag gacaaccgac gccactgcca ggcctgccgg ctcaaacgct gtgtggacat |
| 421 | cggcatgatg aaggagttca ttctgacaga tgaggaagtg cagaggaagc gggagatgat |
| 481 | cctgaagcgg aaggaggagg aggccttgaa ggacagtctg cggcccaagc tgtctgagga |
| 541 | gcagcagcgc atcattgcca tactgctgga cgcccaccat aagacctacg accccaccta |
| 601 | ctccgacttc tgccagttcc ggcctccagt tcgtgtgaat gatggtggag ggagccatcc |
| 661 | ttccaggccc aactccagac acactcccag cttctctggg gactcctcct cctcctgctc |
| 721 | agatcactgt atcacctctt cagacatgat ggactcgtcc agcttctcca atctggatct |
| 781 | gagtgaagaa gattcagatg acccttctgt gaccctagag ctgtcccagc tctccatgct |
| 841 | gccccacctg gctgacctgg tcagttacag catccaaaag gtcattggct ttgctaagat |
| 901 | gataccagga ttcagagacc tcacctctga ggaccagatc gtactgctga agtcaagtgc |
| 961 | cattgaggtc atcatgttgc gctccaatga gtccttcacc atggacgaca tgtcctggac |
| 1021 | ctgtggcaac caagactaca agtaccgcgt cagtgacgtg accaaagccg gacacagcct |
| 1081 | ggagctgatt gagcccctca tcaagttcca ggtgggactg aagaagctga acttgcatga |
| 1141 | ggaggagcat gtcctgctca tggccatctg catcgtctcc ccagatcgtc ctggggtgca |
| 1201 | ggacgccgcg ctgattgagg ccatccagga ccgcctgtcc aacacactgc agacgtacat |
| 1261 | ccgctgccgc cacccgcccc cgggcagcca cctgctctat gccaagatga tccagaagct |
| 1321 | agccgacctg cgcagcctca atgaggagca ctccaagcag taccgctgcc tctccttcca |
| 1381 | gcctgagtgc agcatgaagc taacgcccct tgtgctcgaa gtgtttggca atgagatctc |
| 1441 | ctgactagga cagcctgtgg cggtgcctgg gtggggctgc tcctccaggg ccacgtgcca |
| 1501 | ggcccggggc tggcggctac tcagcagccc tcctcacccc gtctggggtt cagcccctcc |
| 1561 | tctgccacct cccctatcca cccagcccat tctctctcct gtccaaccta acccctttcc |
| 1621 | tgcgggcttt tccccggtcc cttgagacct cagccatgag gagttgctgt ttgtttgaca |
| 1681 | aagaaaccca agtgggggca gagggcagag gctggaggca gggccttgcc cagagatgcc |
| 1741 | tccaccgctg cctaagtggc tgctgactga tgttgaggga acagacagga gaaatgcatc |
| 1801 | cattcctcag ggacagagac acctgcacct ccccccactg caggccccgc ttgtccagcg |
| 1861 | cctagtgggg tctccctctc ctgcctactc acgataaata atcggcccac agctcccacc |
| 1921 | ccaccccctt cagtgcccac caacatccca ttgccctggt tatattctca cgggcagtag |
| 1981 | ctgtggtgag gtgggttttc ttcccatcac tggagcacca ggcacgaacc cacctgctga |
| 2041 | gagacccaag gaggaaaaac agacaaaaac agcctcacag aagaatatga cagctgtccc |
| 2101 | tgtcaccaag ctcacagttc ctcgccctgg gtctaagggg ttggttgagg tggaagccct |
| 2161 | ccttccacgg atccatgtag caggactgaa ttgtccccag tttgcagaaa agcacctgcc |
| 2221 | gacctcgtcc tccccctgcc agtgccttac ctcctgccca ggagagccag ccctccctgt |
| 2281 | cctcctcgga tcaccgagag tagccgagag cctgctcccc caccccctcc ccaggggaga |
| 2341 | gggtctggag aagcagtgag ccgcatcttc tccatctggc agggtgggat ggaggagaag |
| 2401 | aattttcaga ccccagcggc tgagtcatga tctccctgcc gcctcaatgt ggttgcaagg |
| 2461 | ccgctgttca cccacagggc taagagctag cgctgccgca ccccagagtg tgggaaggga |
| 2521 | gagcggggca gtctcgggtg gctagtcaga gagagtgttt gggggttccg tgatgtaggg |
| 2581 | taaggtgcct tcttattctc actccaccac ccaaaagtca aaaggtgcct gtgaggcagg |
| 2641 | ggcggagtga tacaacttca agtgcatgct ctctgcagcc agcccagccc agctggtggg |
| 2701 | aagcgtctgt ccgtttactc caaggtgggg tctttgtgag agtgagctgt aggtgtgcgg |
| 2761 | gaccggtaca gaaaggcgtt cttcgaggtg gatcacagag gcttcttcag atcagtgctt |
| 2821 | gagtttgggg aatgcggccg cattccctga gtcaccagga atgttaaagt cagtgggaac |
| 2881 | gtgactgccc caactcctgg aagctgtgtc cttgcacctg catccgtagt tccctgaaaa |
| 2941 | cccagagagg aatcagactt cacactgcaa gagccttggt gtccacctgg ccccatgtct |
| 3001 | ctcagaattc ttcaggtgga aaaacatctg aaagccacgt tccttactgc agaatagcat |
| 3061 | atatatcgct taatcttaaa tttattagat atgagttgtt ttcagactca gactccattt |
| 3121 | gtattatagt ctaatataca gggtagcagg taccactgat ttggagatat ttatgggggg |
| 3181 | agaacttaca ttgtgaaact tctgtacatt aattattatt gctgttgtta ttttacaagg |
| 3241 | gtctagggag agacccttgt ttgattttag ctgcagaacg tattggtcca gcttgctctt |
| 3301 | cagtgggaga aaacacttgt aagttgctaa acgagtcaat cccctcattc aggaaaactg |
| 3361 | acagaggagg gcgtgactca cccaagcata tataactagc tagaagtggg ccaggacagg |
| 3421 | cccggcgcgg tggctcacgc ctgtaatccc agcagtttgg gaggtcgagg taggtggatc |
| 3481 | acctgaggtc gggagttcga gaccaacctg accaacatgg agaaaccctg tctctattaa |
| 3541 | aaatacaaaa aaaaaaaaaa aaaaaatagc cgggcatggt ggcgcaagcc tgtaatccca |
| 3601 | gctactcagg aggctgaggc agaagaattg aacccaggag gtggaggttg cagtgagctg |
| 3661 | agatcgtgcc gttactctcc aacctggaca acaagagcga aactccgtct tagaagtgga |
| 3721 | ccaggacagg accagatttt ggagtcatgg tccggtgtcc ttttcactac accatgtttg |
| 3781 | agctcagacc cccactctca ttccccaggt ggctgaccca gtccctgggg gaagccctgg |
| 3841 | atttcagaaa gagcaagtct ggatctggga ccctttcctt ccttccctgg cttgtaactc |
| 3901 | caccaaccca tcagaaggag aaggaaggag actcacctct gcctcaatgt gaatcagacc |
| 3961 | ctaccccacc acgatgtggc cctggcctgc tgggctctcc acctcagcct tggataatgc |
| 4021 | tgttgcctca tctataacat gcatttgtct ttgtaatgtc accaccttcc cagctctccc |
| 4081 | tctggccctg ccttcttcgg ggaactcctg gaaatatcag ttactcagcc ctgggcccca |
| 4141 | ccacctaggc cactcctcca aaggaagtct aggagctggg aggaaaagaa aagaggggaa |
| 4201 | aatgagtttt tatggggctg aacggggaga aaaggtcatc atcgattcta ctttagaatg |
| 4261 | agagtgtgaa atagacattt gtaaatgtaa aacttttaag gtatatcatt ataactgaag |
| 4321 | gagaaggtgc cccaaaatgc aagattttcc acaagattcc cagagacagg aaaatcctct |
| 4381 | ggctggctaa ctggaagcat gtaggagaat ccaagcgagg tcaacagaga aggcaggaat |
| 4441 | gtgtggcaga tttagtgaaa gctagagata tggcagcgaa aggatgtaaa cagtgcctgc |
| 4501 | tgaatgattt ccaaagagaa aaaaagtttg ccagaagttt gtcaagtcaa ccaatgtaga |
| 4561 | aagctttgct tatggtaata aaaatggctc atacttatat agcacttact ttgttgcaag |
| 4621 | tactgctgta aataaatgct ttatgcaaac caaaaaaaaa aaaaaaaaa |
| (SULT2A1-mRNA transcript-Homo sapiens) | |
| SEQ ID NO: 6 | |
| 1 | msddflwfeg iafptmgfrs etlrkvrdef virdedviil typksgtnwl aeilclmhsk |
| 61 | gdakwiqsvp iwerspwves eigytalset esprlfsshl piqlfpksff sskakviylm |
| 121 | rnprdvlvsg yffwknmkfi kkpksweeyf ewfcqgtvly gswfdhihgw mpmreeknfl |
| 181 | llsyeelkqd tgrtiekicq flgktlepee lnlilknssf qsmkenkmsn ysllsvdyvv |
| 241 | dkaqllrkgv sgdwknhftv aqaedfdklf qekmadlpre lfpwe |
| (SULT2A1-mRNA transcript-Homo sapiens) | |
| SEQ ID NO: 7 | |
| 1 | agcctccagc ggtggctaca gttgaaaccc tcacaccacg caggaagagg tcatcatcat |
| 61 | gtcggacgat ttcttatggt ttgaaggcat agctttccct actatgggtt tcagatccga |
| 121 | aaccttaaga aaagtacgtg atgagttcgt gataagggat gaagatgtaa taatattgac |
| 181 | ttaccccaaa tcaggaacaa actggttggc tgagattctc tgcctgatgc actccaaggg |
| 241 | ggatgccaag tggatccaat ctgtgcccat ctgggagcga tcaccctggg tagagagtga |
| 301 | gattgggtat acagcactca gtgaaacgga gagtccacgt ttattctcct cccacctccc |
| 361 | catccagtta ttccccaagt ctttcttcag ttccaaggcc aaggtgattt atctcatgag |
| 421 | aaatcccaga gatgttttgg tgtctggtta ttttttctgg aaaaacatga agtttattaa |
| 481 | gaaaccaaag tcatgggaag aatattttga atggttttgt caaggaactg tgctatatgg |
| 541 | gtcatggttt gaccacattc atggctggat gcccatgaga gaggagaaaa acttcctgtt |
| 601 | actgagttat gaggagctga aacaggacac aggaagaacc atagagaaga tctgtcaatt |
| 661 | cctgggaaag acgttagaac ccgaagaact gaacttaatt ctcaagaaca gctcctttca |
| 721 | gagcatgaaa gaaaacaaga tgtccaatta ttccctcctg agtgttgatt atgtagtgga |
| 781 | caaagcacaa cttctgagaa aaggtgtatc tggggactgg aaaaatcact tcacagtggc |
| 841 | ccaagctgaa gactttgata aattgttcca agagaagatg gcagatcttc ctcgagagct |
| 901 | gttcccatgg gaataacgtc caaaacactc tggatcttat atggagaatg acattgattc |
| 961 | tcctgtcctt gtacatgtac ctgactgggg tcattgtgta agacttatta ttttatcctg |
| 1021 | aaaccttaaa tatcaaacct ctgcatctct gatcccttcc ttgttaaaag ttaccagggt |
| 1081 | tggccaggca cggtggttca tgcctgtaat cccagcacta tgggaggccg agacgggcgg |
| 1141 | atcacgaggt caggagactg agaccatcct ggctaacacg gtgaaacccc atctctacta |
| 1201 | aaaatacaaa aaccaaaaaa aattagccag gcgcattggc tcatgtctgt aatcccagca |
| 1261 | ctttgggagg tcgggggggt gggggaggat cacggggtca ggagatcgag accatcctgg |
| 1321 | ccaacatgat gaaaccctat ctctactaaa aatacaaaaa ttagccgggc atggtggtgc |
| 1381 | acgcctatag tcccagctac tcgggaggct gaggtaggag aatcgtttga actcaggagg |
| 1441 | cagaggttgc aatgagccaa gatcgcgcca ctgcactcca gcctgggtga cagagcgaga |
| 1501 | ccgtctcaaa aagaaagaag tgactagggt tcagagaacc agggttcaaa gcccagggat |
| 1561 | gcaaaggttg cagtgagttg agtcatggga tcccagactt ttttaaatgt ttgcaatgtt |
| 1621 | tcccgtttac agaatgctac aagaataatg tacgtactac ctaaaaggat gtctaaatgt |
| 1681 | ttgttaataa aaataagaaa tagctacagt gacagatttt agagcaaaaa ttagtaataa |
| 1741 | aaataagaaa taaaattaca ggagcaatta aggttccatt tcttttcatc acatgtatct |
| 1801 | ccacaattta ttattgctct gatcttaagg agtgtaattt gcaatctaat tttatcctct |
| 1861 | tactctgtgt tgttaaacca tatacaataa acggtgttta tgga |

What is claimed is:

1. A method for treating diabetes, obesity, or an inflammatory disease in a subject in need thereof, the method comprising: administering to a subject in need thereof an agent that increases levels or activity of a sulfotransferase in the subject.

2. A method for treating diabetes, obesity, or an inflammatory disease in a subject, the method comprising: administering to a subject in need thereof an agent that increases levels or activity of lithocholic acid (LCA) in the subject.

3. A method for treating diabetes, obesity, or an inflammatory disease in a subject, the method comprising: administering to a subject in need thereof an agent that increases levels or activity of vitamin D receptor in the subject.

4. Use of an agent for treating diabetes, obesity, or an inflammatory disease in a subject in need thereof, wherein the agent increases the levels or activity of a sulfotransferase in the subject.

5. Use of an agent for treating diabetes, obesity, or an inflammatory disease in a subject in need thereof, wherein the agent increases the levels or activity of lithocholic acid (LCA) in the subject.

6. Use of an agent for treating diabetes, obesity, or an inflammatory disease in a subject in need thereof, wherein the agent increases the levels or activity of vitamin D receptor in the subject.

7. Use of an agent in a method of manufacture a medicament for treating diabetes, obesity, or an inflammatory disease in a subject in need thereof, wherein the agent increases the levels or activity of a sulfotransferase in the subject.

8. Use of an agent in a method of manufacture a medicament for treating diabetes, obesity, or an inflammatory disease in a subject in need thereof, wherein the agent increases the levels or activity of lithocholic acid (LCA) in the subject.

9. Use of an agent in a method of manufacture a medicament for treating diabetes, obesity, or an inflammatory disease in a subject in need thereof, wherein the agent increases the levels or activity of vitamin D receptor in the subject.

10. The method or use of any one of claims 1 to 9, wherein the agent is a vitamin-D receptor (VDR) agonist.

11. The method or use of claim 10, wherein the VDR agonist induces GLP-1 secretion from a target cell.

12. The method or use of claim 10 or 11, wherein the VDR agonist induces metabolism of cholic acid to cholic acid 7-sulfate from a target cell.

13. The method or use of any one of claim 1 to 9, wherein the agent is selected from the group consisting of a small molecule, an antibody, a peptide, a genome editing system, an antisense oligonucleotide, shRNA, and an siRNA.

14. The method or use of any one of claims 1 to 9, or 13, wherein the agent is a small molecule.

15. The method of use of claim 14, wherein the small molecule is a bile acid.

16. The method or use of any one of claim 1 to 15, wherein the agent is lithocholic acid (LCA), or a derivative of LCA, or a pharmaceutically acceptable salt thereof.

17. The method or use of any one of claim 1 to 16, wherein the agent is lithocholic acid (LCA), or a pharmaceutically acceptable salt thereof.

18. The method or use of any one of claim 1 to 17, wherein the agent is lithocholic acid.

19. The method or use of any one of claims 1 to 18, wherein the agent is formulated with a pharmaceutical composition.

20. The method or use of claim 19, wherein the pharmaceutical composition is formulated to restrict delivery of an agent to the gastrointestinal tract of the subject.

21. The method or use of any one of claims 1 to 20, wherein the diabetes is type I, type II, neonatal, or maturity onset diabetes in the young.

22. The method or use of any one of claims 1 to 20, wherein the inflammatory disease is selected from the group consisting of: Crohn's disease, inflammatory bowel disease, ulcerative colitis, pancreatitis, hepatitis, appendicitis, gastritis, diverticulitis, celiac disease, food intolerance, enteritis, ulcer, and gastroesophageal reflux disease (GERD), psoriatic arthritis, psoriasis, and rheumatoid arthritis.

23. The method or use of claim 1 to 22, wherein the administering reduces glucose levels in the serum of a subject.

24. The method or use of any one of claims 1 to 23, wherein the subject is a mammal.

25. The method or use of any one of claims 1 to 24, wherein the mammal is a human.

26. The method or use of any one of claims 1 to 25, wherein the target cell is a hepatocyte, enteroendocrine cell, an epithelial cell, an L-cell, or a neuron.

27. A method of increasing sulfotransferase levels in a cell, the method comprising: increasing levels or activity of VDR in said cell.

28. The method of claim 27, wherein said increasing levels or activity of VDR comprises administering an agonist of VDR.

29. The method of claim 27 or 28, wherein said increasing levels or activity of VDR comprises administering LCA or derivative of LCA, or a pharmaceutically acceptable salt thereof to the cell.

30. The method of any one of claims 27 to 29, wherein said increasing levels or activity of VDR comprises administering LCA, or a pharmaceutically acceptable salt thereof to the cell.

31. The method of claim 27 or 28, wherein said increasing levels or activity of VDR comprises administering a nucleic acid encoding VDR to the cell.

32. The method of any one of claims 27 to 31, wherein said increasing levels or activity of VDR is in vivo.

33. The method of any one of claims 27 to 32, wherein said increasing levels or activity of VDR is in a mammal.

34. The method of any one of claims 27 to 33, wherein said increasing levels or activity of VDR is in a human.

35. The method of any one of claims 27 to 34, wherein said increasing levels or activity of VDR is in a subject in need of treatment for diabetes, obesity, or an inflammatory disease.

36. The method of any one of claims 27 to 35, wherein the diabetes is type I, type II, neonatal, or maturity onset diabetes in the young.

37. The method of any one of claims 27 to 35, wherein the inflammatory disease is selected from the group consisting of: Crohn's disease, inflammatory bowel disease, ulcerative colitis, pancreatitis, hepatitis, appendicitis, gastritis, diverticulitis, celiac disease, food intolerance, enteritis, ulcer, and gastroesophageal reflux disease (GERD), psoriatic arthritis, psoriasis, and rheumatoid arthritis.

38. The method of any one of claims 27, 28, or 31 to 37, wherein said increasing levels or activity of TGR5 comprises administering a nucleic acid encoding TGR5 to the cell.

39. The method of any one of claims 27, 28, or 31 to 37, wherein said increasing levels or activity of sulfotransferase comprises administering a nucleic acid encoding a sulfotransferase to the cell.

40. The method of any one of claims 27 to 39, wherein the activity of VDR is increased by at least 50%, at least 60%, at least 70%, at least 80%, at least 90%, or more as compared to an appropriate control.

41. The method of any preceding claim, wherein the activity of sulfotransferase is increased by at least 50%, at least 60%, at least 70%, at least 80%, at least 90%, or more as compared to an appropriate control.

42. The method of any preceding claim, wherein the sulfotransferase is SULT2A.

43. The method of any preceding claim, wherein the sulfotransferase is SULT2A1.

44. A compound of Formula (I′): 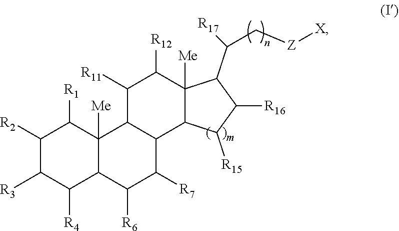
wherein:
n is 1, 2, 3, 4, 5, 6, 7, 8, 9 or 10;
m is 1, 2, 3 or 4;
Z is —C(O)—, —C(O)O—, —C(O)NR₁₈— or —CH₂—;
X is H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, —NHC(O)NHNH₂, or a polar amino acid;
each R₁, R₂, R₄, R₁₁, R₁₂, R₁₅, R₁₆ and R₁₇ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂;
R₃ is —OR₁₉;
each R₆, R₇ and R₁₂ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OR₁₈, —N(R₁₈)₂, —SR₁₈, halogen, —CN, —CHO, —CO₂H, —CO₂R₁₈, —NO₂, —ONO₂, —SO₂Cl, —SO₃⁻, —OSO₃⁻, —NR₁₈SO₃⁻, —PO₃²⁻, —OPO₃²⁻, —OSO₂R₁₈, —SO₂N(R₁₈)₂, —OSO₂N(R₁₈)₂, —NR₁₈SO₂R₁₈, —SO₂N(R₁₈)₂, —NHNH₂, —ONH₂, or —NHC(O)NHNH₂, provided that at least one of R₃, R₆, R₇ and R₁₂ is a polar group;
each R₁₈ is independently H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an oxygen protecting group, a nitrogen protecting group, or a sulfur protecting group;
R₁₉ is an oxygen protecting group;
or a pharmaceutically acceptable salt thereof.

45. The compound of claim 44, wherein the compound is of Formula (II′): 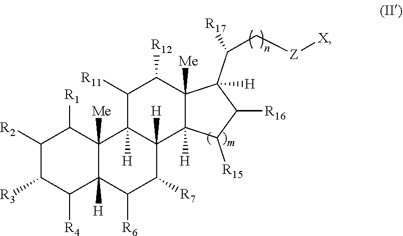
or a pharmaceutically acceptable salt thereof.

46. The compound of claim 44, wherein the compound is of Formula (III′): 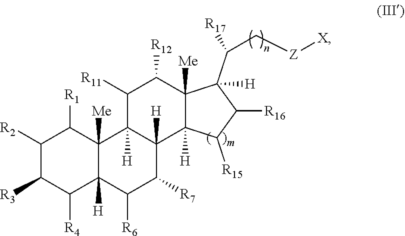
or a pharmaceutically acceptable salt thereof.

47. The compound of claim 44, wherein the compound is of Formula (IV′): 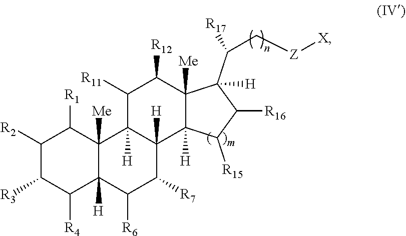
or a pharmaceutically acceptable salt thereof.

48. The compound of claim 44, wherein the compound is of Formula (V′): 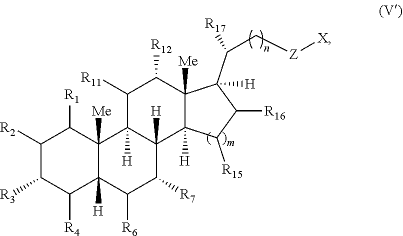
or a pharmaceutically acceptable salt thereof.

49. The compound of claim 44, wherein the compound is of Formula (VI′): 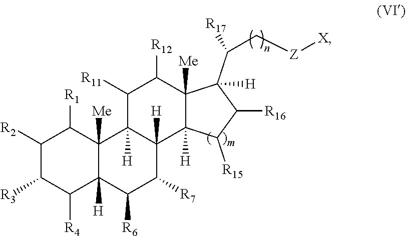
or a pharmaceutically acceptable salt thereof.

50. The compound of claim 44, wherein the compound is of Formula (VII′): 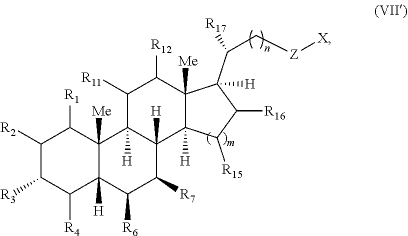
or a pharmaceutically acceptable salt thereof.

51. The compound of claim 44, wherein the compound is of Formula (VIII′): 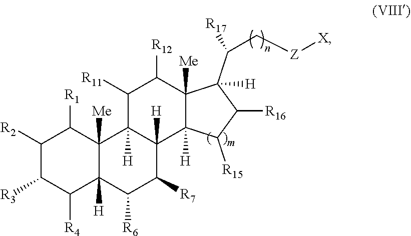
or a pharmaceutically acceptable salt thereof.

52. The compound of claim 44, wherein the compound is of Formula (IX′): 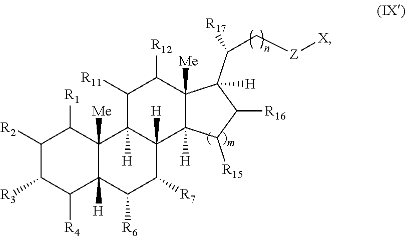
or a pharmaceutically acceptable salt thereof.

53. The compound of claim 44, wherein the compound is of Formula (X′): 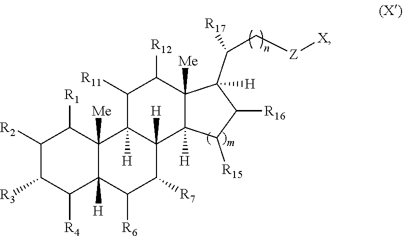
or a pharmaceutically acceptable salt thereof.

54. The compound of claim 44, wherein the compound is of Formula (XI′): 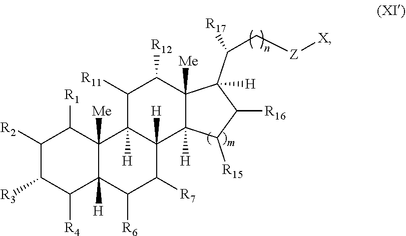
or a pharmaceutically acceptable salt thereof.

55. The compound of claim 44, wherein the compound is of Formula (XII′): 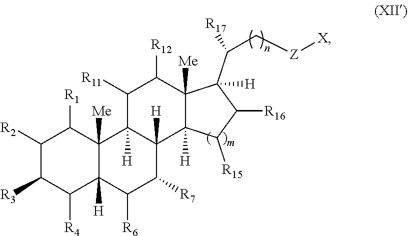
or a pharmaceutically acceptable salt thereof.

56. The compound of claim 44, wherein the compound is of Formula (XIII′): 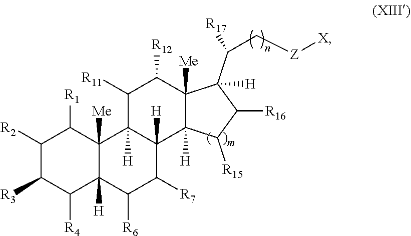
or a pharmaceutically acceptable salt thereof.

57. The compound of claim 44, wherein the compound is of Formula (XIV′): 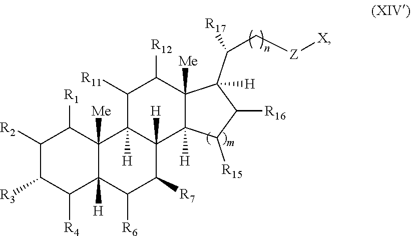
or a pharmaceutically acceptable salt thereof.

58. The compound of claim 44, wherein the compound is of Formula (XV′): 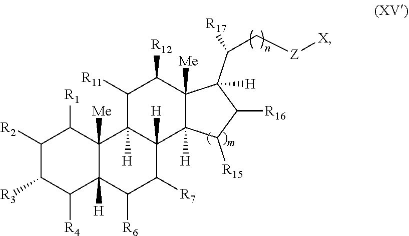
or a pharmaceutically acceptable salt thereof.

59. The compound of any one of claims 44-58, wherein R₁, R₂, R₄, R₁₅ and R₁₆ are H.

60. The compound of any one of claims 44-59, wherein R₁₇ is C₁-C₆ alkyl.

61. The compound of any one of claims 44-60, wherein R₁₇ is methyl.

62. The compound of any one of claims 44-61, wherein R₁₇ is unsubstituted methyl.

63. The compound of any one of claims 44-62, wherein n is 2.

64. The compound of any one of claim 44-63, wherein m is 1.

65. The compound of any one of claims 44-64, wherein at least one of R₆, R₇ and R₁₂ is —OSO₃⁻, —NR₁₈SO₃⁻, or —OPO₃²⁻.

66. The compound of any one claims 44-65, wherein at least one of R₆, R₇ and R₁₂ is —OSO₃⁻, —NR₁₈SO₃₋, or —OPO₃²⁻.

67. The compound of any one claims 44-66, wherein R₆ or R₇ is —OSO₃⁻, —NR₁₈SO₃⁻, or —OPO₃²⁻.

68. The compound of any one of claims 44-67, wherein R₆ or R₇ is —OSO₃⁻.

69. The compound of claim 67, wherein R₇ and R₁₂ are independently —OSO₃⁻.

70. The compound of any one of claims 44-64, wherein R₆, R₇, and R₁₂ are independently H, —OH, —OSO₃⁻, —NR₁₈SO₃⁻, or —OPO₃²⁻, provided that at least one of R₆, R₇, and R₁₂ is —OSO₃⁻, —NR₁₈SO₃⁻, or —OPO₃²⁻.

71. The compound of claim 70, wherein at least one of R₆, R₇ and R₁₂ is —OSO₃⁻, —NR₁₈SO₃⁻, or —OPO₃²⁻.

72. The compound of claim 71, wherein R₆ or R₇ is —OSO₃⁻, —NR₁₈SO₃⁻, or —OPO₃²⁻.

73. The compound of claim 66, wherein R₆ or R₇ is —OSO₃⁻.

74. The compound of claim 65, wherein R₇ and R₁₂ are independently —OSO₃⁻.

75. The compound of any one of claims 44-64, wherein R₇ is substituted or unsubstituted alkyl.

76. The compound of claim 75, wherein R₇ is C(R₁₈)₂SO₃⁻.

77. The compound of any one of claims 44-64, wherein R₇ is —SO₂N(R₁₈)₂.

78. The compound of any one of claims 44-64, wherein R₇ is —OSO₂N(R₁₈)₂.

79. The compound of any one of claims 44-64, wherein R₇ is —NR₁₈SO₃⁻.

80. The compound of any one of claims 75-79, wherein R₁₈ is H.

81. The compound of any one of claims 75-79, wherein R₁₈ is benzyl.

82. The compound of any one of claims 44-64, wherein R₇ is —PO₃Bn₂.

83. The compound of any one of claims 44-82, wherein R₁₉ is TBS.

84. The compound of any one of claims 44-83, wherein R₃ is —OTBS.

85. The compound of any one of claims 44-84, of the formula: 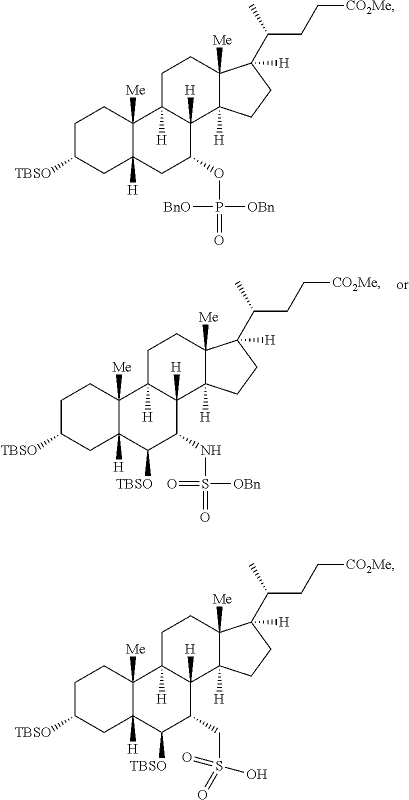
or a pharmaceutically acceptable salt thereof.

86. A pharmaceutically acceptable salt of the compound of any one of claims 44-85.

87. The pharmaceutically acceptable salt of claim 86, wherein the pharmaceutically acceptable salt is an ammonium salt.

88. The pharmaceutically acceptable salt of claim 86, wherein the pharmaceutically acceptable salt is a sodium salt.

89. A pharmaceutical composition comprising a compound of any one of claims 44-85, or a salt of any one of claims 86-88, and an additional pharmaceutical agent.

90. The pharmaceutical composition of claim 89, wherein the composition is formulated for oral administration.

91. The pharmaceutical composition of claim 89 or 90, wherein the pharmaceutical composition is formulated to restrict delivery of the compound to the gastrointestinal tract of the subject.

92. The pharmaceutical composition of any one of claims 89-91, wherein the carrier or excipient restricts delivery of the composition to the gastrointestinal tract.

93. The pharmaceutical composition of any one of claims 89-92, wherein the composition is a solid dosage or liquid dosage form.

94. The pharmaceutical composition of any one of claims 89-93, wherein the compound is an agonist of TGR5.


SEQUENCE LISTING (7 sequences)

SEQ ID NO: 1 - DNA, 1743 bp, Homo sapiens
ctttccgcctagtgagaggcggtccgatttggcccttggggagtgtccgtcgcgttgatctgatggattcacgtacacaacaccacattctatgagattttgcaggcaaaagtccacaagctcgatatatgggacacctgcaccggcattggatttggccccgcaacatcttaaaggaagcaggctgtgagccaaggggaaggcagaggacagaaatgaatgtgtttccaggctttcctggtggtttatggcattctccaaactcctatgcaagggctattcctgaccaagaagatctaaagagaacgtctctgaaatcaagtccggatgaagaattaagagaaaaaaagtgaatatggtttttgctcacagaatggataacagcaagccacatttgattattcctacacttctggtgcccctccaaaaccgcagctgcactgaaacagccacacctctgccaagccaatacctgatggaattaagtgaggagcacagttggatgagcaaccaaacagaccttcactatgtgctgaaacccggggaagtggccacagccagcatcttctttgggattctgtggttgttttctatcttcggcaattccctggtttgtttggtcatccataggagtaggaggactcagtctaccaccaactactttgtggtctccatggcatgtgctgaccttctcatcagcgttgccagcacgcctttcgtcctgctccagttcaccactggaaggtggacgctgggtagtgcaacgtgcaaggttgtgcgatattttcaatatctcactccaggtgtccagatctacgttctcctctccatctgcatagaccggttctacaccatcgtctatcctctgagcttcaaggtgtccagagaaaaagccaagaaaatgattgcggcatcgtggatctttgatgcaggctttgtgacccctgtgctctttttctatggctccaactgggacagtcattgtaactatttcctcccctcctcttgggaaggcactgcctacactgtcatccacttcttggtgggctttgtgattccatctgtcctcataattttattttaccaaaaggtcataaaatatatttggagaataggcacagatggccgaacggtgaggaggacaatgaacattgtccctcggacaaaagtgaaaactatcaagatgttcctcattttaaatctgttgtttttgctctcctggctgccttttcatgtagctcagctatggcacccccatgaacaagactataagaaaagttcccttgttttcacagctatcacatggatatcctttagttcttcagcctctaaacctactctgtattcaatttataatgccaattttcggagagggatgaaagagactttttgcatgtcctctatgaaatgttaccgaagcaatgcctatactatcacaacaagttcaaggatggccaaaaaaaactacgttggcatttcagaaatcccttccatggccaaaactattaccaaagactcgatctatgactcatttgacagagaagccaaggaaaaaaagcttgcttggcccattaactcaaatccaccaaatacttttgtctaagttctcattctttcaattgttatgcaccagagattaaaaagctttaactataaaaacagaagctatttacatatttgttttcactcaactttccaagggaaatgttttattttgtaaaatgcattcatttgtttactgta

SEQ ID NO: 2 - protein, 415 aa, Homo sapiens
MVFAHRMDNSKPHLIIPTLLVPLQNRSCTETATPLPSQYLMELSEEHSWMSNQTDLHYVLKPGEVATASIFFGILWLFSIFGNSLVCLVIHRSRRTQSTTNYFVVSMACADLLISVASTPFVLLQFTTGRWTLGSATCKVVRYFQYLTPGVQIYVLLSICIDRFYTIVYPLSFKVSREKAKKMIAASWIFDAGFVTPVLFFYGSNWDSHCNYFLPSSWEGTAYTVIHFLVGFVIPSVLIILFYQKVIKYIWRIGTDGRTVRRTMNIVPRTKVKTIKMFLILNLLFLLSWLPFHVAQLWHPHEQDYKKSSLVFTAITWISFSSSASKPTLYSIYNANFRRGMKETFCMSSMKCYRSNAYTITTSSRMAKKNYVGISEIPSMAKTITKDSIYDSFDREAKEKKLAWPINSNPPNTFV

SEQ ID NO: 3 - protein, 30 aa, Homo sapiens
HAEGTFTSDVSSYLEGQAAKEFIAWLVKGR

SEQ ID NO: 4 - protein, 427 aa, Homo sapiens
MEAMAASTSLPDPGDFDRNVPRICGVCGDRATGFHFNAMTCEGCKGFFRRSMKRKALFTCPFNGDCRITKDNRRHCQACRLKRCVDIGMMKEFILTDEEVQRKREMILKRKEEEALKDSLRPKLSEEQQRIIAILLDAHHKTYDPTYSDFCQFRPPVRVNDGGGSHPSRPNSRHTPSFSGDSSSSCSDHCITSSDMMDSSSFSNLDLSEEDSDDPSVTLELSQLSMLPHLADLVSYSIQKVIGFAKMIPGFRDLTSEDQIVLLKSSAIEVIMLRSNESFTMDDMSWTCGNQDYKYRVSDVTKAGHSLELIEPLIKFQVGLKKLNLHEEEHVLLMAICIVSPDRPGVQDAALIEAIQDRLSNTLQTYIRCRHPPPGSHLLYAKMIQKLADLRSLNEEHSKQYRCLSFQPECSMKLTPLVLEVFGNEIS

SEQ ID NO: 5 - DNA, 4669 bp, Homo sapiens
ctgcttgtcaaaaggcggcagcggagccgtgtgcgccgggagcgcggaacagcttgtccacccgccggccggaccagaagcctttgggtctgaagtgtctgtgagacctcacagaagagcacccctgggctccacttacctgccccctgctccttcagggatggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaaggagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaagccggacacagcctggagctgattgagcccctcatcaagttccaggtgggactgaagaagctgaacttgcatgaggaggagcatgtcctgctcatggccatctgcatcgtctccccagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctgactaggacagcctgtggcggtgcctgggtggggctgctcctccagggccacgtgccaggcccggggctggcggctactcagcagccctcctcaccccgtctggggttcagcccctcctctgccacctcccctatccacccagcccattctctctcctgtccaacctaacccctttcctgcgggcttttccccggtcccttgagacctcagccatgaggagttgctgtttgtttgacaaagaaacccaagtgggggcagagggcagaggctggaggcagggccttgcccagagatgcctccaccgctgcctaagtggctgctgactgatgttgagggaacagacaggagaaatgcatccattcctcagggacagagacacctgcacctccccccactgcaggccccgcttgtccagcgcctagtggggtctccctctcctgcctactcacgataaataatcggcccacagctcccaccccacccccttcagtgcccaccaacatcccattgccctggttatattctcacgggcagtagctgtggtgaggtgggttttcttcccatcactggagcaccaggcacgaacccacctgctgagagacccaaggaggaaaaacagacaaaaacagcctcacagaagaatatgacagctgtccctgtcaccaagctcacagttcctcgccctgggtctaaggggttggttgaggtggaagccctccttccacggatccatgtagcaggactgaattgtccccagtttgcagaaaagcacctgccgacctcgtcctccccctgccagtgccttacctcctgcccaggagagccagccctccctgtcctcctcggatcaccgagagtagccgagagcctgctcccccaccccctccccaggggagagggtctggagaagcagtgagccgcatcttctccatctggcagggtgggatggaggagaagaattttcagaccccagcggctgagtcatgatctccctgccgcctcaatgtggttgcaaggccgctgttcacccacagggctaagagctagcgctgccgcaccccagagtgtgggaagggagagcggggcagtctcgggtggctagtcagagagagtgtttgggggttccgtgatgtagggtaaggtgccttcttattctcactccaccacccaaaagtcaaaaggtgcctgtgaggcaggggcggagtgatacaacttcaagtgcatgctctctgcagccagcccagcccagctggtgggaagcgtctgtccgtttactccaaggtggggtctttgtgagagtgagctgtaggtgtgcgggaccggtacagaaaggcgttcttcgaggtggatcacagaggcttcttcagatcagtgcttgagtttggggaatgcggccgcattccctgagtcaccaggaatgttaaagtcagtgggaacgtgactgccccaactcctggaagctgtgtccttgcacctgcatccgtagttccctgaaaacccagagaggaatcagacttcacactgcaagagccttggtgtccacctggccccatgtctctcagaattcttcaggtggaaaaacatctgaaagccacgttccttactgcagaatagcatatatatcgcttaatcttaaatttattagatatgagttgttttcagactcagactccatttgtattatagtctaatatacagggtagcaggtaccactgatttggagatatttatggggggagaacttacattgtgaaacttctgtacattaattattattgctgttgttattttacaagggtctagggagagacccttgtttgattttagctgcagaacgtattggtccagcttgctcttcagtgggagaaaacacttgtaagttgctaaacgagtcaatcccctcattcaggaaaactgacagaggagggcgtgactcacccaagcatatataactagctagaagtgggccaggacaggcccggcgcggtggctcacgcctgtaatcccagcagtttgggaggtcgaggtaggtggatcacctgaggtcgggagttcgagaccaacctgaccaacatggagaaaccctgtctctattaaaaatacaaaaaaaaaaaaaaaaaaaatagccgggcatggtggcgcaagcctgtaatcccagctactcaggaggctgaggcagaagaattgaacccaggaggtggaggttgcagtgagctgagatcgtgccgttactctccaacctggacaacaagagcgaaactccgtcttagaagtggaccaggacaggaccagattttggagtcatggtccggtgtccttttcactacaccatgtttgagctcagacccccactctcattccccaggtggctgacccagtccctgggggaagccctggatttcagaaagagcaagtctggatctgggaccctttccttccttccctggcttgtaactccaccaacccatcagaaggagaaggaaggagactcacctctgcctcaatgtgaatcagaccctaccccaccacgatgtggccctggcctgctgggctctccacctcagccttggataatgctgttgcctcatctataacatgcatttgtctttgtaatgtcaccaccttcccagctctccctctggccctgccttcttcggggaactcctggaaatatcagttactcagccctgggccccaccacctaggccactcctccaaaggaagtctaggagctgggaggaaaagaaaagaggggaaaatgagtttttatggggctgaacggggagaaaaggtcatcatcgattctactttagaatgagagtgtgaaatagacatttgtaaatgtaaaacttttaaggtatatcattataactgaaggagaaggtgccccaaaatgcaagattttccacaagattcccagagacaggaaaatcctctggctggctaactggaagcatgtaggagaatccaagcgaggtcaacagagaaggcaggaatgtgtggcagatttagtgaaagctagagatatggcagcgaaaggatgtaaacagtgcctgctgaatgatttccaaagagaaaaaaagtttgccagaagtttgtcaagtcaaccaatgtagaaagctttgcttatggtaataaaaatggctcatacttatatagcacttactttgttgcaagtactgctgtaaataaatgctttatgcaaaccaaaaaaaaaaaaaaaaaa

SEQ ID NO: 6 - protein, 285 aa, Homo sapiens
MSDDFLWFEGIAFPTMGFRSETLRKVRDEFVIRDEDVIILTYPKSGTNWLAEILCLMHSKGDAKWIQSVPIWERSPWVESEIGYTALSETESPRLFSSHLPIQLFPKSFFSSKAKVIYLMRNPRDVLVSGYFFWKNMKFIKKPKSWEEYFEWFCQGTVLYGSWFDHIHGWMPMREEKNFLLLSYEELKQDTGRTIEKICQFLGKTLEPEELNLILKNSSFQSMKENKMSNYSLLSVDYVVDKAQLLRKGVSGDWKNHFTVAQAEDFDKLFQEKMADLPRELFPWE

SEQ ID NO: 7 - DNA, 1904 bp, Homo sapiens
agcctccagcggtggctacagttgaaaccctcacaccacgcaggaagaggtcatcatcatgtcggacgatttcttatggtttgaaggcatagctttccctactatgggtttcagatccgaaaccttaagaaaagtacgtgatgagttcgtgataagggatgaagatgtaataatattgacttaccccaaatcaggaacaaactggttggctgagattctctgcctgatgcactccaagggggatgccaagtggatccaatctgtgcccatctgggagcgatcaccctgggtagagagtgagattgggtatacagcactcagtgaaacggagagtccacgtttattctcctcccacctccccatccagttattccccaagtctttcttcagttccaaggccaaggtgatttatctcatgagaaatcccagagatgttttggtgtctggttattttttctggaaaaacatgaagtttattaagaaaccaaagtcatgggaagaatattttgaatggttttgtcaaggaactgtgctatatgggtcatggtttgaccacattcatggctggatgcccatgagagaggagaaaaacttcctgttactgagttatgaggagctgaaacaggacacaggaagaaccatagagaagatctgtcaattcctgggaaagacgttagaacccgaagaactgaacttaattctcaagaacagctcctttcagagcatgaaagaaaacaagatgtccaattattccctcctgagtgttgattatgtagtggacaaagcacaacttctgagaaaaggtgtatctggggactggaaaaatcacttcacagtggcccaagctgaagactttgataaattgttccaagagaagatggcagatcttcctcgagagctgttcccatgggaataacgtccaaaacactctggatcttatatggagaatgacattgattctcctgtccttgtacatgtacctgactggggtcattgtgtaagacttattattttatcctgaaaccttaaatatcaaacctctgcatctctgatcccttccttgttaaaagttaccagggttggccaggcacggtggttcatgcctgtaatcccagcactatgggaggccgagacgggcggatcacgaggtcaggagactgagaccatcctggctaacacggtgaaaccccatctctactaaaaatacaaaaaccaaaaaaaattagccaggcgcattggctcatgtctgtaatcccagcactttgggaggtcgggggggtgggggaggatcacggggtcaggagatcgagaccatcctggccaacatgatgaaaccctatctctactaaaaatacaaaaattagccgggcatggtggtgcacgcctatagtcccagctactcgggaggctgaggtaggagaatcgtttgaactcaggaggcagaggttgcaatgagccaagatcgcgccactgcactccagcctgggtgacagagcgagaccgtctcaaaaagaaagaagtgactagggttcagagaaccagggttcaaagcccagggatgcaaaggttgcagtgagttgagtcatgggatcccagacttttttaaatgtttgcaatgtttcccgtttacagaatgctacaagaataatgtacgtactacctaaaaggatgtctaaatgtttgttaataaaaataagaaatagctacagtgacagattttagagcaaaaattagtaataaaaataagaaataaaattacaggagcaattaaggttccatttcttttcatcacatgtatctccacaatttattattgctctgatcttaaggagtgtaatttgcaatctaattttatcctcttactctgtgttgttaaaccatatacaataaacggtgtttatgga